(19) United States
(12) Patent Application Publication
(10) Pub. No.: US 2024/0299393 A1
(43) Pub. Date: Sep. 12, 2024

(54) SMALL MOLECULE DISRUPTORS OF PROTEIN INTERACTIONS IN HISTONE DEACETYLASE COMPLEXES

(71) Applicants: Dana-Farber Cancer Institute, Inc., Boston, MA (US); University of Liege, Liege (BE)

(72) Inventors: Julien Olivet, Thy-Le-Chateau (BE); Soon Gang Choi, Newton, MA (US); Jean-Claude Twizere, Gembloux (BE); Marc Vidal, Gloucester, MA (US); Sara Buhrlage, Somerville, MA (US)

(21) Appl. No.: 18/570,787

(22) PCT Filed: Jun. 17, 2022
(86) PCT No.: PCT/US22/34043

§ 371 Date: Dec. 15, 2023

Related U.S. Application Data
(60) Provisional application No. 63/211,936, filed on Jun. 17, 2021.

(51) Int. Cl. A61K 31/519; A61P 25/18; A61P 35/00

(52) U.S. Cl. CPC A61K 31/519; A61P 25/18; A61P 35/00

(57) ABSTRACT

Disclosed are compounds, compositions, and methods useful for disrupting protein interactions in a histone deacetylase (HDAC) complex in a cell of subject or for treating a disease mediated by a histone deacetylase (HDAC) complex in a subject.

### RELATED APPLICATION

This application claims the benefit of priority to U.S. Provisional Patent Application No. 63/211,936, filed Jun. 17, 2021, which is hereby incorporated by reference in its entirety.

### GOVERNMENT SUPPORT

This invention was made with government support under Grant Nos. P50HG004233, R01GM130885, and R01MH111940 awarded by the National Institutes of Health. The government has certain rights in the invention.

### BACKGROUND

Enzymes do not usually function in isolation but rather form complexes that involve many other proteins. For example, regulation of gene expression upon removal of acetyl groups from histone tails by histone deacetylase (HDAC) enzymes often requires the assembly of functional complexes, consisting of: 1) one or several catalytic HDAC component(s); 2) a large scaffold protein around which subunits of the complex gather; and 3) a transcription factor (TF) that recruits the entire complex at different loci, by binding specific DNA motifs. While the catalytic component of such multiprotein complexes is often well characterized, the role of other subunits is usually not well understood. In addition, it is still unclear why several protein partners are required to ensure proper enzymatic activity of HDAC complexes.

It follows that most chemical probes developed and utilized so far to study the normal and disease biology of such complexes target their enzymatic component(s). However, within any class of enzymes such as HDACs, the promiscuity between the catalytic pockets of different paralogs, involved in distinct complexes coexisting in cells, makes specific inhibition by enzymatic perturbants challenging (FIG. 1A). Consequently, HDAC enzymatic inhibitors affect the transcriptional regulation of large numbers of genes in an uncontrolled manner, which complicates the fundamental understanding of specific HDAC-related processes. This might also partially explain why, so far, FDA-approved HDAC inhibitors have only been used to treat peripheral T-cell lymphomas and multiple myelomas, while attempts in treating other diseases such as solid tumor cancers or Alzheimer's disease have often remained unsuccessful. Importantly, even if an inhibitor targeting a single HDAC paralog could be developed, it would still disrupt the many complexes in which that paralog is involved. Thus, higher specificity is not likely to be obtained by targeting enzymatic activity, but rather by affecting other functionalities, such as those involving protein interactions, that are unique to a particular target complex while leaving other complexes unperturbed (FIG. 1B).

Remarkably, today, only a few reports describe attempts at modulating protein interactions in HDAC complexes. For example, a computational screen using the paired amphipathic helix 2 (PAH2) domain of mammalian SIN3A, a scaffold protein of HDAC complexes, identified avermectin derivatives such as ivermectin and selamectin, as inhibitors of the SIN3A/MAD interaction, with implications in triple negative breast cancers.

Computational screens against the PAH1 domain of mammalian SIN3 also identified compounds perturbing the SIN3/REST-NRSF interaction, with inhibition of medulloblastoma cell growth activities. Similar successful attempts at computationally fitting compounds to various protein domains of SIN3 and then measuring phenotypic effects of these molecules in neuronal model systems such as Huntington's disease have also been described. In addition, a recent study reported a C₂₁-steroidal derivative that exhibits therapeutic activity against T-cell lymphomas by binding to SAP18, another subunit of HDAC complexes. There is a need for an unbiased approach to distinguish between two different modes of HDAC complex perturbations: enzymatic inhibition versus modulation of protein interactions.

### SUMMARY

Disclosed herein are compounds that bind to a scaffold protein of HDAC complexes, and inhibit its interaction with transcription factors at specific chromosomal loci. The compounds have similar biological effects as established HDAC enzymatic inhibitors, while perturbing an order of magnitude fewer genes in mammalian cells as well as in a psychosis mouse model.

One aspect of the invention provides compounds, compositions, and methods useful for disrupting protein interactions in a histone deacetylase (HDAC) complex comprising contacting the HDAC complex with an effective amount of a compound having the structure of Formula (I).

Another aspect of the invention relates to methods of disrupting protein interactions in a histone deacetylase (HDAC) complex in a cell of subject comprising administering to the subject an effective amount of a compound having the structure of Formula (I).

Another aspect of the invention relates to methods of treating a disease mediated by a histone deacetylase (HDAC) complex in a subject in need thereof comprising administering to the subject an effective amount of a compound having the structure of Formula (I).

Another aspect of the invention relates to pharmaceutical compositions comprising a compound of Formula (I).

Accordingly, provided herein is a compound having the structure of Formula (I):

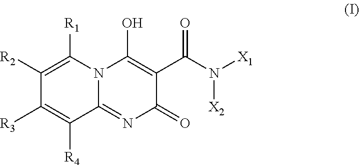

wherein
R₁, R₂, R₃, and R₄ are each independently selected from —H, halo, alkyl, alkenyl, alkynyl, alkoxy, and alkylamino;
X₁ is selected from optionally substituted alkyl, hydoxyalkyl, cycloalkyl, cycloheterocyclyl, aryl, heteroaryl, arylalkyl, and heteroarylalkyl; and
X₂ is selected from —H or alkyl, or a pharmaceutically acceptable salt thereof.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of aspects of the invention will be apparent from the detailed description, and from the claims.

### BRIEF DESCRIPTION OF THE DRAWINGS

FIG. 1A: Enzymatic inhibitors perturb many HDAC complexes.

FIG. 1B: Interaction-specific inhibitors should perturb fewer HDAC complexes.

FIG. 1C: Pipeline to differentiate HDAC complex disruption by enzymatic inhibitors or protein interaction inhibitors. TF=transcription factor; Ctrl=control enzymatic inhibitor; Enz=enzyme; Sub=substrate; Prot=protein.

FIG. 1D: Models of the human SIN3 HDAC (22) and yeast S. cerevisiae Sin3/Rpd3L HDAC (24) complexes where ligandable protein domains and the yeast URS-URA3 reporter gene are represented. Colored subunits indicate orthologous proteins and white subunits proteins lacking full-length orthologs.

FIG. 2A: Yeast S. cerevisiae URS-URA3 reporter assay and Rpd3L HDAC complex as a model system.

FIG. 2B: Expression profile (RT-qPCR) of known Rpd3-regulated genes when perturbations (e.g. deletion of UME6, ume6Δ) are introduced in the Rpd3L HDAC complex (FIG. 29). UBC6 and TAF10 used as housekeeping genes. For each perturbation, the name of the yeast strain, and associated genotype, is indicated (i.e. MaV208/WT). URS=upstream repressing sequence; YPD=yeast extract peptone dextrose medium; RT=room temperature; WT=wild-type. Data represent mean of three replicates. Statistical significance calculated with 2way ANOVA followed by Dunnett's test (compared to WT): *p<0.033, **p<0.0021, ***p<0.0002, ****p<0.0001. Indicated values correspond to the linear fold change over the control.

FIG. 2C: Yeast S. cerevisiae URS-URA3 reporter assay when strains lacking Rpd3L HDAC complex subunits are serially diluted and spotted onto solid synthetic complete (SC), SC lacking uracil (SC-U), or SC+0.2% 5-fluoroorotic acid (5FOA) agar plates.

FIG. 2D: Effect of Rpd3L HDAC complex subunit deletions on known Rpd3-sensitive phenotypes: cycloheximide (CYH) sensitivity at 37° C., and acid phosphatase activity on phosphate-rich (high Pi) medium. Strains were serially diluted, and spotted onto the indicated solid agar plate media. SC-W=SC lacking tryptophan. pDEST-AD-CYH2=plasmid used to restore CYH sensitivity (FIG. 30).

FIG. 2E: Spontaneous Ura⁺ mutants growing when WT URS-URA3 MaV208 yeast cells are plated onto solid SC medium lacking uracil.

FIG. 2F: Characterization of spontaneous Ura⁺ mutants for the URS-URA3 MaV103 yeast strain.

FIG. 2G: Decreasing the number of spontaneous Ura⁺ mutants for the URS-URA3 MaV208 yeast strain by transforming cells with a plasmid containing an extra copy of UME6 (pAR107-UAE6).

FIG. 2H: Improving detection of HDAC inhibitors such as trichostatin A (TSA) or trapoxin A (TRX) when a second copy of UME6 is transformed into URS-URA3 MaV208 yeast cells to obtain the JOY134 strain. The rings of growth on SC-LU (SC lacking leucine and uracil) indicate derepression of URA3 by the compounds.

FIG. 2I: Detection of known HDAC enzymatic inhibitors, in a dose-dependent manner, when they are serially diluted and spotted onto lawns of URS-URA3 JOY134 yeast cells (agar diffusion assay format). The rings of growth on SC-LU (or no growth on 5FOA-containing media) indicate derepression of the URA3 reporter gene by the compounds.

FIG. 2J: Effect of the well-established HDAC enzymatic inhibitors, TSA and TRX, on known Rpd3-sensitive phenotypes: cycloheximide (CYH) sensitivity at 37° C., and acid phosphatase activity on phosphate-rich (high Pi) medium. (

FIG. 2K: Expression profile (RT-qPCR) of known Rpd3-regulated genes when WT URS-URA3 MaV208 yeast cells are treated with TSA or TRX. UBC6 and TAF10 used as housekeeping genes. Data represent mean of four (DMSO) or three (TSA, TRX) replicates. Statistical significance calculated with 2way ANOVA followed by Dunnett's test (compared to DMSO): *p<0.033, **p<0.0021, ***p<0.0002, ****p<0.0001. Indicated values correspond to the linear fold change over the control.

FIG. 2L: Chemical-genetic interactions between known enzymatic inhibitors, TSA and TRX, and deletions of the Pho23 (pho23Δ) or Ume1 (ume1Δ) subunits of the Rpd3L HDAC complex (FIG. 11-13).

FIG. 2M: Expression profiles (RT-qPCR) of five yeast Rpd3-regulated genes compared to URS-URA3 for the deletion mutants presented in (B) (n=3).

FIG. 3A: Schematic of the high-throughput chemical screening to identify HDAC complex perturbants.

FIG. 3B: Retesting the seven verified hits, in a dose-dependent manner, with two different WT URS-URA3 yeast strains: JOY134 and JOY201 (FIG. 29). Compounds were serially diluted and spotted onto lawns of the indicated yeast cells (agar diffusion assay format).

FIG. 3C: Effect of seven verified hits, TSA and TRX, on Rpd3-sensitive acid phosphatase activity. High Pi=phosphate-rich medium FIG. 3D: Expression profile (RT-qPCR) of known Rpd3-regulated genes when WT URS-URA3 MaV208 yeast cells are treated with the indicated compounds, at 250 μM. UBC6 and TAF10 used as housekeeping genes. Data represent mean of four replicates. Statistical significance calculated with 2way ANOVA followed by Dunnett's test (compared to DMSO): *p<0.033, **p<0.0021, ***p<0.0002, ****p<0.0001. Indicated values correspond to the linear fold change over the control.

FIG. 3E: Chemical structure the seven verified hits.

FIG. 3F: Effect of seven verified hits, TSA and TRX, on cycloheximide (CYH)-containing plates at 37° C. Compounds in italic grey were not used for further studies.

FIG. 3G: Chemical-genetic interactions between the seven verified hits, TSA and TRX, and deletions of the Pho23 (pho23Δ) or Ume1 (ume1Δ) subunits of the Rpd3L HDAC complex.

Compounds in italic grey were not used for further studies.

FIG. 3H: Phenotypes of TSA, TRX, and the seven SIN3 HDAC complex perturbants in the URS-URA3 reporter assay. Values represent means of replicates. All source data are provided in FIG. 29.

FIG. 3I: Expression profiles (RT-qPCR) of the six Rpd3-regulated genes from FIG. 2M for the small molecules presented in FIG. 3E (n=4). Values represent means of replicates. Two-way ordinary analysis of variance (ANOVA) with Dunnett's post hoc multiple comparison relative to DMSO: *p<0.033, **p<0.0021, ***p<0.0002, ****p<0.0001. Gene UBC6 used for normalizations. All source data are provided in FIG. 29.

FIG. 3J: (H) Expression profiles (RT-qPCR) of three SIN3 HDAC complex-regulated genes in human neuroblastoma SK-N-BE(2)-C cells for the small molecules presented in previous figure (n≥3). Values represent means of replicates. Two-way ordinary analysis of variance (ANOVA) with Dunnett's post hoc multiple comparison relative to DMSO: *p<0.033, **p<0.0021, ***p<0.0002, ****p<0.0001. Gene RPS11 used for normalizations. All source data are provided in FIG. 29.

FIG. 3K: Inhibition of deacetylase activity by ten classical HDAC enzymatic inhibitors and the seven SIN3 HDAC complex perturbants (n≥3).

FIG. 3L: Inhibition of binary PPIs within the yeast Sin3/Rpd3L HDAC complex by TSA, TRX, and the seven SIN3 HDAC complex perturbants by GPCA (n≥7 for DMSO, n=1 for small molecules).

FIG. 3M: Inhibition of the full-length G. princeps luciferase and yeast Sin3-Ume6 binary PPI by compound E6 in GPCA (n≥3).

FIG. 3N: GPCA versions used to systematically map intra-complex PPIs (top left). Detection of established positive control PPIs (hsPRS-v2) by N₁N₂ GPCA at a selected cutoff of 1.0% recovery rate of negative control protein pairs (hsRRS-v2; FDR=0.01) (n=1). Assay performances from this study are compared to reported data from [Choi et al 2019] at the same cutoff. Mapping binary Sin3/Rpd3L HDAC complex subunit-subunit interactions in a ‘all-by-all’ format (12×12 matrix) using the N₁N₂ GPCA versions (n=1). Protein pairs scoring above the selected 1.0% hsRRS-v2 cutoff are indicated by dark blue cylinders, and light blue cylinders correspond to pairs scoring below that cutoff. The y-axis scale is cut at 150×10³ arbitrary units (a.u.). Bars represent recovery rates (bottom left) or individual data points (right), and error bars standard errors of the proportion (bottom left). Empirical p-value: **p=0.01.

FIG. 3O: KISS assay versions used to retest intra-complex PPIs identified by N₁N₂ GPCA from (left). Retest of intra-complex PPIs from by C₁N₂ KISS versions (n=6) (right). Published NLR value for 1.0% hsRRS-v2 recovery rate from [Choi et al 2019] used as cutoff to score interactions. proportion (B). Empirical p-value: **p=0.01.

FIG. 4A: Measuring inhibition (fold change over DMSO) of HDAC enzymatic activity (HDAC Glo™ I/II assay kit, FIG. 16) by the indicated compounds, at the indicated concentrations, using three different sources of HDACs (live yeast cells, HeLa nuclear extract, semi-purified human HDAC1). Data represent mean of 12 to 24 (DMSO), or three to four (tested compounds) replicates. Statistical significance calculated with 2way ANOVA followed by Dunnett's test (compared to DMSO): *p<0.033, **p<0.0021, ***p<0.0002, ****p<0.0001.

FIG. 4B: Binary protein-protein interaction (PPI) mapping (at a 1.0% hsRRS-v2 scoring threshold) of Rpd3L HDAC (blue) and RNA Pol II (orange) complexes with the G. princeps complementation assay (GPCA) (FIG. 17). Pairs scoring above the 1.0% hsRRS-v2 cutoff are in darker colors and correspond to an empirical p=0.01 (**).

FIG. 4C: Binary PPI map of the Rpd3L HDAC complex. Pairs recovered by GPCA, and validated in the kinase substrate sensor (KISS) assay (1.0% hsRRS-v2 cutoff, empirical p=0.01) are indicated in black and yellow, respectively. Interactions supported by 3D structural data (XRD), or reported in the literature as interacting in binary PPI assays (Lit-BM-17) are indicated in red or purple, respectively.

FIG. 4D: Inhibition (fold change over DMSO) of the Rpd3L HDAC binary PPIs obtained in FIG. 4B by the indicated compounds at 10 μM. For a given protein pair, values framed in red are higher than the average DMSO value minus three standard deviations. Indicated values correspond to the linear fold change over the control.

FIG. 5A: URS-URA3 expression profile after treating WT, ume6Δ, sin3Δ, rpd3Δ or ume6Δ sin3Δ yeast strains with 250 μM TSA or E6. UBC6 and TAF10 used as housekeeping genes. Data represent mean of three replicates. Statistical significance calculated with 2way ANOVA followed by Dunnett's test (compared to DMSO): *p<0.033, **p<0.0021, ***p<0.0002, ****p<0.0001. Indicated values correspond to the linear fold change over the control

FIG. 5B: Pilot ChIP-qPCR assays at URS-URA3, INO1, or UBC6 after treating Sin3-(HA)₃-expressing sin3Δ cells with 400 μM TSA or E6. The arrows on the schematics indicate positions of the primers. Data represent mean of three replicates and standard deviations are shown. Statistical significance calculated with 1 way ANOVA followed by Dunnett's test (compared to DMSO): *p<0.033, **p<0.0021, ***p<0.0002, ****p<0.0001.

FIG. 5C: Benchmarking the Sin3 occupancy map (0.6% DMSO) obtained by ChIP-Seq in this study to published Ume6-bound genes [Rossi et al 2018], and URS-containing genes (from MotifMap, FDR=0.1), when a promoter size cutoff of 600 bp is applied (FIG. 18C).

FIG. 5D: Number of Sin3-occupied genes after treating cells with DMSO (black), TSA (grey), or E6 (red).

FIG. 5E: Overlaps between Sin3-occupied genes from FIG. 5D.

FIG. 5F: Number of Sin3-occupied genes after different treatments that also contain an URS binding site in their promoter, and/or are bound by the Ume6 transcription factor. Comparison with genes randomly picked among the yeast genome.

FIG. 5G: Overlaps between Sin3-occupied genes from FIG. 5F.

FIG. 5H: Disruption of Sin3-containing complexes by hit E6 in yeast S. cerevisiae. ChIP-Seq traces visualized with the Integrative Genomics Viewer (IGV) for a subset of known Rpd3-regulated genes (INO1, IME2, CAR1, SPO13, TRK2), and URS-containing/Ume6-bound genes (INO1, IME2, CAR1, SPO13, SSA1, HSP60, UBI4).

FIG. 5I: Disruption of Sin3-containing complexes by hit E6 in yeast S. cerevisiae. Number of Sin3-occupied genes bound by different transcription factors (TFs) after treating cells with DMSO (black), TSA (grey), or E6 (red). TF-bound genes were identified using the MotifMap online tool (FDR=0.1) [Daily et al 2011].

FIG. 5J: Epistasicity of TSA and E6 with ume6Δ, sin3Δ, rpd3Δ, and sin3Δ/ume6Δ yeast deletion mutants (n=3).

FIG. 5K: Number of loci where Sin3 occupancy is reduced by TSA or E6 in yeast (n=2).

FIG. 5L: Subset of URS-comprising and Ume6-bound loci from FIG. 5K.

FIG. 5M: Number of randomly picked loci not comprising a URS and not bound by Ume6 where Sin3 occupancy is reduced by TSA or E6. Sin3 occupies three of the random loci

FIG. 5N: Examples of ChIP-seq tracks for three URS-comprising and Ume6-bound loci represented by red rectangles. The arrow indicates the sense of the DNA strand and surrounding genes are represented by blue rectangles.

FIG. 6A: Biophysical characterizations of hit E6 and enantiomers. Paired amphipathic helix 2 (PAH2), and Sin3-interacting domain (SID) of the yeast S. cerevisiae Sin3 and Ume6 proteins, respectively.

FIG. 6B: Fluorescence polarization (FP) titration curve showing the interaction of Sin3 PAH2 (N-terminal GST-tagging) with Ume6 SID (N-terminal FITC-tagging) fitted to a K_{D} of 0.86 μM. Data represent mean of three replicates and standard deviations are shown.

FIG. 6C: Crystal structure of the co-complex Sin3 PAH2 (green)-Ume6 SID (yellow) interaction. Right panel: electrostatic surface representation with a gradient from 210 (red) to 10 (blue) kT/e.

FIG. 6D: Amino acids and interactions involved in the Sin3 PAH2-Ume6 SID PPI interface.

FIG. 6E: Enantiomers of hit E6 (racemic mixture): E6S (top) and E6R (bottom).

FIG. 6F: E6, E6S, and E6R compete out FITC-Ume6 SID with the indicated IC₅₀s. An unlabeled Ume6 SID peptide (SID) was used as positive control. Data represent mean of three replicates and standard deviations are shown.

FIG. 6G: Comparison between E6, E6S and E6R (in a dose-dependent manner) in the URS-URA3 reporter assay (agar diffusion assay format).

FIG. 6H: Expression profiles of URS-URA3 and INO1 after treating WT MaV208 yeast cells with different doses of TSA, E6, E6S, and E6R. UBC6 and TAF10 used as housekeeping genes. Data represent mean of three replicates. Statistical significance calculated with 2way ANOVA followed by Dunnett's test (compared to DMSO): *p<0.033, **p<0.0021, ***p<0.0002, ****p<0.0001. Indicated values correspond to the linear fold change over the control.

FIG. 6I: Resonance-specific peak broadening and shifting of E6R in presence (red) compared to the absence (blue) of Sin3 PAH2 in a differential line broadening (DLB) experiment

FIG. 6J: On resonance saturation (red) of Sin3 PAH2 decreases the intensity of the E6R spectrum in a saturation transfer difference (STD) experiment.

FIG. 6K: The average transverse relaxation rate of free E6R (blue) increases in presence of Sin3 PAH2 (red) in a CPMG transverse relaxation (CPMG-R₂) experiment.

FIG. 6L: Regions near 7.4 ppm, 7.25 ppm, and 7.1 ppm as a function of CPMG delay time.

FIG. 6M: In silico docking of E6R into the Ume6-binding region of the yeast S. cerevisiae Sin3 PAH2 domain.

FIG. 6N: Zoom in on the predicted binding of E6R into the deep hydrophobic pocket of Sin3 PAH2.

FIG. 6O: Predicted position of chemical interactions with the indicated amino acids within Sin3 PAH2.

FIG. 6P: ¹⁵N-HSQC NMR spectrum showing assignment of the free Sc Sin3 PAH2 backbone (25 μM). Assigned residues are indicated in red.

FIG. 6Q: Space-filled representation of the Sc Sin3 PAH2 backbone assignment from FIG. 6P. The white circle indicates relative position of the deep hydrophobic pocket formed by helices α1 and α2 and where E6R is predicted to bind.

FIG. 6R: ¹⁵N-SOFAST HMQC NMR spectra of untagged Sc Sin3 PAH2 (25 μM) with or without E6R. The red frame indicates the area on the graph used in FIG. 5N.

FIG. 6S: Scatter plot showing the Sc Sin3 PAH2 (25 μM) residues perturbed (>0.025 ppm split pea green line; >0.05 ppm green line) upon addition of 75 or 250 μM E6R.

FIG. 7A: Total number of differentially expressed genes (upregulated in red, downregulated in blue) for the indicated conditions, when increasing fold-change (FC) over MaV208/WT+DMSO thresholds are applied (adjusted p value <0.05). As indicated by the black frame, a FC threshold >2 was used for the analyses.

FIG. 7B: Overlaps of differentially expressed genes in rpd3Δ and sin3Δ from this study, and those obtained by Kemmeren et al. [Kemmeren et al 2014].

FIG. 7C: Effect of the indicated treatments on the yeast S. cerevisiae transcriptome (upregulated in red, downregulated in blue).

FIG. 7D: Overlaps between differentially expressed genes (p adj <0.05; |log₂(FC)|>1) for the indicated treatments. All, upregulated, and downregulated genes for the different conditions are shown. P values for the intersections are indicated in the tables.

FIG. 7E: Linking phenotypes conferred by TSA, E6, sin3Δ, and rpd3Δ (FIGS. 2C-2D, 3B,3C,3F) to gene expression profiles obtained by RNA-Seq. Effect of E6R on the transcription of genes differentially expressed in WT+TSA, sin3Δ, and rpd3Δ (upregulated in red, downregulated in blue).

FIG. 7F: Number of differentially expressed genes after different treatments, that are occupied by Sin3 (ChIP-Seq; FIG. 5C-5E), or contain an URS binding site in their promoter, and/or are bound by the Ume6 transcription factor.

FIG. 7G: Overlaps between differentially expressed genes from (7F). P values for the intersections are indicated in the tables.

FIG. 7H: Comparing ChIP-Seq (FIG. 5) and RNA-Seq results. Overlaps with differentially expressed genes in sin3Δ.

FIG. 7I: Comparing ChIP-Seq (FIG. 5) and RNA-Seq results. Overlaps with differentially expressed genes in rpd3Δ.

FIG. 7J: Expression profiles (RT-qPCR) of the URS-URA3 reporter gene for different doses of TSA, commercial E6, E6S, and E6R (n≥3).

FIG. 7K: Heat map showing clustering of the different replicates from RNA-seq results in yeast cells (n=3).

FIG. 8A: Effect of E6R on the inhibition of SIN3-containing complexes in mammalian cells. Confirmation of E6R binding to the PAH2 domains of mammalian SIN3A and SIN3B by NMR. Resonance-specific peak broadening and shifting of E6R in presence (red) compared to the absence (blue) of SIN3 PAH2 in a differential line broadening (DLB) experiment. Constructs from human cells were N-terminally tagged with a maltose binding protein (MBP). Hs=H. sapiens; Mm=M. musculus.

FIG. 8B: Effect of E6R on the inhibition of SIN3-containing complexes in mammalian cells. Confirmation of E6R binding to the PAH2 domains of mammalian SIN3A and SIN3B by NMR. On resonance saturation (red) of SIN3 PAH2 decreases the intensity of the E6R spectrum in a saturation transfer difference (STD) experiment. Constructs from human cells were N-terminally tagged with a maltose binding protein (MBP). Hs=H. sapiens; Mm=M. musculus.

FIG. 8C: Effect of E6R on the inhibition of SIN3-containing complexes in mammalian cells. Confirmation of E6R binding to the PAH2 domains of mammalian SIN3A and SIN3B by NMR. The average transverse relaxation rate of free E6R (blue) increases in presence of SIN3 PAH2 (red) in a CPMG transverse relaxation (CPMG-R₂) experiment. Constructs from human cells were N-terminally tagged with a maltose binding protein (MBP). Hs=H. sapiens; Mm=M. musculus.

FIG. 8D: Effect of E6R on the inhibition of SIN3-containing complexes in mammalian cells. Confirmation of E6R binding to the PAH2 domains of mammalian SIN3A and SIN3B by NMR. Regions near 7.4 ppm, 7.25 ppm, and 7.1 ppm as a function of CPMG delay time. Constructs from human cells were N-terminally tagged with a maltose binding protein (MBP). Hs=H. sapiens; Mm=M. musculus.

FIG. 8E: Effect of E6R on the inhibition of SIN3-containing complexes in mammalian cells. Confirmation of E6R binding to the PAH2 domains of mammalian SIN3A and SIN3B by NMR. Resonance-specific peak broadening and shifting of E6R in presence (red) compared to the absence (blue) of SIN3 PAH2 in a differential line broadening (DLB) experiment. Constructs from human cells were N-terminally tagged with a maltose binding protein (MBP). Hs=H. sapiens; Mm=M. musculus.

FIG. 8F: Effect of E6R on the inhibition of SIN3-containing complexes in mammalian cells. Confirmation of E6R binding to the PAH2 domains of mammalian SIN3A and SIN3B by NMR. On resonance saturation (red) of SIN3 PAH2 decreases the intensity of the E6R spectrum in a saturation transfer difference (STD) experiment. Constructs from human cells were N-terminally tagged with a maltose binding protein (MBP). Hs=H. sapiens; Mm=M. musculus.

FIG. 8G: Effect of E6R on the inhibition of SIN3-containing complexes in mammalian cells. Confirmation of E6R binding to the PAH2 domains of mammalian SIN3A and SIN3B by NMR. The average transverse relaxation rate of free E6R (blue) increases in presence of SIN3 PAH2 (red) in a CPMG transverse relaxation (CPMG-R₂) experiment. Constructs from human cells were N-terminally tagged with a maltose binding protein (MBP). Hs=H. sapiens; Mm=M. musculus.

FIG. 8H: Effect of E6R on the inhibition of SIN3-containing complexes in mammalian cells. Confirmation of E6R binding to the PAH2 domains of mammalian SIN3A and SIN3B by NMR. Regions near 7.4 ppm, 7.25 ppm, and 7.1 ppm as a function of CPMG delay time.

FIG. 8I: Effect of E6R on the inhibition of SIN3-containing complexes in mammalian cells. Confirmation of E6R binding to the PAH2 domains of mammalian SIN3A and SIN3B by NMR. Resonance-specific peak broadening and shifting of E6R in presence (red) compared to the absence (blue) of SIN3 PAH2 in a differential line broadening (DLB) experiment.

FIG. 8J: Effect of E6R on the inhibition of SIN3-containing complexes in mammalian cells. Confirmation of E6R binding to the PAH2 domains of mammalian SIN3A and SIN3B by NMR. On resonance saturation (red) of SIN3 PAH2 decreases the intensity of the E6R spectrum in a saturation transfer difference (STD) experiment.

FIG. 8K: Effect of E6R on the inhibition of SIN3-containing complexes in mammalian cells. Confirmation of E6R binding to the PAH2 domains of mammalian SIN3A and SIN3B by NMR. The average transverse relaxation rate of free E6R (blue) increases in presence of SIN3 PAH2 (red) in a CPMG transverse relaxation (CPMG-R₂) experiment.

FIG. 8L: Effect of E6R on the inhibition of SIN3-containing complexes in mammalian cells. Confirmation of E6R binding to the PAH2 domains of mammalian SIN3A and SIN3B by NMR. Regions near 7.4 ppm, 7.25 ppm, and 7.1 ppm as a function of CPMG delay time.

FIG. 8M: In silico docking of E6R into the free mouse M. musculus SIN3B PAH2 domain (PDB: 2F05).

FIG. 8N: Predicted position of chemical interactions with the indicated amino acids within the mouse M. musculus SIN3B PAH2 domain.

FIG. 8O: Effect of E6R on the inhibition of SIN3-containing complexes in mammalian cells. Human SIN3A/B-containing HDAC complexes as model systems in neuroblastoma SK-N-BE(2)-C cells.

FIG. 8P: Expression profile (RT-qPCR) of known HDAC/SIN3-regulated genes (CDKN1A, SOCS3, EGR1) when neuroblastoma SK-N-BE(2)-C cells are treated with the indicated compounds, at 100 μM for 16 h. RPS11 and SNRPD3 used as housekeeping genes. Data represent mean of 12 (DMSO) or three (tested compounds) replicates. Statistical significance calculated with 2way ANOVA followed by Dunnett's test (compared to DMSO): *p<0.033, **p<0.0021, ***p<0.0002, ****p<0.0001. Indicated values correspond to the linear fold change over the control.

FIG. 8Q: Expression profile of CDKNIA, SOCS3, and EGR1 (relative to RPS11) after treating neuroblastoma SK-N-BE(2)-C cells with different doses of TSA or E6R. Data represent mean of 12 (DMSO) or three (tested compounds) replicates. Statistical significance calculated with 2way ANOVA followed by Dunnett's test (compared to DMSO): *p<0.033, **p<0.0021, ***p<0.0002, ****p<0.0001. Indicated values correspond to the linear fold change over the control.

FIG. 8R: Total number of differentially expressed genes (upregulated in red, downregulated in blue) for the indicated conditions, when increasing fold-change (FC) over DMSO thresholds are applied (adjusted p value <0.05). As indicated by the black frame, a FC threshold >2 was used for the analyses.

FIG. 8S: Effect of TSA and E6R on the transcriptome of neuroblastoma SK-N-BE(2)-C cells

FIG. 8T: Effect of TSA and E6R on genes differentially expressed upon TSA or E6R treatments.

FIG. 8U: Overlaps between differentially expressed genes in neuroblastoma SK-N-BE(2)-C cells after treatments with TSA or E6R.

FIG. 8V: Principle of the cell invasion assay.

FIG. 8W: Impact of the indicated compounds on the invasion phenotype of neuroblastoma SK-N-BE(2)-C cells. FBS=fetal bovine serum; SEL=selamectin; IVE=ivermectin. Data represent mean of four replicates (eight for DMSO) and standard errors of mean are shown. Statistical significance calculated by unpaired t-test with Welch's correction (compared to DMSO): *p<0.033, **p<0.0021, ***p<0.0002, ****p<0.0001.

FIG. 9A: Mouse psychosis model.

FIG. 9B: Disruption of SIN3A/B-containing HDAC2 complexes is investigated in the mouse frontal cortex (FC).

FIG. 9C: Mouse liver microsome stability test.

FIG. 9D: Protocol used to generate RT-qPCR and RNA-Seq results from mouse tissues.

FIG. 9E: Number of WT or HDAC2 cKO mice used to generate RNA-Seq results from FC tissues

FIG. 9F: Total number of differentially expressed genes, relative to the WT+vehicle condition, in the mouse FC (upregulated in red, downregulated in blue) for the indicated conditions (FDR=0.3; p<0.05).

FIG. 9G: Overlaps between differentially expressed genes from (9F).

FIG. 9H: The 39 genes whose expression is significantly changed in the FC of WT mice after 50 mg/kg E6R treatment (compared to vehicle). Within each group (n=5 for WT+E6R, or n=5 for WT+vehicle; FIG. 9E), a column represents RNA-Seq results from one mouse. The Homer1 gene is framed in black.

FIG. 9I: Expression of Homer1 (from RNA-Seq results) in the different groups of animals. Data represent box and whiskers with min to max. *p<0.05, **p<0.01 (not corrected for multiple testing).

FIG. 9J: Nine differentially expressed genes in the mouse FC between HDAC2 cKO+vehicle (n=6) and HDAC2 cKO+50 mg/kg E6R (n=6) conditions.

FIG. 9K: Effect of E6R (measured by ChIP-qPCR and compared to vehicle) on the recruitment of the SIN3A-containing HDAC complex at the Homer1 locus in WT. Anti-H3 and anti-IgG were used as positive and negative controls, respectively. Different regions of the Homer1 promoter were checked (right panels). Arrows in the schematics indicate positions of the primers used for ChIP-qPCR. For each group, data represent mean of five (WT) or six (HDAC2 cKO) replicates (FIG. 9E) and standard errors of mean are shown. Statistical significance was calculated by multiple t-tests (compared to vehicle or negative control): *p<0.033, **p<0.0021, ***p<0.0002, ****p<0.0001. TSS=transcription start site; −1.4 kb=1.4 kb upstream of TSS.

FIG. 9L: Effect of E6R (measured by ChIP-qPCR and compared to vehicle) on the recruitment of the SIN3A-containing HDAC complex at the Homer1 locus in (HDAC2 cKO mice. Anti-H3 and anti-IgG were used as positive and negative controls, respectively. Different regions of the Homer1 promoter were checked (right panels). Arrows in the schematics indicate positions of the primers used for ChIP-qPCR. For each group, data represent mean of five (WT) or six (HDAC2 cKO) replicates (FIG. 9E) and standard errors of mean are shown. Statistical significance was calculated by multiple t-tests (compared to vehicle or negative control): *p<0.033, **p<0.0021, ***p<0.0002, ****p<0.0001. TSS=transcription start site; −1.4 kb=1.4 kb upstream of TSS.

FIG. 9M: Principle of the psychedelic-induced head-twitch response (HTR) phenotype in mouse. DOI=2,5-Dimethoxy-4-iodoamphetamine.

FIG. 9N: Protocol followed for measuring HTR phenotype in WT or HDAC2 cKO mice treated or not with 50 mg/kg E6R. The number of mice for each condition is indicated.

FIG. 9O: Impact of the conditional HDAC2 knockout in the mouse FC on DOI-induced HTR, before or after chronic treatment with E6R. Data represent mean of six (WT+E6R) replicates and standard errors of mean are shown. Statistical significance calculated with 2way ANOVA followed by Bonferroni correction: for the 15 min datapoint, *p=0.038; for the pre-versus post-DOI injection: *p=0.012. n.s.=not significant.

FIG. 9P: Impact of the conditional HDAC2 knockout in the mouse FC on DOI-induced HTR, before or after chronic treatment with E6R. Data represent mean of five (WT+vehicle, HDAC2 cKO+vehicle) and standard errors of mean are shown. Statistical significance calculated with 2way ANOVA followed by Bonferroni correction: for the 15 min datapoint, *p=0.038; for the pre-versus post-DOI injection: *p=0.012. n.s.=not significant.

FIG. 9Q: Transcriptomic profiles (RNA-seq) in FC sections of WT or Hdac2 cKO mice treated chronically with E6R or Vehicle, respectively (n≥5). For each condition, a row represents a gene for which the mean between replicates is used.

FIG. 9R: Venn diagrams for the differentially expressed genes presented in FIG. 9D. Common upregulated and downregulated genes are indicated in red and blue, respectively.

FIG. 9S: Individual RNA-seq replicates for the 48 differentially expressed genes in the WT+E6R group compared to Hdac2 cKO+Vehicle and Hdac2 cKO+E6R groups (n≥5). Row-centered log₂ transcripts per million (TPM) values are shown and the blue/red gradients correspond to low/high expression, respectively. Common upregulated and downregulated genes in WT+E6R and Hdac2 cKO+Vehicle groups are highlighted in red and blue, respectively. A black arrow indicates results for the Homer1 gene.

FIG. 9T: Recruitment of SIN3A (ChIP-qPCR) at the Homer1 locus in WT) mice treated chronically with E6R or Vehicle, and compared to α-IgG negative controls (n≥5). Symbols represent independent repeats. Bars represent means of replicates. Bars represent error bars SEM. Multiple t-tests compared to the Vehicle condition:*p<0.033, **p<0.0021, ***p<0.0002, ****p<0.0001. All source data are provided in data S5.

FIG. 9U: Recruitment of SIN3A (ChIP-qPCR) at the Homer1 locus in Hdac2 cKO (mice treated chronically with E6R or Vehicle, and compared to α-IgG negative controls (n≥5). Symbols represent independent repeats. Bars represent means of replicates. Bars represent error bars SEM. Multiple t-tests compared to the Vehicle condition:*p<0.033, **p<0.0021, ***p<0.0002, ****p<0.0001.

FIG. 10A: Benchmarking drug sensitivity in different S. cerevisiae yeast strains. Improved drug sensitivity when two efflux pump-encoding genes, PRD5 and SNQ2, are deleted in the MaV208 yeast strain. Cells were serially diluted, and spotted onto complete media, or cycloheximide (CYH) containing media.

FIG. 10B: Reduction of spontaneous growth of Ura⁺ mutant colonies in the URS-URA3 reporter assay when the WT MaV208 yeast strain is transformed with UME6-expressing plasmid (JOY134 yeast strain).

FIG. 10C: Expression profiles (RT-qPCR) of the URS-URA3 reporter gene in the WT+UAE6 (JOY134) yeast strain relative to the WT (MaV208) strain (n=3). Values represent means of replicates. Two-way ordinary ANOVA with Šidák's post hoc correction relative to WT: **p<0.0001. Gene UBC6 used for normalizations.

FIG. 10D: Phenotypes of TSA and TRX in the URS-URA3 reporter assay.

FIG. 10E: Values represent means of replicates in FIG. 10C.

FIG. 11: Identifying chemical-genetic interactions within the yeast S. cerevisiae Rpd3L HDAC complex. The well-established HDAC enzymatic inhibitors, TSA, TRX, and romidepsin (ROM) were serially diluted and spotted onto the indicated lawns of yeast cells, in an agar diffusion assay format.

FIG. 12A: Validations of the chemical-genetic interactions by rescuing wild-type phenotypes. Complementing pho23Δ with a PH023-expressing plasmid restores a WT phenotype for TSA and TRX.

FIG. 12B: Validations of the chemical-genetic interactions by rescuing wild-type phenotypes. Complementing ume1Δ with a UME1-expressing plasmid restores a WT phenotype for TSA and TRX.

FIG. 13A: Validations of the chemical-genetic interactions by dissecting tetrads.

Dissection of tetrads for crosses between WT and mutant strains: pho23Δ. TSA and TRX were serially diluted and spotted onto the lawns of cells, grown from four different spores. cloNAT=nourseothricin sulfate.

FIG. 13B: Validations of the chemical-genetic interactions by dissecting tetrads. Dissection of tetrads for crosses between WT and mutant strains: ume1Δ. TSA and TRX were serially diluted and spotted onto the lawns of cells, grown from four different spores. cloNAT=nourseothricin sulfate.

FIG. 14: Development and benchmarking of the URS-URA3 JOY200 yeast strain. TSA, ROM, and TRX were serially diluted and spotted onto the different lawns of cells.

FIG. 15: Retesting the 59 representative hits with two different wild-type URS-URA3 yeast strains. Example of retests using the JOY134 and JOY201 yeast strains (FIG. 29) to prioritize compounds.

FIG. 16A: Measuring HDAC enzymatic activity with the HDAC Glo™ I/II assay kit. Chemical reaction for the luminescence-based detection of HDAC enzymatic activity (schematic from Promega).

FIG. 16B: Determining the linear range of signal when life MaV208 yeast cells are used. Data in represent mean of three replicates and standard errors of mean are shown.

FIG. 16C: Determining the linear range of signal when HDAC1 fractions semi-purified from HEK293T cells are used. Data in represent mean of three replicates and standard errors of mean are shown.

FIG. 17A: Benchmarking binary PPI assay versions to map interactions in the Rpd3L HDAC complex. G. princeps complementation assay (GPCA) versions used to map Rpd3L HDAC and RNA Pol II interactions.

FIG. 17B: Detection of individual hsPRS-v2 and hsRRS-v2 protein pairs for the selected assays, at a 1.0% hsRRS-v2 detection cutoff. Comparison between hsPRS-v2 pairs recovered by N1N2 GPCA from this study and from Choi et al 2019 [Choi et al 2019].

FIG. 17C: KISS versions used in this study to validate Rpd3L HDAC interactions identified by GPCA.

FIG. 17D: Validation of the Rpd3L HDAC interactions, tested in both orientations as indicated in (C), by KISS. Pairs scoring above the 1.0% hsRRS-v2 cutoff correspond to an empirical p=0.01 (**). Data represent mean of four replicates. Indicated values correspond to the linear normalized luminescence ratios (NLR).

FIG. 18A: Benchmarking a Sin3-(HA)₃-expressing plasmid for chromatin immunoprecipitation (ChIP) assays in yeast. Schematic of the pull-down of Sin3-(HA)₃ from sin3. yeast cells transformed with the YEplac181-Sin3-(HA)₃ plasmid.

FIG. 18B: Benchmarking complementation of the JOY116/sin3. strain by different Sin3-expressing plasmids. Cells were serially diluted and spotted on the indicated media.

FIG. 18C: Number of Sin3-occupied genes obtained by ChIP-Seq when different promoter size cutoffs are applied.

FIG. 18D: Recruitment of Sin3 (ChIP-qPCR) at the URS-URA3 and INO1 loci following treatment with DMSO control, TSA or E6, and compared to α-IgG negative controls (n≥2). Arrows on the schematics indicate rough positions of the forward and reverse primers. Symbols represent independent repeats, bars means of replicates, and error bars SEM. Overlap between two replicates used. One-way ANOVA with Dunnett's post hoc multiple comparison relative to DMSO control in each pull-down: *p<0.033, *p<0.0021, ***p<0.0002, ****p<0.0001.

FIG. 18E Venn diagram between Sin3-occupied genes from ChIP-seq experiments, URS-comprising genes and Ume6-bound genes. Overlap between two replicates used.

FIG. 18F: Number of URS-comprising yeast loci where Sin3-occupancy is reduced by TSA or E6. Overlap between two replicates used.

FIG. 18G: Number of Ume6-bound yeast loci where Sin3-occupancy is reduced by TSA or E6.

FIG. 19: Isothermal titration calorimetry (ITC) for the Sin3 PAH2-Ume6 SID interaction. Thermodynamic parameters are indicated in the table: enthalpy (AH), entropy (AS), binding stoichiometry (n), and dissociation constant (K_{D}).

FIG. 20A: Crystal structure of the free PAH2 domain of yeast S. cerevisiae Sin3 (tagged with MBP). Tertiary structure representation.

FIG. 20B: Electrostatic surface representation of the apo MBP-tagged Sin3 PAH2 domain with a gradient from 210 (red) to 10 (blue) kT/e.

FIG. 20C: FP titration curve showing the interaction of Sc Sin3 PAH2 domain (GST-tagged) with Ume6 SID (TAMRA-labeled) (n=3). Symbols represent individual data points (A) or means with SEM, and lines fitted curves.

FIG. 20D: FP titration curves showing inhibition of the Sc Sin3 PAH2-Ume6 SID (TAMRA-labeled) interaction by unlabeled Ume6 SID, E6, E6S, or E6R (n=3) with fitted IC₅₀ values of 31.4 μM, 113.0 M, 597.3 μM and 63.7 μM, respectively. Symbols represent individual data points (A) or means with SEM, and lines fitted curves.

FIG. 21: Synthetic plan for E6R and E6S enantiomers.

FIG. 22A: Testing analogs of hit E6 in the URS-URA3 reporter assay.

FIG. 22B: E6 analogs with structures shown. Names shaded in light pink indicate that the corresponding compound was scored positive. Name of the compound provider (according to ICCB-Longwood database) is indicated in parentheses.

FIG. 23: Checking quality of RNA-Seq replicates for yeast S. cerevisiae samples. The three replicates for each individual condition clustered together.

FIG. 24A: Conservation between yeast S. cerevisiae and mammalian SIN3 PAH domains. Sequence alignments for the indicated PAH2 domains. Amino acids circled in blue are conserved between the different species, and are predicted to interact with E6R in the docking models.

FIG. 24B: Comparisons between amino acids interacting with E6R in yeast S. cerevisiae Sin3 PAH2, and mouse M. musculus SIN3B PAH2, according to the docking models.

FIG. 25: Checking quality of RNA-Seq replicates for human neuroblastoma SK-N-BE(2)-C cells samples. The three replicates for each individual condition clustered together.

FIG. 26A: Pilot experiments in mouse with E6R. Expression profile (generated by RT-qPCR and relative to the RPL27 housekeeping gene) of reported HDAC-regulated genes when tissues from the spleen of mice treated with different doses of E6R are tested. For vehicle, n=4, for each tested dose of E6R, n=3. Statistical significance calculated with 2way ANOVA followed by Dunnett's test (compared to vehicle): *p<0.033, **p<0.0021, ***p<0.0002, ****p<0.0001.

FIG. 26B: Pilot experiments in mouse with E6R. Expression profile (generated by RT-qPCR and relative to the RPL27 housekeeping gene) of reported HDAC-regulated genes when tissues from the frontal cortex of mice treated with different doses of E6R are tested. For vehicle, n=4, for each tested dose of E6R, n=3. Statistical significance calculated with 2way ANOVA followed by Dunnett's test (compared to vehicle): *p<0.033, **p<0.0021, ***p<0.0002, ****p<0.0001.

FIG. 26C: Body weight curve for WT or HDAC2 cKO mice treated or not with 50 mg/kg E6R. Data represent mean of five (WT) or six (HDAC2 cKO) replicates and standard errors of mean are shown.

FIG. 26D: Examples of morphologies for the brains and spleens of WT and HDAC2 cKO animals treated or not with 50 mg/kg E6R.

FIG. 27A: Confirmation of E6R binding to yeast S. cerevisiae Sin3 PAH1 and PAH3 domains by NMR. Resonance-specific peak broadening and shifting of E6R in presence (red) compared to the absence (blue) of Sin3 PAH1 in a differential line broadening (DLB) experiment.

FIG. 27B: Confirmation of E6R binding to yeast S. cerevisiae Sin3 PAH1 and PAH3 domains by NMR. On resonance saturation (red) of Sin3 PAH1 decreases the intensity of the E6R spectrum in a saturation transfer difference (STD) experiment.

FIG. 27C: Confirmation of E6R binding to yeast S. cerevisiae Sin3 PAH1 and PAH3 domains by NMR. The average transverse relaxation rate of free E6R (blue) increases in presence of Sin3 PAH1 in a CPMG transverse relaxation (CPMG-R2) experiment.

FIG. 27D: Confirmation of E6R binding to yeast S. cerevisiae Sin3 PAH1 and PAH3 domains by NMR. Regions near 7.4 ppm, 7.25 ppm, and 7.1 ppm as a function of CPMG delay time. PAH1 was N-terminally tagged with MBP. PAH3 was N-terminally tagged with GST. Sc=S. cerevisiae.

FIG. 27E: Confirmation of E6R binding to yeast S. cerevisiae Sin3 PAH1 and PAH3 domains by NMR. Resonance-specific peak broadening and shifting of E6R in presence (red) compared to the absence (blue) of Sin3 PAH3 in a differential line broadening (DLB) experiment.

FIG. 27F: Confirmation of E6R binding to yeast S. cerevisiae Sin3 PAH1 and PAH3 domains by NMR. On resonance saturation (red) of Sin3 PAH3 decreases the intensity of the E6R spectrum in a saturation transfer difference (STD) experiment.

FIG. 27G: Confirmation of E6R binding to yeast S. cerevisiae Sin3 PAH1 and PAH3 domains by NMR. The average transverse relaxation rate of free E6R (blue) increases in presence of Sin3 PAH3 (red) in a CPMG transverse relaxation (CPMG-R₂) experiment.

FIG. 27H: Confirmation of E6R binding to yeast S. cerevisiae Sin3 PAH1 and PAH3 domains by NMR. Regions near 7.4 ppm, 7.25 ppm, and 7.1 ppm as a function of CPMG delay time. PAH1 was N-terminally tagged with MBP. PAH3 was N-terminally tagged with GST. Sc=S. cerevisiae.

FIG. 28A: Confirmation of E6R binding to PAH1 and PAH3 domains of human SIN3A and SIN3B by NMR. Resonance-specific peak broadening and shifting of E6R in presence (red) compared to the absence (blue) of SIN3A/B PAH1 in a differential line broadening (DLB) experiment.

FIG. 28B: Confirmation of E6R binding to PAH1 and PAH3 domains of human SIN3A and SIN3B by NMR. On resonance saturation (red) of SIN3A/B PAH1 decreases the intensity of the E6R spectrum in a saturation transfer difference (STD) experiment.

FIG. 28C: Confirmation of E6R binding to PAH1 and PAH3 domains of human SIN3A and SIN3B by NMR. The average transverse relaxation rate of free E6R (blue) increases in presence of SIN3A/B PAH1 in a CPMG transverse relaxation (CPMG-R₂) experiment.

FIG. 28D: Confirmation of E6R binding to PAH1 and PAH3 domains of human SIN3A and SIN3B by NMR. Regions near 7.4 ppm, 7.25 ppm, and 7.1 ppm as a function of CPMG delay time. All constructs were N-terminally tagged with MBP. Hs=H. sapiens.

FIG. 28E: Confirmation of E6R binding to PAH1 and PAH3 domains of human SIN3A and SIN3B by NMR. Resonance-specific peak broadening and shifting of E6R in presence (red) compared to the absence (blue) of SIN3A/B PAH1 in a differential line broadening (DLB) experiment.

FIG. 28F: Confirmation of E6R binding to PAH1 and PAH3 domains of human SIN3A and SIN3B by NMR. On resonance saturation (red) of SIN3A/B decreases the intensity of the E6R spectrum in a saturation transfer difference (STD) experiment.

FIG. 28G: Confirmation of E6R binding to PAH1 and PAH3 domains of human SIN3A and SIN3B by NMR. The average transverse relaxation rate of free E6R (blue) increases in presence of SIN3A/B PAH1 in a CPMG transverse relaxation (CPMG-R₂) experiment.

FIG. 28H: Confirmation of E6R binding to PAH1 and PAH3 domains of human SIN3A and SIN3B by NMR. Regions near 7.4 ppm, 7.25 ppm, and 7.1 ppm as a function of CPMG delay time. All constructs were N-terminally tagged with MBP. Hs=H. sapiens.

FIG. 28I: Confirmation of E6R binding to PAH1 and PAH3 domains of human SIN3A and SIN3B by NMR. Resonance-specific peak broadening and shifting of E6R in presence (red) compared to the absence (blue) of PAH3 in a differential line broadening (DLB) experiment.

FIG. 28J: Confirmation of E6R binding to PAH1 and PAH3 domains of human SIN3A and SIN3B by NMR. On resonance saturation (red) of PAH3 decreases the intensity of the E6R spectrum in a saturation transfer difference (STD) experiment.

FIG. 28K: Confirmation of E6R binding to PAH1 and PAH3 domains of human SIN3A and SIN3B by NMR. The average transverse relaxation rate of free E6R (blue) increases in presence of PAH3 (red) in a CPMG transverse relaxation (CPMG-R₂) experiment.

FIG. 28L: Confirmation of E6R binding to PAH1 and PAH3 domains of human SIN3A and SIN3B by NMR. Regions near 7.4 ppm, 7.25 ppm, and 7.1 ppm as a function of CPMG delay time. All constructs were N-terminally tagged with MBP. Hs=H. sapiens.

FIG. 28M: Confirmation of E6R binding to PAH1 and PAH3 domains of human SIN3A and SIN3B by NMR. Resonance-specific peak broadening and shifting of E6R in presence (red) compared to the absence (blue) of PAH3 in a differential line broadening (DLB) experiment.

FIG. 28N: Confirmation of E6R binding to PAH1 and PAH3 domains of human SIN3A and SIN3B by NMR. On resonance saturation (red) of PAH3 decreases the intensity of the E6R spectrum in a saturation transfer difference (STD) experiment.

FIG. 28O: Confirmation of E6R binding to PAH1 and PAH3 domains of human SIN3A and SIN3B by NMR. The average transverse relaxation rate of free E6R (blue) increases in presence of PAH3 (red) in a CPMG transverse relaxation (CPMG-R₂) experiment.

FIG. 28P: Confirmation of E6R binding to PAH1 and PAH3 domains of human SIN3A and SIN3B by NMR. Regions near 7.4 ppm, 7.25 ppm, and 7.1 ppm as a function of CPMG delay time. All constructs were N-terminally tagged with MBP. Hs=H. sapiens.

FIG. 29: List of yeast S. cerevisiae yeast strains.

FIG. 30: List of plasmids.

FIG. 31: List of RT-qPCR primers.

FIG. 32: Chemical libraries used. The number of interrogated compounds, primary hits, representative primary hits and verified hits are indicated.

FIG. 33: List of proteins obtained by affinity purification of human HDAC1 followed by mass-spectrometry. HDAC1 (N-terminally FLAG-tagged) was expressed in, and purified from HEK293T cells. The list was compared to known interactors of HDAC1/2 and SIN3A/B subunits from

FIG. 34: List of ChIP-qPCR primers.

FIG. 35: Crystallographic data for apo Sin3 PAH2 and co-complex Sin3 PAH2/Ume6 SID structures. Protein domains from yeast S. cerevisiae.

FIG. 36A: Surface representation of the yeast S. cerevisiae (Sc) Sin3 PAH2 domain in complex with the Ume6 SID peptide at 1.8 Å resolution. The electrostatic surface is presented with a gradient from −3 (red) to +3 (blue) KT/e.

FIG. 36B: Ribbon diagram of the Sc Sin3 PAH2-Ume6 SID structure where alpha helices are indicated.

FIG. 36C: Surface representation of the Sc Sin3 PAH2 domain where residues in contact with the Ume6 SID peptide are indicated in yellow.

FIG. 36D: FP titration curve showing the interaction of Sc Sin3 PAH2 domain (GST-tagged) with Ume6 SID (FITC-labeled) (n=4). Symbols represent means with SEM, and lines fitted curves.

FIG. 36E: FP titration curves showing inhibition of the Sc Sin3 PAH2-Ume6 SID interaction by unlabeled Ume6 SID, E6, E6S, or E6R (n=3). Fitted IC₅₀ and Kᵢ values are indicated in the text. Symbols represent means with SEM, and lines fitted curves.

FIG. 36F: Phenotypes of commercial E6, synthesized E6R+E6S, E6S and E6R in the URS-URA3 reporter assay.

FIG. 36G: Effect of TSA and E6R on Sin3/Rpd3L HDAC complex-regulated genes in yeast (n=3).

FIG. 36H: ¹⁵N-SOFAST HMQC NMR spectra of untagged Sc Sin3 PAH2 (25 μM) with or without E6R. A black arrow indicates the direction of a peak shift with increasing E6R concentrations.

FIG. 36I: Space filling model of the Sc Sin3 PAH2 domain (grey) where carbon atoms of residues experiencing E6R chemical shift perturbations above 0.05 and 0.025 ppm in (H) are colored in green and split pea green, respectively. Oxygen and nitrogen atoms in perturbed residues are shown in red and blue, respectively. The Ume6 SID peptide is visualized as a semi-transparent yellow helix and superposed with the docked E6R structure with a cyan carbon backbone where the red, blue and white colors correspond to oxygen, nitrogen and hydrogen atoms, respectively.

FIG. 36J: Semi-transparent surface representation of the Sc Sin3 PAH2 domain with E6R perturbed residues shown as sticks following the color codes from FIG. 36I.

FIG. 36K: Docking of E6R into the mouse SIN3A PAH2 (PDB: 2L9S) domains where deep blue corresponds to residues conserved across all SIN3 PAH2 homologs and sky blue to semi-conserved residues. Amino acids shown as sticks correspond to NMR perturbed residues by E6R in yeast and their mouse pendants. Symbols represent means with SEM, and lines fitted curves [(D) and (E)]. For each condition, a row represents a gene for which the mean between replicates is used (G). All source data are provided in data S4.

FIG. 36L: Docking of E6R into the mouse SIN3B PAH2 (PDB: 2F05) domains where deep blue corresponds to residues conserved across all SIN3 PAH2 homologs and sky blue to semi-conserved residues. Amino acids shown as sticks correspond to NMR perturbed residues by E6R in yeast and their mouse pendants.

FIG. 37: Crystallographic data for the yeast S. cerevisiae free Sin3 PAH2 domain and co-complex Sin3 PAH2-Ume6 SID structures. A single crystal was used to collect data for each of the structures reported in this study. Values in parentheses are for highest-resolution shell.

FIG. 38A: E6R binds to the conserved human SIN3A and SIN3B PAH2 domains. Profile of amino acid conservation between yeast Sc, mouse M. musculus (Mm) and H. sapiens (Hs) SIN3 PAH2 domains. Residues mapped to the Sc Sin3 PAH2 domain-binding site by NMR are highlighted with stars (chemical shift >0.025 ppm in split pea green, and >0.05 ppm in green). Deepblue corresponds to residues conserved across all SIN3 PAH2 homologs and skyeblue to semi-conserved residues. Residues from Sc Sin3 PAH2 used as reference for numbering.

FIG. 38B: Ligand-detected ¹H-NMR spectra for E6R (300 μM) in presence or absence of the human SIN3A PAH2 domains (10 μM). ¹H-NMR DLB spectra.

FIG. 38C: Ligand-detected ¹H-NMR spectra for E6R (300 μM) in presence or absence of the human SIN3A PAH2 domains (10 μM). ¹H-NMR STD spectra where green curves indicate differences (×5) between intensities.

FIG. 38D: Ligand-detected ¹H-NMR spectra for E6R (300 μM) in presence or absence of the human SIN3A PAH2 domains (10 μM). ¹H-NMR CPMG-R₂ spectra. Regions near 7.4 ppm, 7.25 ppm, and 7.1 ppm from as a function of CPMG delay times (FIG. 38E).

FIG. 38E Ligand-detected ¹H-NMR spectra for E6R (300 μM) in presence or absence of the human SIN3A PAH2 domains (10 μM).

FIG. 38F: Ligand-detected ¹H-NMR spectra for E6R (300 μM) in presence or absence of the human SIN3B PAH2 domains (10 μM). ¹H-NMR DLB spectra.

FIG. 38G: Ligand-detected ¹H-NMR spectra for E6R (300 μM) in presence or absence of the human SIN3B PAH2 domains (10 μM). ¹H-NMR STD spectra where green curves indicate differences (×5) between intensities.

FIG. 38H: Ligand-detected ¹H-NMR spectra for E6R (300 μM) in presence or absence of the human SIN3B PAH2 domains (10 μM). ¹H-NMR CPMG-R₂ spectra.

FIG. 38I: Ligand-detected ¹H-NMR spectra for E6R (300 μM) in presence or absence of the human SIN3B PAH2 domains (10 μM). Regions near 7.4 ppm, 7.25 ppm, and 7.1 ppm from as a function of CPMG delay times (FIG. 38I).

FIG. 39A: E6R reduces neuroblastoma cell invasion and tumor growth in mice while inducing only a few transcriptomic changes. Invasion of human neuroblastoma SK-N-BE(2)-C cells following treatment with the DMSO control, TSA, or E6R (n≥4). Unpaired t-test with Welch's post hoc correction relative to DMSO. Symbols represent independent repeats. Bars represent means of replicates and error bars SEM.

FIG. 39B: Transcriptomic profiles (RNA-seq) of human neuroblastoma SK-N-BE(2)-C cells following treatment with TSA or E6R (n=3). For each condition, a row represents a gene for which the mean between replicates is used.

FIG. 39C: Venn diagrams for the differentially expressed genes presented in FIG. 39B.

FIG. 39D: UMAP plot of scRNA-seq data for human neuroblastoma SK-N-BE(2)-C cells treated with TSA, E6R or DMSO control (46,751 cells total) (n=2). Symbols represent individual cells.

FIG. 39E: Volcano plots showing differentially expressed genes between cells treated with DMSO or E6R, and cells treated with DMSO and TSA from FIG. 39D. Symbols represent differentially expressed genes.

FIG. 39F: Effect of E6R and SAHA on tumor growth in mice xenografted with human neuroblastoma SK-N-BE(2)-C cells (n≥5). paired t-test between treatments: *p<0.033, **p<0.0021, ***p<0.0002, ****p<0.0001. Symbols represent means. Bars represent error bars SEM and lines connecting curves.

FIG. 39G: Expression profiles (RT-qPCR) of three human SIN3 HDAC complex-regulated genes in neuroblastoma SK-N-BE(2)-C cells for different doses of TSA and E6R (n≥3). Two-way ordinary ANOVA with Dunnett's post hoc multiple comparison relative to DMSO (A): *p<0.033, **p<0.0021, ***p<0.0002, ****p<0.0001. Gene UBC6 used for normalizations.

FIG. 39H: Body weight curves of mice xenografted with human neuroblastoma SK-N-BE(2)-C cells, and treated with Vehicle, SAHA or E6R (n=6). Symbols represent means, error bars SEM, and lines connecting curves

FIG. 40: List of proteins obtained after affinity purification followed by mass-spectrometry (AP-MS) analysis of human FLAG-HDAC1 complexes. The list of proteins was compared to known interactors of HDAC1/2 and SIN3A/B subunits from the literature. Yeast S. cerevisiae orthologs were obtained from PANTHER. Proteins identified in at least two out of the three replicates were used to generate the data.

FIG. 41: Plasmids used in disclosed experiments.

FIG. 42: Conditions used for yeast cell selections in disclosed experiments.

### DETAILED DESCRIPTION

### Definitions

For convenience, before further description, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present disclosure to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions described herein may exist in particular geometric or stereoisomeric forms. In addition, polymers may also be optically active. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this disclosure.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid conformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Cycloheteroalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this disclosure, the chemical elements are identified in accordance with the Periodic Table of the Elements, CAS version, Handbook of Chemistry and Physics, 67th Ed., 1986-87, inside cover.

### Exemplary Compounds

One aspect of the invention relates to a compound having the structure of Formula (Ia):

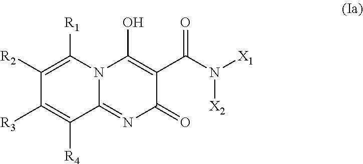

wherein
R₁, R₂, R₃, and R₄ are each independently selected from —H, halo, alkyl, alkenyl, alkynyl, alkoxy, and alkylamino;
X₁ is selected from optionally substituted alkyl, hydoxyalkyl, cycloalkyl, cycloheteroalkyl, aryl, heteroaryl, arylalkyl, and heteroarylalkyl; and
X₂ is selected from —H or alkyl,
or a pharmaceutically acceptable salt thereof,
provided that the compound is not selected from the following compounds:

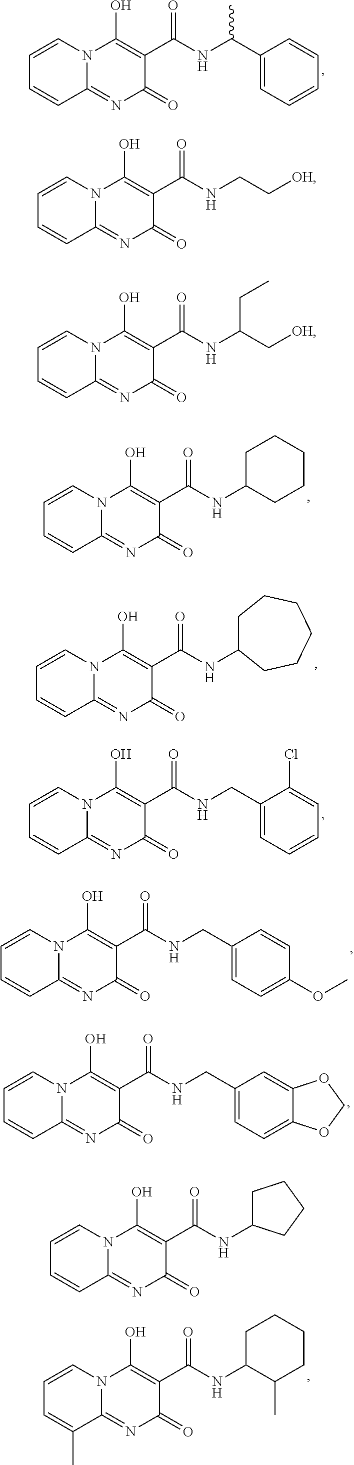

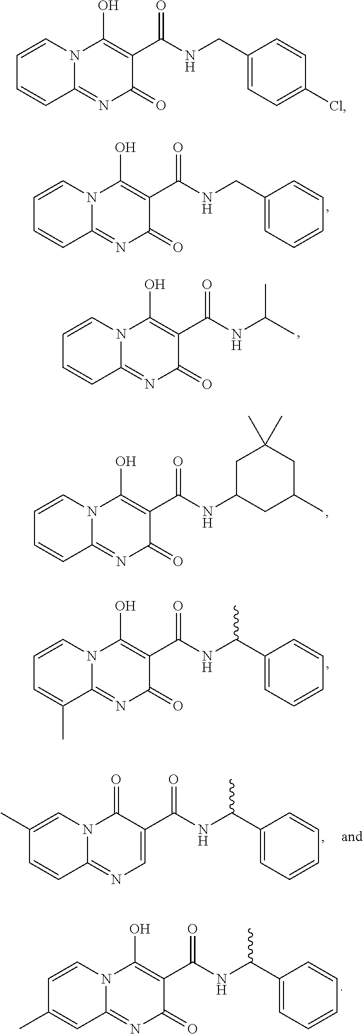

In some embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this disclosure. Such compounds are useful, for example, as analytical tools, as probes in biological assays, or as therapeutic agents. For example, in the case of variable R¹, the (C₁-C₄)alkyl or the —O—(C₁-C₄)alkyl can be suitably deuterated (e.g., —CD₃, —OCD₃).

Any compound described herein can also be radiolabeled for the preparation of a radiopharmaceutical agent.

### Methods of Treatment

One aspect of the invention relates to methods disrupting protein interactions in a histone deacetylase (HDAC) complex in a cell of subject comprising administering to the subject an effective amount of a compound having the structure of Formula (I).

In certain embodiments, the compound disrupts protein interactions in the HDAC complex without substantially inhibiting enzyme activity in the HDAC.

In certain embodiments, the compound inhibits the interactions between a scaffold protein and a transcription factor in the HDAC complex.

In certain embodiments, the HDAC complex comprises a SIN3 scaffold protein.

In certain embodiments, the compound interacts, for example, by binding to a PAH domain of the SIN3 scaffold protein.

In certain embodiments, the HDAC complex comprises a MAD transcription factor.

In certain embodiments, the HDAC complex comprises HDAC1. In other embodiments, the HDAC complex comprises HDAC2.

In certain embodiments, the HDAC complex is a Sin3-HDAC complex.

Another aspect of the invention relates to a method of treating a disease mediated by a histone deacetylase (HDAC) complex in a subject in need thereof comprising administering to the subject an effective amount of a compound having the structure of Formula (I).

In certain embodiments, the compound disrupts protein interactions in the HDAC complex without substantially inhibiting enzyme activity in the HDAC.

In certain embodiments, the compound inhibits the interactions between a scaffold protein and a transcription factor in the HDAC complex.

In certain embodiments, the HDAC complex comprises a SIN3 scaffold protein.

In certain embodiments, the compound interacts with, for example, binds to, a PAH domain of the SIN3 scaffold protein.

In certain embodiments, the HDAC complex comprises a MAD transcription factor.

In certain embodiments, the HDAC complex comprises HDAC1. In other embodiments, the HDAC complex comprises HDAC2.

In certain embodiments, the HDAC complex is a Sin3-HDAC complex.

In certain embodiments, the disease mediated by a histone deacetylase (HDAC) complex is a cancer, a neurodegenerative disease, and a mental disorder. In other embodiments, the disease mediated by a histone deacetylase (HDAC) complex is a neurodegenerative disease. In other embodiments, the disease mediated by a histone deacetylase (HDAC) complex is a mental disorder.

In certain embodiments, the cancer is selected from triple negative breast cancer, T-cell lymphoma, multiple myeloma, a neuroblastoma, and a medulloblastoma.

In certain embodiments, the neurodegenerative disease is Huntington's disease.

In certain embodiments, the mental disorder is selected from psychosis, schizophrenia, and bipolar disorder.

In certain embodiments, the compound has the structure:

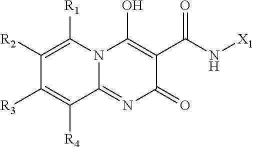

wherein
R₁, R₂, R₃, and R₄ are each independently selected from —H, halo, alkyl, alkenyl, alkynyl, alkoxy, and alkylamino;
X₁ is selected from optionally substituted alkyl, hydoxyalkyl, cycloalkyl, cycloheterocyclyl, aryl, heteroaryl, arylalkyl, and heteroarylalkyl,
or a pharmaceutically acceptable salt thereof.

In certain embodiments, R₁, R₂, R₃, and R₄ are each independently selected from —H and alkyl.

In certain embodiments, R₁, R₂, R₃, and R₄ are each independently selected from —H and —CH₃.

In certain embodiments, one of R₁, R₂, R₃, and R₄ is —CH₃.

In certain embodiments, each of R₁, R₂, R₃, and R₄ is —H.

In certain embodiments, X₁ is optionally substituted alkyl or hydoxyalkyl.

In certain embodiments, X₁ is (C₁-C₆) alkyl.

In certain embodiments, X₁ is a branched (C₁-C₆) alkyl.

In certain embodiments, X₁ is (C₁-C₆) hydoxyalkyl.

In certain embodiments, X₁ is a branched (C₁-C₆) hydoxyalkyl.

In certain embodiments, X₁ is optionally substituted cycloalkyl.

In certain embodiments, X₁ is selected from optionally substituted cyclopentyl, cyclohexyl, and cycloheptyl.

In certain embodiments, the cyclopentyl, cyclohexyl, or cycloheptyl is unsubstituted.

In certain embodiments, the cyclopentyl, cyclohexyl, or cycloheptyl is substituted with at least one alkyl group.

In certain embodiments, the alkyl group is a methyl group.

In certain embodiments, X₁ is selected from

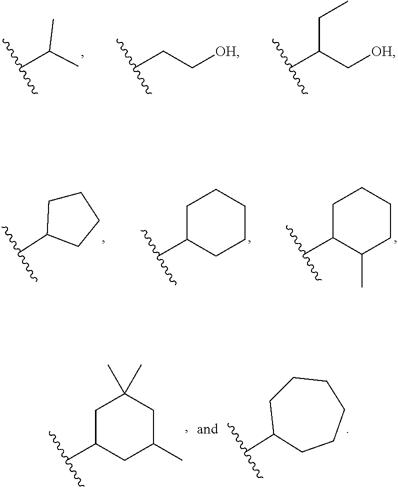

In certain embodiments, X₁ is an optionally substituted arylalkyl.

In certain embodiments, X₁ has the structure:

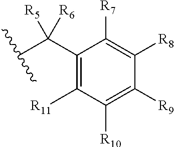

wherein
R₅ and R₆ are each independently selected from —H and alkyl; and
R₇, R₈, R₉, R₁₀, and R₁₁ are each independently selected from —H, halo, alkyl, alkenyl, alkynyl, alkoxy, and alkylamino.

In certain embodiments, R₅ and R₆ are each —H. In other embodiments, one of R₅ and R₆ is H and the other is —CH₃.

In certain embodiments, X₁ has the structure:

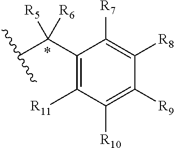

wherein
R₅ and R₆ are each independently selected from —H and alkyl; and
R₇, R₈, R₉, R₁₀, and R₁₁ are each independently selected from —H, halo, alkyl, alkenyl, alkynyl, alkoxy, and alkylamino,
provided that the carbon denoted by * is a chiral center.

In certain embodiments, the * carbon is an R configuration. In other embodiments, the * carbon is an S configuration

In certain embodiments, one of R₅ and R₆ is H and the other is —CH₃.

In certain embodiments, X₁ is selected from

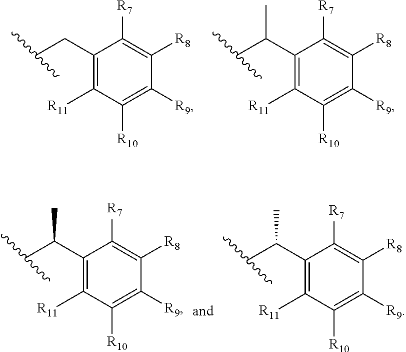

In certain embodiments, R₇, R₈, R₉, R₁₀, and R₁₁ are each independently selected from —H, halo, or alkoxy.

In certain embodiments, R₇, R₈, R₉, R₁₀, and R₁₁ are each independently selected from —H, —Cl, or —OCH₃.

In certain embodiments, R₇, R₈, R₉, R₁₀, and R₁₁ are each —H.

In certain embodiments, X₁ is an optionally substituted heteroarylalkyl.

In certain embodiments, X₁ has the structure:

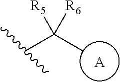

wherein
R₅ and R₆ are each independently selected from —H and alkyl; and
A is an optionally substituted heteroaryl.

In certain embodiments, X₁ has the structure:

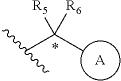

wherein
R₅ and R₆ are each independently selected from —H and alkyl; and
A is an optionally substituted heteroaryl,
provided that the carbon denoted by * is a chiral center.

In certain embodiments, the * carbon is an R configuration. In other embodiments, the * carbon is an S configuration

In certain embodiments, A comprises a nitrogen. In certain embodiments, A comprises an oxygen.

In certain embodiments, A has the structure:

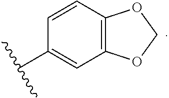 [SMILES: *c1ccc2c(c1)OCO2]

In certain embodiments, the compound of Formula (I) has the structure:

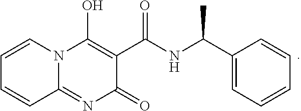 [SMILES: C[C@H](NC(=O)c1c(O)n2ccccc2nc1=O)c1ccccc1]

In certain embodiments, the compound of Formula (I) has the structure:

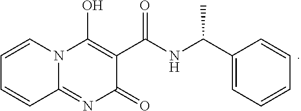 [SMILES: C[C@@H](NC(=O)c1c(O)n2ccccc2nc1=O)c1ccccc1]

In certain embodiments, the compound of Formula (I) is selected from:

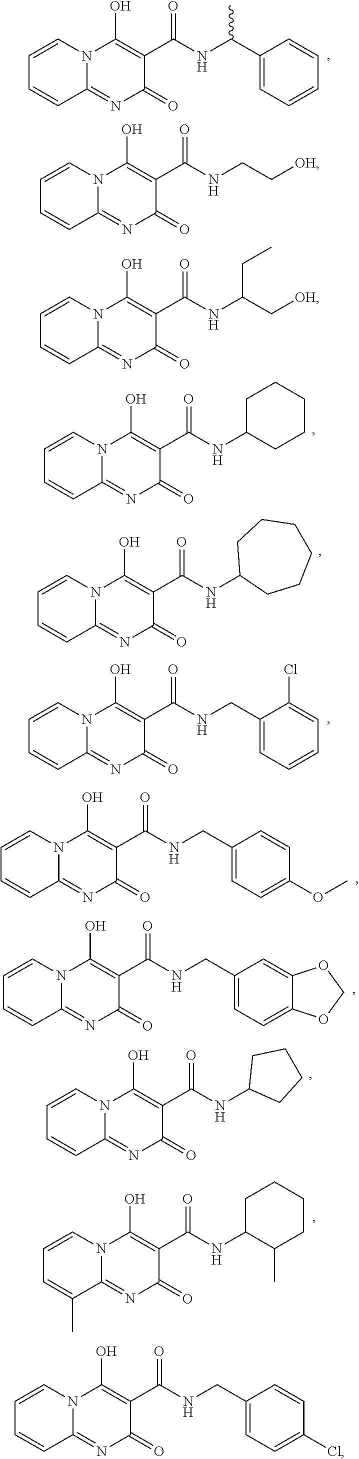

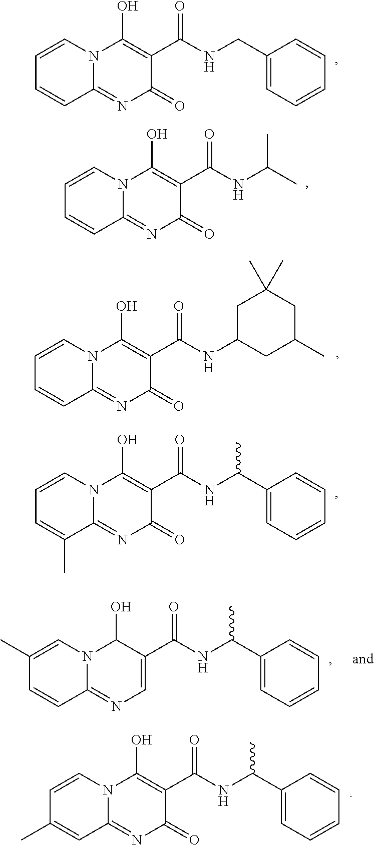

In certain embodiments, the disease mediated by a histone deacetylase (HDAC) complex is partially mediated by a histone deacetylase (HDAC) complex.

In certain embodiments of any one of the disclosed methods, the compound is the compound of Formula (Ia).

In certain embodiments, the compound is not a racemic mixture having the following structure:

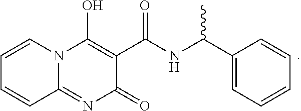 [SMILES: CC(NC(=O)c1c(O)n2ccccc2nc1=O)c1ccccc1]

### Pharmaceutical Compositions, Routes of Administration, and Dosing

In certain embodiments, the invention is directed to a pharmaceutical composition, comprising any of the compounds described herein and a pharmaceutically acceptable carrier. In certain embodiments, the pharmaceutical composition comprises a plurality of compounds described herein and a pharmaceutically acceptable carrier.

Pharmaceutical compositions described herein can be prepared by combining one or more compounds with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion.

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compounds and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions may contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with a compound described herein, and with each other, in a manner such that there is no interaction that would substantially impair the desired pharmaceutical efficiency.

The therapeutic agent(s), including specifically but not limited to a compound described herein, may be provided in particles. Particles as used herein means nanoparticles or microparticles (or in some instances larger particles) which can consist in whole or in part of the compound or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules that contain the compound in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description herein in view of information known to the ordinarily skilled artisan, and may be made without departing from the scope of the disclosure or any embodiment thereof. Having now described the present invention in detail, the same will be more clearly understood by reference to the following examples, which are included herewith for purposes of illustration only and are not intended to be limiting of the invention.

### Materials and Methods

### Media and Conditions for Yeast-Based Assays

The media used in this study were previously described [Yu et al 2008]. For routine cultures of cells not requiring any selection condition, yeast extract-peptone-dextrose (YPD) was used. For other conditions requiring specific selection(s), synthetic complete (SC) media lacking one or several components (SC-L=SC lacking leucine; SC-W=SC lacking tryptophan; SC-U=SC lacking uracil), were used. Cycloheximide (CYH) or 5-fluoroorotic acid (5FOA) were added to the different media at the final concentrations indicated. When the NatMX, KanMX or HphMX cassette was used to delete a specific gene, nourseothricin (clonnat), geneticin (G418), or hygromycin B was added to YPD or SC media to select for mutants. The phosphate-rich (High Pi) medium used for the acid phosphatase plate assay was prepared as previously reported [To et al 1973], with minor modifications. For each liter of high Pi medium: 2 g asparagine, 1.5 g KH₂PO₄, 1.7 g yeast nitrogen base, and 50 mL of 40% glucose were supplemented, with other components that MaV208 yeast cells cannot produce themselves, i.e. 80 mg lysine, 12 mL 40 mM adenine, 8 mL 20 mM uracil, 8 mL 100 mM leucine, 8 mL 40 mM tryptophan, and 8 mL 100 mM histidine. When the temperature is not indicated, yeast cells were grown at 30° C.

### Yeast Transformations

Yeast S. cerevisiae were transformed following a high-efficiency LiAc/SS carrier DNA/PEG protocol [Yu et al 2008]. For each individual transformation reaction, 5 mL of log phase yeast cell culture (OD_{600 nm}˜0.6-1) and ˜0.5 μg of high-purity plasmid, or 5-10 μg PCR-amplified marker cassette, were used. The resulting transformants were plated onto solid SC-L or SC-W, or on YPD supplemented with the proper antibiotics, depending on the plasmid or PCR-amplified marker cassette used. When antibiotics were used in the solid media, transformed cells were inoculated in liquid YPD (30° C.) for 3 h before being plated. Transformants were picked after 3-5 days of incubation at 30° C. for validations or follow up experiments.

Construction of S. cerevisiae Yeast Strains

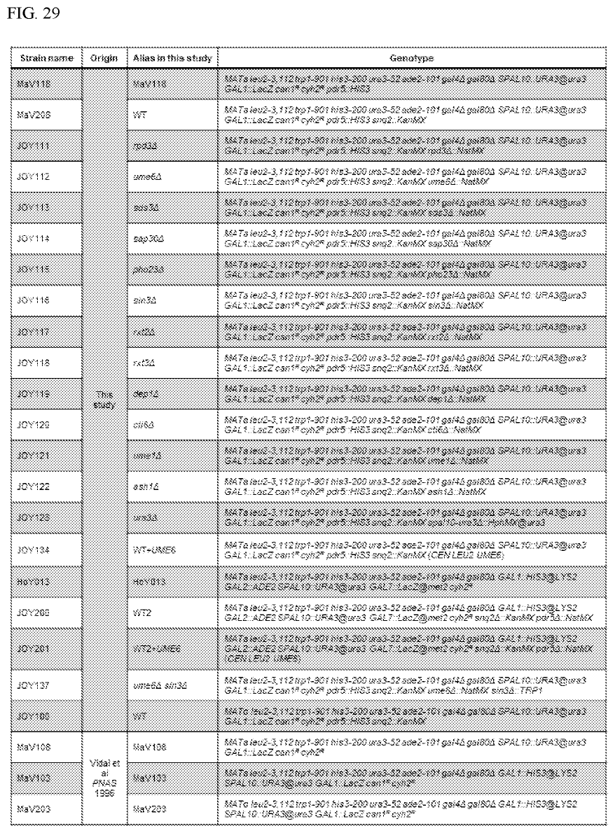

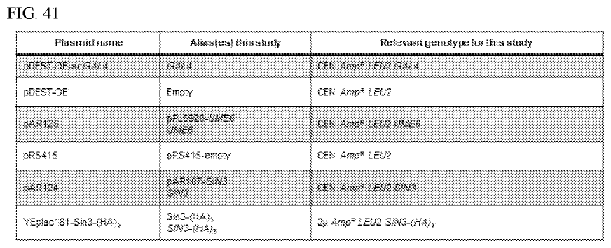

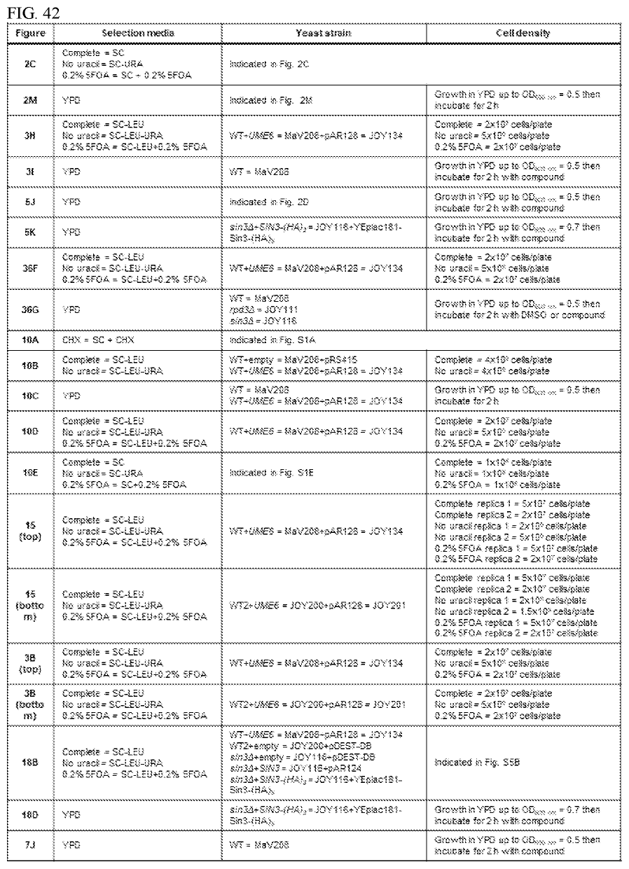

Yeast strains used in this study are presented in FIG. 29, and plasmids in FIG. 41. The different gene deletion strains were generated by double homologous recombination, by transforming yeast cells with gene replacement cassettes as previously described [Baganz et al. 1997, Wendland et al. 2003, Gardner et al 2014]. Briefly, the marker cassettes were PCR-amplified with primers containing 5′-extensions (45-50 bases) directly adjacent to them, and homologous to the promoters or terminators of the targeted genomic loci. The resulting mutants were selected as described above, and single colonies picked and purified before lysing cells and validating the gene deletion/marker cassette by PCR amplification followed by agarose gel electrophoresis using specific primer pairs. MaV208 was generated from MaV108 by disruption of two drug exporter genes using the KanMX gene cassette [Guldener et al 1996], and the HIS3 marker. First, MaV108 was transformed with a DNA fragment that was amplified by PCR reaction using a plasmid pLexA (Clontech) as a template for HIS3 marker. Both ends of the PCR product included a short region homologous to PDRS. Transformed yeast cells were plated on SC plates lacking histidine (SC-HIS) and used to select a new strain named MaV118. This new yeast strain was further transformed with a DNA fragment that was amplified by PCR reaction using the plasmid pUG6 as a template for the KanMX gene cassette [Guldener et al 1996]. Both ends of the PCR product included a short region homologous to KanMX. Transformed cells were plated on YPD plates supplemented with 200 mg/L G418, and a new strain MaV208 was selected from the resistant colonies. For other strains, NatMX, KanMX, and HphMX cassettes were PCR-amplified from plasmids p4339 (gift from Charles Boone, University of Toronto), pRS400, and bRA89 (gift from James E. Haber, Brandeis University), respectively, with primers specific to the TEF promoter and TEF terminator. For the WT+GAL4 control condition, WT MaV208 yeast cells were transformed with a pDEST-DB-scGal4 plasmid [Choi et al 2019] to induce expression of the Gal4 transcription factor and activation of SPAL10::URA3. For JOY134, WT MaV208 yeast cells were transformed with the pAR128 plasmid carrying UME6 (pPL5920-UME6). For JOY128 ((spal10::ura3)Δ::HphMX@ura3), the SPAL10::URA3 region corresponding to the Gal4 binding sites, the URS motif from SPO13, the URA3 reporter gene, and 53 bases of the URA3 terminator, was replaced by the hph marker. The HoY013 strain was generated by inserting the PCR-amplified SPAL10::URA3 reporter system from MaV103 genomic DNA, at the ura3 locus of the Y8800 yeast strain. Y8800 cells were first transformed with a pDEST-DB-scGal4 plasmid before being transformed with the SPAL10::URA3 cassette. Transformants were selected on SC-LEU-URA. After purifying single colonies, the genomic insertion of SPAL10::URA3 was confirmed by PCR with specific primers, and the pDEST-DB-scGal4 was then shuffled out by growing HoY013 cells into complete YPD medium. JOY200 was constructed by deleting PDRS and SNQ2 efflux pumps in HoY013 as explained above.

### Yeast RNA Extraction

After yeast cells reached OD_{600 nm} ˜0.5 in 0.8-1 mL of YPD, they were grown for two more hours at room temperature before being centrifuged to remove supernatant and freezing pellets on dry ice. For compound treatments, yeast cells at OD_{600 nm}˜0.5 in YPD were incubated at room temperature for two hours with DMSO (1-1.6%) or the desired compound (from 25 mM stock in 100% DMSO) in an 2 mL-tube for a final volume of 0.8 mL in YPD, before being centrifuged, washed and frozen as described above. Yeast cells were then harvested for RNA extraction using a RiboPure RNA Purification Kit (Invitrogen, cat #AM1926), which involves mechanical cell wall disruption, phenol extraction of the lysate, and RNA purification using glass fiber, and filter-based RNA purification columns. RNA concentration was measured for individually purified RNA samples using a NanoDrop spectrophotometer. A total of 1 μg RNA from each sample was used for reverse transcription following a published procedure [Choi, S. G. et al 2012].

### Reverse Transcription-Quantitative PCR (RT-qPCR) in Yeast or Mammalian Cells

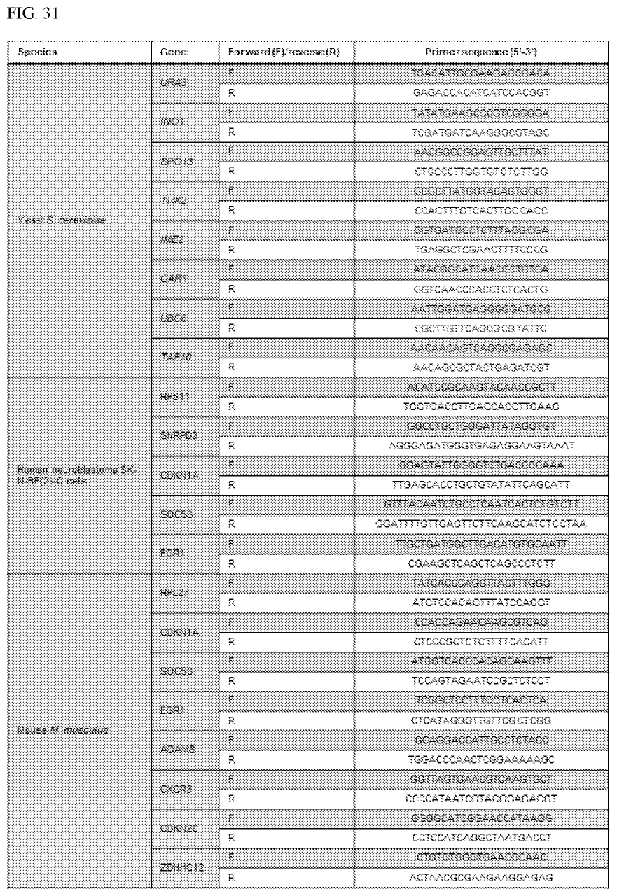

The RT-qPCR protocol used in this study was described elsewhere [Choi et al 2012]. Culture, RNA extraction, and compound treatments for neuroblastoma SK-N-BE(2)-C cells are described below. Approximately 1 μg RNA per sample was used for reverse transcription. Using a thermocycler, RNA was denatured in the presence of 0.2 μg 18 mer oligo-dT by heating the plate at 65° C. for 10 min. The plate was then immediately cooled down on ice to anneal oligo-dT on the poly-A tail of mRNA. A master mix of AffinityScript Multiple Temperature Reverses Transcriptase (Agilent, cat #600107) was prepared following the manufacturer's protocol, and mixed with RNA samples previously annealed with the oligo-dT. The plate was incubated at 42° C. for 2 h to generate cDNAs from mRNA templates. The reverse transcriptase was then denatured by heating the plate at 70° C. for 15 min. The resulting cDNA was diluted (1:20) in sterile, ultra pure water (ddH₂O), and stored at −20° C. until used for qPCR. To measure gene expression, the diluted cDNA was mixed with 5 μL PowerUp SYBR Green master mix (Applied Biosystems, cat #A25743), and the mix of primers (forward+reverse, see FIG. 31; 0.3 μM final concentration) for a total volume of 10 μL. Two technical replicates were generated per cDNA sample in the 384-well PCR plate (Applied Biosystems, cat #4343370). The plate was tightly sealed with optical adhesive film (Applied Biosystems, cat #4311971). Quantitative PCR was conducted with a QuantStudio real-time PCR system from Applied Biosystems, with the following settings: 50° C./2 min, 95° C./10 min, 96° C./15 s/40 cycles, and 60° C./30 s. Dissociation curves were checked for each PCR product, to assess the specificity of the PCR amplicons.

Calculating relative expressions from RT-qPCR assays

Gene expression measured by RT-qPCR was calculated using the following equation:

Expression of gene X normalized to the control gene expression=k*1.96^{(Ct}^{_{Control}}^{-Ct}^{_{Test}}⁾
where k is an arbitrary multiplier (k=1000 in this study), 1.96 is the PCR amplification efficiency constant, Ct is the cycle threshold, Ct_{Control} is the Ct value for the housekeeping gene used as control (UBC6 in yeast [Teste et al 2009]; RPS11 in human cells [Zhan et al 2014], and Ct_{Test} is the Ct value for the tested gene. Replicates corresponding to DMSO treatments or WT yeast cells were averaged and other data points normalized using those DMSO or WT values as references (i.e. expression relative to DMSO or WT). TAF10 in yeast or SNRPD3 in human cells were used as genes not known for being regulated by an HDAC complex (HDAC-unrelated genes).

### Yeast Spot Tests for Mutant Strains

For each strain (FIG. 29), a single colony was picked on a fresh plate, and inoculated in ˜1 mL of the proper liquid selection medium. Cells were grown overnight at 30° C., and under shaking conditions (220 rpm). When cells reached OD_{600 nm} ˜0.6-1, cell densities were evaluated using counting grid microscope slides. They were then centrifuged, supernatants discarded and pellets washed twice with pure water. The resulting pellets were then diluted in different volumes of pure water to obtain similar cell densities throughout the samples. From these starting suspensions, serial dilutions (1:10) were made for every strain, and ˜8-10 μL of culture were spotted for each dilution, on the indicated solid agar media. Plates were then incubated according to the indicated conditions. For plating cells, the washed pellets were resuspended in water at the desired density, and spread out on the agar surface using sterile glass beads.

### Yeast Cycloheximide Sensitivity

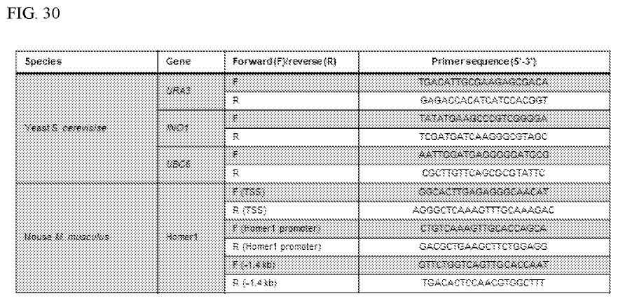

For assessment of cycloheximide (CYH) sensitivity at 37° C., yeast cells with the cyh2^{R} genotype (FIG. 29) were first transformed with an empty pDEST-AD-TRP1-CYH2 plasmid (FIG. 30) before running the different experiments. Selection of the plasmid was maintained by culturing cells in SC-W medium.

### Yeast-Based Acid Phosphatase Plate Assay

The acid phosphatase plate assay was implemented as previously described [To et al 1973, Loewith et al 2001], with minor modifications. Cells were grown on high Pi medium (see above) for 2 days, at 30° C. A staining solution containing 2 mg/mL of 1-naphtyl phosphate (Sigma, N5602) and 4 mg/mL fast blue B salt (Sigma, D9805) was then prepared in 50 mM sodium acetate buffer pH 4.0±0.1, and maintained at 50° C. for ˜30 min before being filtered. Meanwhile, a solution of 10 mg/mL (1% weight/volume) ultra pure low melting point (LMP) agarose (Invitrogen, 15517-022) was prepared in 50 mM sodium acetate buffer pH 4.0±0.1, by microwaving the suspension until complete dissolution of the powder. The volume of the agarose solution was checked and readjusted with 50 mM sodium acetate buffer pH 4.0±0.1. The agarose solution was then mixed with the filtered staining solution by vortexing briefly, and the resulting suspension was kept at 50° C. for ˜15 min. The solid agar plate on which cells grew for 2 days were then gently overlaid with the agarose-containing staining solution, allowing its gelification on top of the cells. A red coloring started to appear ˜2 min after covering cells with the solution.

### Agar Diffusion Assay Format

To identify small molecule perturbants in an agar diffusion assay format, yeast cells were first seeded on the appropriate solid media (dried for at least three days at room temperature), at the indicated cell density per plate. After ˜30 min, compounds (dissolved in 100% DMSO) were then spotted (˜1 to 5 μL) at the indicated concentrations onto the seeded cells, to allow diffusion and formation of a circular concentration gradient around the original spot.

### Yeast Crosses and Tetrad Dissections

Diploids were obtained by mating the two strains of interest on YPD medium overnight, before selecting and growing them on complementation media (SC-LW) for 2-3 days. Fresh diploid colonies were patched onto sterile presporulation GNA plates (for 1 L: 10 g yeast extract, 50 g glucose, 30 g nutrient broth, 20 g agar, and 950 mL water), and grown at 30° C. for one day. Using sterile toothpicks, cells were then transferred from GNA into 2 mL of sporulation medium (0.1 M KOAc: 2.45 g potassium acetate, 250 mL water), and incubated for one day at room temperature (on a roller), and then for 2 days at 30° C. Sporulation was checked under a microscope. Ascus walls were digested as follow: centrifugation of the sporulation mix, gentle removal of the supernatant, gentle resuspension of the pellet into 50 μL of sterile 1 mg/mL zymolase, incubation for 5 min at 30° C., and gentle addition of 700 μL sterile water. From this mix, 30 μL were streaked (single lane) onto a fresh YPD plate from which tetrads were directly dissected, and individual spores isolated using a tetrad dissection microscope.

### Constructing Plasmids Expressing Sin3/Rpd3L HDAC Complex Subunits in Yeast

Plasmid pAR124 was generated by PCR amplifying the SIN3 locus (chrXV: 316440-321724) from S. cerevisiae S288c genomic DNA (Novagen, cat #69240) with tailed primers containing Gateway attB sites and cloning this amplicon into pAR107. Plasmid pAR107 was generated by digesting pRS415 with the restriction enzymes SmaI and SalI (New England Biolabs), PCR amplifying the Gateway cassette from pQZ213 and assembling both products by gap repair in the yeast strain BY4733. Plasmid pAR128 was generated by digesting pPL5920 with MluI-HF and NsiI-HF (New England Biolabs), PCR amplifying the UME6 locus (chrIV: 864920-868020) from S. cerevisiae S288c genomic DNA (Novagen, cat #69240), and ligating both products with the T4 DNA Ligase (New England Biolabs). Plasmid YEplac181-Sin3-(HA)₃ used for ChIP experiments was a gift from David J. Stillman (University of Utah) originally obtained from Kevin Struhl's lab (Harvard Medical School).

High-Throughput Chemical Screening with the Yeast URS-URA3 Reporter Assay

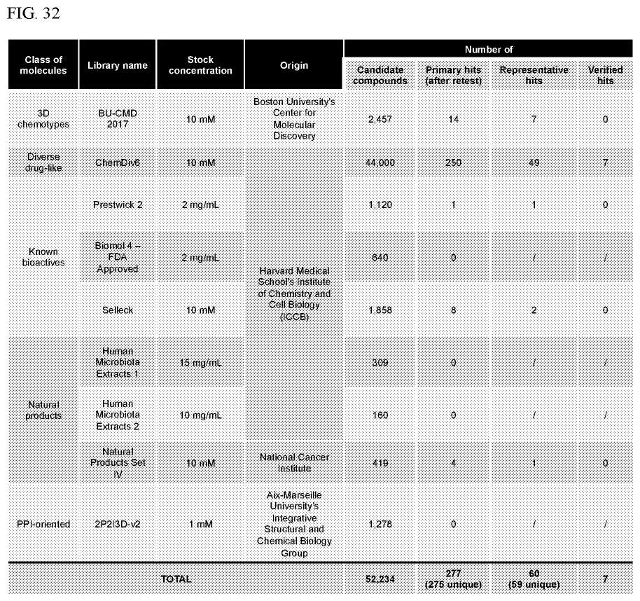

Using the agar diffusion assay format described above, the yeast Rpd3L HDAC complex was interrogated against 52,234 candidate small molecules (FIG. 32), tested in duplicate. JOY134 yeast cells were seeded at a density of ˜5×10⁹ cells per plate on solid SC-LU medium, poured into Nunc OmniTray petri dishes. Using a custom designed Epson Compound Transfer Robot (SGM162) from the Harvard Medical School's Institute of Chemistry and Cell Biology (ICCB) platform, compounds from the selected 384-well format chemical libraries (described in FIG. 32) were pin-spotted (200 nL per spot) using a 100 nL 384-pin head. In addition, 200 nL of DMSO, and trichostatin A (at a concentration of ˜75 mM in DMSO) were manually spotted onto each assay plate. After five to seven days of incubation at 30° C., each duplicated plate was manually scored by comparing the rings of growth from candidate compounds to the DMSO controls. For the retest, selected hits were directly cherry-picked (2 μL per hit) from the library plates used for screening, and the 2 μL of compounds spotted onto JOY134 yeast cells seeded on SC-LU (˜5×10⁹ cells per plate). Primary hits that confer Ura⁺ phenotypes (rings of growth) in this retest experiment (275 distinct in total) were then clustered based on their structural similarities.

Clustering Retested Hits from Screening Based on Structural Similarities

Among the 275 distinct compounds that conferred reproducible Ura+phenotypes in JOY134 yeast cells (i.e. rings of growth on SC-LEU-URA), nine pan assay interference compounds (PAINS) were flagged using a pattern-matching algorithm with a published list of 481 chemical groups as input [Baell et al 2010]. Similarities between structures were then automatically calculated based on functional class fingerprints (FCFP4) with distance to closest using the Tanimoto coefficient. An average cluster size of five compounds was selected, which outputted 56 different groups. The chemical structures within those clusters were then manually inspected and 59 representative small molecules were selected and purchased (dry powders) for retest in a second yeast strain, as described in the main text. Purity of the seven final small molecules selected was confirmed to be >90-95% by liquid chromatography-mass-spectrometry (LC-MS).

### Luminescence-Based HDAC Enzymatic Assays

The HDAC-Glo I/II screening system (Promega, cat #G6430) was adapted to measure the effect of compounds on the enzymatic activity of human and yeast HDAC sources. Purification of recombinant, human HDAC1 was performed from HEK293T cells stably over-expressing an N-terminally FLAG-tagged HDAC1 enzyme, according to Fischle et al [Fischle et al 2002]. FLAG-HDAC1 was eluted from anti-FLAG beads before running the assay, using a FLAG peptide. Nuclear extracts from human HeLa cells were provided in the assay kit. HDAC enzymatic activity was measured following the manufacturer's protocol. Briefly, linear ranges of the assay were determined by measuring luminescence signals, in a serial dilution manner, for semi-purified human HDAC1, or live yeast cells, diluted in the HDAC-Glo I/II buffer. For HeLa nuclear extracts, the manufacturer provided this information in the assay kit. For the different HDAC sources, dilution factors corresponding to signals in the linear ranges were used. To test the effect of compounds (three or four replicates) on HDAC enzymatic activity, equal volumes of HDAC sources (5 μL), and inhibitors (5 μL) were added to the different wells of a white, flat bottom 384-well plate. The plate was then briefly centrifuged with a tabletop centrifuge, and gently shaken for 1 min at 700 rpm. The plate was tightly sealed to prevent evaporation, and incubated at room temperature for 1 h. Following this incubation, 10 μL of luciferase substrate were added to each well. The 384-well plate was then briefly centrifuged, and shaken for 1 min at 700 rpm before being tightly sealed, and incubated for 15 min at room temperature. Luminescence signals were measured using a TriStar luminometer from Berthold Technologies, with 1 s integration time per sample.

### Mass-Spectrometry

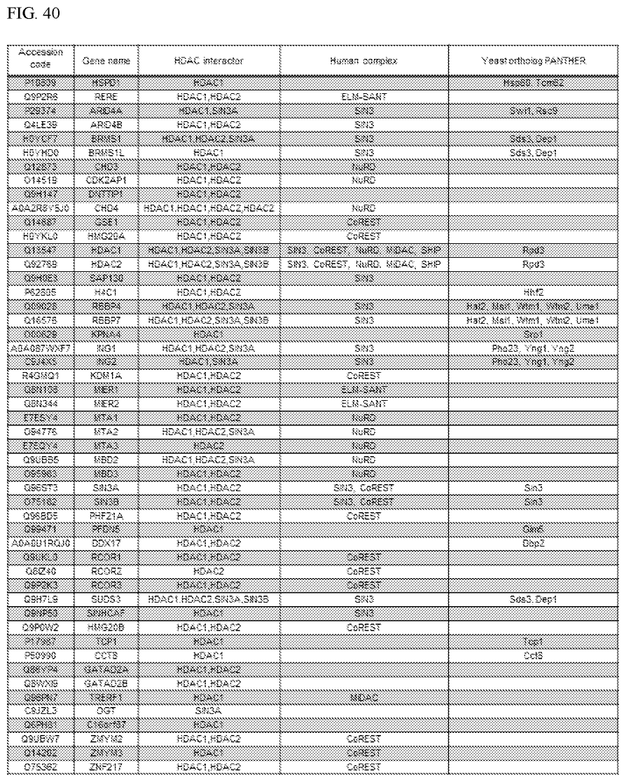

The FLAG-HDAC1 enzymes were immunoprecipitated as described above, using anti-FLAG beads. Beads were treated with DMSO for 1 h, in triplicate, before being washed, and FLAG-HDAC1 enzymes eluted using the FLAG peptide. Proteins from three different replicates were precipitated by methanol/chloroform and digested with trypsin overnight at 37° C., in 50 mM NH₄HCO₃ pH 8.0. Peptides were quantified using a colorimetric peptide assay (Thermo Fisher Scientific). Peptides (1 μg) were dissolved in solvent A (0.1% TFA in 2% ACN), directly loaded onto reversed-phase pre-column (Acclaim PepMap 100, Thermo Scientific) and eluted in backflush mode. Peptide separation was performed using a reversed-phase analytical column (Acclaim PepMap RSLC, 0.075×250 mm, Thermo Scientific) with a linear gradient of 4%-27.5% solvent B (0.1% FA in 98% ACN) for 35 min, 27.5%-50/o solvent B for 10 min, 50%-95% solvent B for 10 min and holding at 95% for the last 5 min at a constant flow rate of 300 nL/min on an EASY-nLC 1000 RSLC system. The peptides were analyzed by an Orbitrap Fusion Lumos Tribrid mass spectrometer (ThermoFisher Scientific). The peptides were subjected to NSI source followed by tandem mass spectrometry (MS/MS) in Fusion Lumos coupled online to the nano-LC. Intact peptides were detected in the Orbitrap at a resolution of 120,000. Peptides were selected for MS/MS using HCD setting at 30; ion fragments were detected in the Ion Trap. A data-dependent procedure that alternated between one MS scan followed by MS/MS scans was applied for 3 s for ions above a threshold ion count of 5.0×10³ in the MS survey scan with 40.0 s dynamic exclusion. The electrospray voltage applied was 2.1 kV. MS1 spectra were obtained with an AGC target of 4×10⁵ ions and a maximum injection time of 50 ms, and MS2 spectra were acquired with an AGC target of 5×10⁴ ions and a maximum injection time of 50 ms. For MS scans, the m/z scan range was 375 to 1800. The resulting MS/MS data was processed using Sequest HT search engine within Proteome Discoverer 2.3 against a human protein database obtained from Uniprot (87,489 entries). Trypsin was specified as cleavage enzyme allowing up to 2 missed cleavages, 4 modifications per peptide and up to 5 charges. Mass error was set to 10 ppm for precursor ions and 0.6 Da for fragment ions. Oxidation on Met, acrylamide on Cys and N-terminal acetylation were considered as variable modifications. False discovery rate (FDR) was assessed using Percolator and thresholds for protein, peptide and modification site were specified at 1%. Abundance ratios were calculated by Label Free Quantification (LFQ) of the precursor intensities within Proteome Discoverer 2.3. Proteins were considered only when they were confidently identified in at least two out of three replicates. Known HDAC1, HDAC2, SIN3A and SIN3B interactors according to mentha [Calderone et al 2013] (downloaded on Aug. 28, 2017) were checked and information on yeast S. cerevisiae orthologs was added according to the PANTHER database (version 16.0; queried on Jan. 28, 2022) to construct FIG. 40. Orthologs marked as “LDO” (least diverged ortholog) or “O” (more diverged ortholog) in PANTHER were considered in FIG. 40.

Cloning ORFs into Gateway-Compatible Expression Plasmids

ORFs corresponding to full-length subunits of the Rpd3L HDAC complex were PCR-amplified from yeast genomic DNA, and cloned into Gateway entry vectors, pDONR223. ORFs for the RNA Pol II complex were collected from the Gateway-compatible Center for Cancer Systems Biology (CCSB) yeast ORFeome collection. Single colonies were picked, and the quality of cloning was checked for every ORF by bi-directional Sanger DNA sequencing. They were then introduced into the different assay-specific expression vectors by LR clonase-mediated Gateway reactions (Life Technologies). LR reaction products were subsequently transformed into DH5a competent bacterial cells and grown for 24 h on ampicillin-containing TFB medium. Plasmid DNA was extracted using a NucleoSpin 96 Plasmid kit from Macherey-Nagel. After PCR-amplification of the cloned ORFs from purified plasmid DNAs with plasmid-specific primers, the size of each DNA amplicon was examined by agarose gel electrophoresis. For hsPRS-v2 and hsRRS-v2 pairs, the GPCA constructions published in [Choi et al 2019] were used. For GPCA, ORFs coding for the Sin3/Rpd3L HDAC complex subunits were cloned into the GPCA-N1 and GPCA-N2 destination plasmids, which allowed expression of the tested proteins, X and Y, as N-terminal fusions of the two luciferase fragments, Luc1 (N₁) and Luc2 (N₂). For KISS, ORFs coding for proteins found in PPIs identified by GPCA were cloned into the KISS-C₁ and N2-KISS destination plasmids to allow expression of the tested proteins, X and Y, as C- and N-terminal fusions, i.e. X-C₁/N₂-Y versus Y-C₁/N₂-X.

Gaussia princeps Complementation Assay (GPCA)

GPCA (N1-N2 version) was implemented as reported in [Choi et al 2019]. HEK293T cells were seeded at 6×10⁴ cells per well in 96-well, flat-bottom, cell culture microplates (Greiner Bio-One, #655083), and cultured in Dulbecco's modified Eagle's medium (DMEM) supplemented with 10% fetal calf serum at 37° C. and 5% CO₂. Twenty-four hours later, cells were transfected with 100 ng of each GPCA plasmid (GPCA-N1 and GPCA-N2) using linear polyethylenimine (PEI) to co-express the protein pairs fused with complementary luciferase fragments, Luc1 and Luc2. The DNA/PEI ratio used for transfection was 1:3 (mass:mass). GPCA vectors carry the human cytomegalovirus (CMV) promoter and are maintained as high copy number with the human virus SV40 replication origin in mammalian cells. Twenty-four hours after DNA transfection, the cell culture medium was removed, cells were gently washed with 150 μL pre-warmed 1×PBS, 40 μL of lysis buffer was then added per well, and cell lysis was performed under vigorous shaking of the plate for 20 min at 900 rpm. Luminescence was measured by auto-injecting 50 μL Renilla luciferase substrate (Renilla Luciferase Assay system, catalog No. E2820, Promega) per well and integrating light output for 4 s using a TriStar luminometer (Berthold).

The stock solution of PEI HCl (PEI MAX 40000; Polysciences Inc; cat #24765) was prepared according to the manufacturer's instructions. Briefly, 200 mg of PEI powder added to 170 mL of water, stirred until complete dissolution, and pH was adjusted to 7 with 1 M NaOH. Water was added to obtain a final concentration of 1 mg/mL, and the stock solution was filtered through a 0.22 μm membrane.

### Kinase Substrate Sensor (KISS) Assay

KISS (C1-N2 version) was implemented as reported in [Choi et al 2019, Lievens et al 2014]. HEK293T cells were cultured as described above for GPCA. Cells were transfected with bait, prey and reporter plasmids (KISS-C1 and N2-KISS) corresponding to empty controls (unfused gp130 tag or unfused TYK2 C-terminal fragment tag) or Rpd3L HDAC interacting pairs (both orientations were tested) initially identified by GPCA, applying a standard calcium phosphate transfection method. Luciferase activity was measured 48 h after transfection using the Luciferase Assay System kit (Promega) on a Enspire luminometer (Perkin-Elmer). The average of four culture wells was used.

Scoring Interactions from GPCA and KISS

For GPCA, a benchmarking experiment using the 60 hsPRS-v2 and 78 hsRRS-v2 pairs published in [Choi et al 2019], was run in parallel to the all-by-all screening of Rpd3L HDAC, and RNA Pol II interactions to set up the random detection threshold of 1.0%. This 1.0% hsRRS-v2 detection cutoff was determined using R quantile function after log 2 transformations of the raw luminescence signals of hsRRS-v2 pairs, which resulted in a theoretical value of 38240.50 units. All protein pairs with luminescence signals above that threshold were scored positive. A p value of 4.01×10⁻¹⁰ was calculated for the set of positive hsPRS-v2 pairs over the hsRRS-v2 random controls using R functions Wilcox.test. The mapping of binary PPIs of the Rpd3L HDAC and RNA Pol II complexes was conducted by pairwise testing all 24 subunits against each other's. Positions of the tested pairs were randomized in the culture plates. Luminescence was measured for all 576 protein pairs (i.e. 24×24 search space), along with the 138 protein pairs from hsPRS-v2/hsRRS-v2. In this all-by-all screen, every protein pair scoring above the 1.0% hsRRS-v2 detection cutoff was associated to an empirical p value=0.01.

For KISS, an NLR cutoff corresponding to exactly 1.0% hsRRS-v2 pair scored positive was applied to the data published in [Choi et al 2019]. This theoretical cutoff of 3.81 was used as reference to validate Rpd3L HDAC interactions from GPCA. A p value of 4.51×10⁻⁷ was calculated, using the recalibrated data, for the set of positive hsPRS-v2 pairs over the hsRRS-v2 random controls using R functions Wilcox.test. The luciferase ratio obtained for each Rpd3L HDAC bait-prey protein pair versus that obtained for the combination of the same bait with a negative control prey (unfused gp130), and versus that obtained for the combination of the same prey with a negative control bait (unfused TYK2 C-terminal fragment) were evaluated against the published, reference NLR corresponding to no hsRRS-v2 pair detection. NLR were calculated as follows, for each protein pair, X-Y:

$${NLR}_{( X - Y )} = \frac{{Luminescence}_{( X - Y )}}{{Luminescence}_{( X - empty ) or ( empty - Y )}} \qquad ( 1 )$$

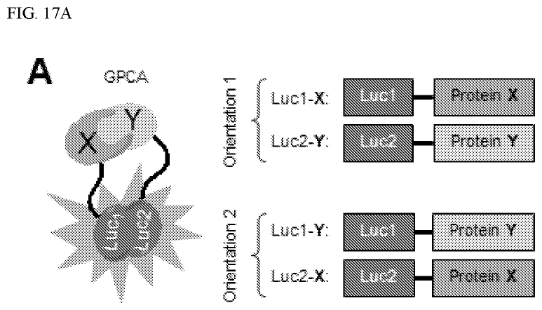

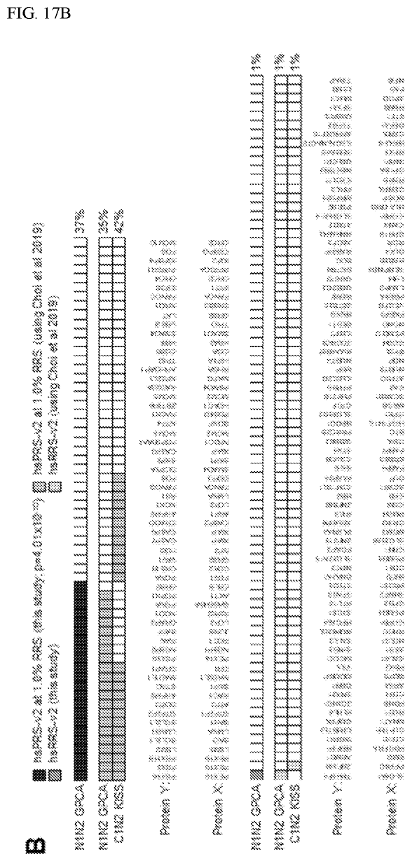

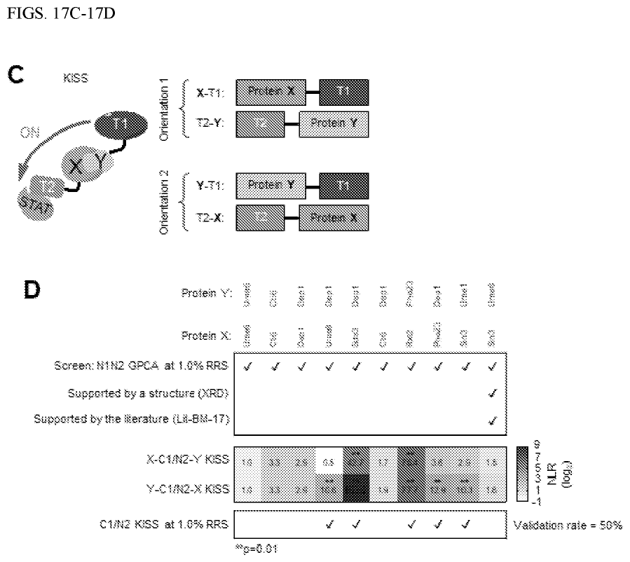

An interaction pair was scored positive when both NLR, using X-empty or empty-Y, exceeded the reference cutoff for either of the two configurations tested, corresponding to empirical p values=0.01. Between the two NLR values of each positive pair and tested orientation, the smallest was used to construct FIG. 17D.
Processing Published hsPRS-v2 and hsRRS-v2 Data

In order to compare published data to results from this study in the same conditions, raw luminescence values for N1-N2 GPCA were first extracted from [Choi et al 2019]. The hsPRS-v2 recovery rate for these experiments was then recalculated using a theoretical hsRRS-v2 detection threshold of exactly 1.0%. As described above, this 1.0% hsRRS-v2 detection cutoff was determined using R quantile function after log 2 transformations of the published raw luminescence signals of hsRRS-v2 pairs, which resulted in a theoretical value of 62745.18 units. Every hsPRS-v2 pair scoring above this signal was considered positive. Published results were then compared to those obtained here, for each individual protein pair, to evaluate the reproducibility of GPCA performances between different studies. KISS data were treated as described above.

Identifying Rpd3L HDAC and RNA Pol II Interactions Supported by the Literature and/or 3D Structures

Yeast S. cerevisiae gene names corresponding to the 12 reported subunits of the Rpd3L HDAC (i.e. UME6, SIN3, RPD3, SDS3, DEP1, SAP30, RXT2, PH023, UME1, ASH1, CTI6, and RXT3), and the 12 protein subunits of the RNA Pol II (i.e. RPO21 alias RPB1, RPB2, RPB3, RPB4, RPBS, RPO26 alias RPB6, RPB7, RPB8, RPB9, RPB10, RPB11, RPC10 alias RPB12) complexes were used to search for published, high-quality binary interactions in the Literature-Binary Multiple-2017, or Lit-BM-17, database, here: http://yeri.baderiab.org/. Structurally supported, direct contacts between subunits of the RNA Pol II complex (23 in total) were extracted from the published structure (PDB: 1WCM) [Armache et al 2005], while the co-complex, crystal structure of the Sin3 PAH2/Ume6 SID domain-domain interaction was solved in this study (see below).

### Testing Binary PPI Inhibition by GPCA

The ability of compounds to inhibit binary PPIs in the Sin3/Rpd3L HDAC complex was tested by GPCA in a high-throughput screening format. Interactions identified in the mapping at 1.0% hsRRS-v2 detection cutoff were reconstituted by expressing protein pairs in the exact same orientations. Briefly, the ten binary PPIs of the Sin3/Rpd3L HDAC complex were tested against DMSO controls (n≥7) (1% of the final volume), TSA, TRX, and the seven confirmed small molecules. HEK293T cells were transfected with 100 ng of each GPCA plasmid expressing either protein X, or protein Y, as described above. 24 h after DNA transfection, cells were treated with 10 μM of compounds, or equivalent DMSO volumes, and incubated for 1 h at 37° C. The luminescence signal for each PPI was measured by averaging the different DMSO replicates, and calculating the standard deviation of DMSO values for each interaction. For each PPI, an individual data point for every compound tested was obtained and this value was compared to the averaged DMSO value. For each interaction, a particular compound was scored as a PPI inhibitor when it decreased its luminescence signal by at least three standard deviations compared to the corresponding averaged DMSO value. The independent retest of compound E6 against the N₁-Sin3/N2-Ume6 interaction and the full-length Gaussia princeps luciferase was done in the linear range of signal. HEK293T cells were seeded at a density of 3×10⁵ cells/mL/well in six-well plates (i.e. 2 mL per well) and cultured in DMEM medium supplemented with 10% FBS at 37° C./5% CO₂. 24 h later, cells were transfected with 2 μg of each subunit-expressing plasmid (i.e. N₁-Sin3, N₂-Ume6), or 1 μg of the full-length Gaussia princeps luciferase-expressing plasmid using linear PEI. The mass/mass DNA/PEI ratio used for transfection was 1:3. 24 h post-transfection, cells were trypsinized and resuspended into the culture medium at a density of 3×10⁵ cells/mL before being seeded at a density of 3.5×10⁴ cells/well (i.e. 100 μL per well) in 96-well, flat-bottom microplates (Greiner Bio-One, cat #655083). After 6 h, cells were treated with the indicated concentrations of the compound (stock concentration of 25 mM in 100% DMSO), for a total volume of 1% DMSO per well (three replicates for each concentration and 11 replicates for DMSO alone). Plates were then incubated for 16 h at 37° C. The medium was removed and the lysis was implemented as described above. 50 μL/well of the Renilla luciferase substrate (Renilla Luciferase Assay system, cat #E2820, Promega) were then added and luminescence was read approximately two minutes after injection using a Centro XS³ LB960 plate reader (3 s integration). For each plate, signal was measured twice and the first measurement was used for calculations. Results were normalized by averaging raw luminescence values from DMSO wells and dividing each data point by this averaged value.

### Chromatin Immunoprecipitation Followed by Quantitative PCR (ChIP-qPCR) or Next-Generation Sequencing (ChIP-Seq) in Yeast

ChIP procedures in yeast were adapted from previously established protocols, with minor modifications [Sertil et al 2007, Jezek et al 2017, Barski et al 2007]. Briefly, yeast S. cerevisiae JOY116 (sin3Δ) cells transformed with the YEplac181-Sin3-(HA)₃ plasmid were cultured in liquid SC-L, until OD_{600 nm} reached ˜0.7. Then, DMSO (0.6%), 400 μM E6, or 400 μM TSA was added to 2×10⁹ yeast cells, and the resulting suspensions were incubated for 2 h at 30° C. (200 rpm). After 2 h of incubation, cells were treated with 1% formaldehyde for 20 min at 4° C. for cross-linking. Then, 125 mM glycine was applied for 5 min at room temperature to quench cross-linking. Yeast cells were centrifuged (10 min at 1,000 g), and the resulting pellets were washed twice with 50 mL ice-cold phosphate-buffered saline (PBS). Yeast pellets were resuspended in 500 μL of ChIP lysis buffer (50 mM HEPES-KOH pH 7.5, 140 mM NaCl, 1 mM EDTA, 1% Triton X-100, 0.1% sodium deoxycholate, 0.1% SDS, and yeast protease inhibitors). After that, 500 μL of the yeast suspensions in lysis buffer were mixed with 500 μL of zirconia beads in a screw-top microcentrifuge tube. For cell lysis, the tubes were vortexed at maximum speed for 15 min at 4° C. Cell lysates were collected by punching a hole on the bottom of the screw-cap tube, with a sterile needle, and centrifuging the lysates into new Eppendorf tubes. Then, 300 μL of lysates were added into 1.5 mL Bioruptor Pico microtubes with caps (Diagenode, cat #C30010016), and subsequent sonication was conducted with a Bioruptor Pico sonication device, in the following conditions: 30 s/3 cycles on, and 30 s/3 cycles off, at 4° C. The sonicated lysates were cleared by 30 min centrifugation at maximum speed at 4° C. The resulting cleared lysates, with sheared chromatin, were saved, snap freezed, and stored at −80° C. until use. Next, 10 μL cleared lysates were reverse-cross linked overnight, at 65° C., in the presence of proteinase K. Phenol-chloroform extractions and EtOH precipitations were then applied to purify the sheared DNA. DNA agarose gel running and DNA concentration measurements with a NanoDrop spectrophotometer were finally conducted to assess the size and yield of the sheared DNA, respectively.

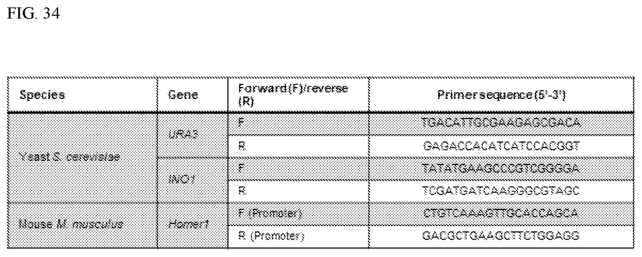

For the pull-downs, 25 μg of sheared chromatin from each sample were diluted (1:10) in ice-cold dilution buffer (1% Triton X-100, 2 mM EDTA, 20 mM Tris-HCl pH 8, 150 mM NaCl, and yeast protease inhibitors), and mixed with 10 μg ChIP-grade anti-HA (Abcam, cat #ab9110), or isotype control anti-IgG (Abcam, cat #ab171870) antibodies. Then, 50 μL of pre-washed Dynabeads Protein G for Immunoprecipitation (ThermoFisher, cat #10003D) were added to each individual sample. Chromatin immunoprecipitation was performed by incubating samples at 4° C. overnight, under gentle shaking (300 rpm). The day after, the Dynabeads were washed three times with 1 mL ice-cold wash buffer (0.1% SDS, 1% Triton X-100, 2 mM EDTA, 20 mM Tris-HCl pH 8, 150 mM NaCl), and then once with 1 mL ice-cold final wash buffer (0.1% SDS, 1% Triton X-100, 2 mM EDTA pH 8, 20 mM Tris-HCl pH 8, 500 mM NaCl), using a magnetic rack. DNA was eluted with 120 μL elution buffer (1% SDS, 100 mM NaHCO₃), by incubating samples at 30° C. for 15 min. For input, 10 μL of sheared cell lysates were added to 110 μL of elution buffer. Then, 4 μL proteinase K were added to the eluted DNA or inputs, and reverse-cross linking was achieved by incubating samples overnight at 65° C. After overnight incubation, phenol-chloroform extractions and EtOH precipitations were conducted to purify ChIP-DNA. The ChIP-DNA was eluted in a total volume of 200 μL ddH₂O, and then used for ChIP-qPCR or ChIP-Seq. Briefly, for ChIP-qPCR, 4 μL of eluted ChIP-DNA were mixed with 5 μL of PowerUp SYBR Green master mix (Applied Biosystems, cat #A25743), and 1 μL of the primer mix (forward+reverse; 3 μM final concentration), for a total volume of 10 μL. Two technical replicates were generated for each DNA sample from three ChIP replicates. ChIP-DNA samples were then transferred into the 384-well skirted PCR plate (Applied Biosystems, cat #4343370). The plate was tightly sealed with optical adhesive film (Applied Biosystems, cat #4311971). qPCR was conducted with a QuantStudio real-time PCR system from Applied Biosystems, using the following settings: 50° C./2 min, 95° C./10 min, 96° C./15 s/40 cycles, and 60° C./30 s. Dissociation curves were checked for each PCR product to assess the specificity of PCR amplicons. ChIP-seq tracks were visualized using the Integrative Genomics Viewer (IGV) v.2.8.10 from the Broad Institute (68). All primers used for ChIP-qPCR are described in FIG. 34.

### ChIP-Seq Library Preparation and Analyses

ChIP-Seq libraries were prepared using Swift S2 Acel reagents on a Beckman Coulter Biomek i7 liquid handling platform from approximately 1 ng of DNA according to manufacturer's protocol and 14 cycles of PCR amplification. Finished sequencing libraries were quantified by Qubit fluorometer and Agilent TapeStation 2200. Library pooling and indexing was evaluated with shallow sequencing on an Illumina MiSeq. Subsequently, libraries were sequenced on a NovaSeq targeting 40 million 100 bp read pairs by the Molecular Biology Core facilities at Dana-Farber Cancer Institute.

ChIP-seq reads were mapped to the yeast S. cerevisiae genome (R₆₄-1-1) with bwa v.0.7.8-r455 (http://bio-bwa.sourceforge.net/) using the mem algorithm. Peaks were called with MACS2 v.2.2.7.1 [Zhang et al 2008] using input sequencing data as a control (n=2 per condition). Differential peak depth between conditions was assessed using the R package DiffBind v.2.16.2 (https://bioconductor.org/packages/release/bioc/html/DiffBind.html). Genomic locations (yeast strain S288C) of all ORFs were downloaded from the Saccharomyces Genome Database (SGD) (http://www.yeastgenome.org) in 2019. Genomic locations of the summits were obtained from “abs_summit” column in the MACS output files. We consider a gene is occupied by Sin3 in the ChIP-seq experiments, if the start of the ORF (i.e. ATG) of the gene is within 600 bp downstream of a summit of the consensus peaks from the two repeats, and if fold change DMSO ≥1.5. Noticeably, IME2 is not present in this list due to peaks positioned above 600 bp upstream from the ATG. We consider that Sin3 occupancy is reduced when (fold change DMSO)/(fold change treatment)≥1.5. The p-values (two-sided Fisher's exact test) of the overlaps (Venn diagram) and bar graphs were calculated using python function “scipy.stats.fisher_exact”. A list of URS-comprising genes was obtained by searching the MotifMap database (http://motifinap.ics.uci.edu/, November 2019) [Daily et al 2011] using UME6 motifs Harbison_61 (“TAGCCGCCS”), and M01503 (“NNNNWNGGCGGCWAHHNNNN”) at FDR cutoff 0.1. If the start of an ORF is within 600 bp downstream of the motif, the gene corresponding to the ORF is considered as an URS-containing gene. URA3 was manually added to this list since the yeast strains used in this study contain the artificial SPAL10::URA3 reporter gene. To get a list of Ume6-bound genes, we downloaded GSE110681_Ume6_ChIP-exo5.0_peaks.gff.gz, and GSE110681_Ume6_ChIP-seq_peaks.gff.gz from the NCBI GEO website under accession GSE110681 [Rossi et al 2018]. Using the genomic locations of the peaks extracted from the gff files, we identified all the genes with ORFs whose start (i.e. ATG) is located within 600 bp downstream of the peaks. To make the list more stringent, only the genes identified using both ChIP-exo5.0 and ChIP-seq gff files were considered as Ume6-bound genes. A list of 34 random genes was also generated by randomly selecting genes in the yeast genome that: 1) did not contain a URS motif in their promoter; and 2) were not bound by Ume6 according to the criteria detailed above. In this list of random genes, three genes are occupied by Sin3 according to ChIP-seq data.

### URS-Containing Genes

A list of URS-containing genes was obtained by searching the MotifMap database (http://motifinap.ics.uci.edu/, November 2019) [Daily et al 2011] using UAE6 motifs Harbison_61 (“TAGCCGCCS”), and M01503 (“NNNNWNGGCGGCWAHHNNNN”) at FDR cutoff 0.1. If the start of an ORF is within 600 bp downstream of the motif, the gene corresponding to the ORF is considered as an URS-containing gene. URA3 was manually added to this list since the yeast strains used in this study contain the artificial SPAL10::URA3 reporter gene.

### Ume6-Binding Genes

To get a list of Ume6-binding genes, GSE110681_Ume6_ChIP-exo5.0_peaks.gff.gz, and GSE110681_Ume6_ChIP-seq_jeaks.gff.gz were downloaded from the NCBI GEO website under accession GSE110681 [Rossi et al 2018]. Using the genomic locations of the peaks extracted from the gff files, all the genes were identified with ORFs whose start (i.e. ‘ATG’) is located within 600 bp downstream of the peaks. To make the list more stringent, only the genes identified using both ChIP-exo5.0 and ChIP-Seq gff files were considered as Ume6-binding genes.

DNA-Binding Motifs and Transcription Factors from MotifMap

Transcription factor (TF) motifs and their genomic locations were downloaded from MotifMap (http://motifinap.ics.uci.edu/) [Daily et al 2011] using a false discovery rate (FDR) of 0.1. TF motif-containing genes, whose transcription start sites (TSS) were within 600 bp downstream of the motif, were then identified. Only TFs with motif-containing genes ≥10 and ≤500 were considered. Overlaps between those genes and Sin3-occupied genes (within 600 bp downstream of the consensus peaks) were then calculated. TFs with 10% or less overlaps with Sin3-occupied genes were discarded. A total of 16 TFs was thus obtained, and Sin3 occupancy at the different TF motif-containing genes was checked in the different conditions (i.e. DMSO versus TSA or E6).

### Cloning Sin3, SIN3A, SIN3B, and Ume6 Constructs

Residues 500-543 of S. cerevisiae Ume6 (SID domain) were inserted into the pET28PP (N-terminal, cleavable polyHIS fusion) vector for crystallography. The S. cerevisiae Ume6 fragments (residues 516-531), tagged (N-terminus) or not with a fluorescein isothiocyanate (FITC) probe (an aminohexanoic acid (AHA) linker was inserted between FITC and the Ume6 fragment), were synthesized by the Analytical Core Facility of the Department of Physiology at Tufts University. Residues 218-305 of S. cerevisiae Sin3 (PAH1 domain) were inserted into the pMALC2XE (N-terminal, rigid MBP fusion) vector for NMR experiments. Residues 402-473 of S. cerevisiae Sin3 (PAH2 domain) were inserted into pET28PP, pMALC2XE, and pGEX6P2 (N-terminal, cleavable GST fusion) vectors for crystallography, FP, or NMR experiments. Residues 661-728 of S. cerevisiae Sin3 (PAH3 domain) were inserted into the pGEX6P2 vector for NMR experiments. Residues 117-205 of human SIN3A (PAH1 domain) were inserted into the pMALC2XE vector for NMR experiments. Residues 301-390 of human SIN3A (PAH2 domain) were inserted into the pMALC2XE vector for NMR experiments. Residues 442-531 of human SIN3A (PAH3 domain) were inserted into the pMALC2XE vector for NMR experiments. Residues 38-113 of human SIN3B (PAH1 domain) were inserted into the pMALC2XE vector for NMR experiments. Residues 155-241 of human SIN3B (PAH2 domain) were inserted into the pMALC2XE vector for NMR experiments. Residues 306-370 of human SIN3B (PAH3 domain) were inserted into the pMALC2XE vector for NMR experiments. Final plasmid sequences were deposited into GenBank per tbl2asn2 and BankIt protocols.

### Expression and Purification of Yeast Sin3 and Ume6, and Mammalian SIN3A and SIN3B Constructs

Bicistronic yeast Sin3 PAH2/Ume6-SID: a bicistronic expression construct of yeast Sin3 (residues 402-473) and Ume6 (residues 500-543) was overexpressed in E. coli BL21 (DE3) in TB medium in the presence of 50 mg/mL of kanamycin. Cells were grown at 37° C. to an OD of 0.8, cooled to 17° C., induced with 500 μM isopropyl-1-thio-D-galactopyranoside (IPTG), incubated overnight at 17° C., collected by centrifugation, and stored at −80° C. Cell pellets were lysed in buffer A (50 mM HEPES, pH 7.5, 300 mM NaCl, 10% glycerol, 20 mM Imidazole, and 7 mM mercaptoethanol) and the resulting lysate was centrifuged at 30,000 g for 40 min. Ni-NTA beads (Qiagen) were mixed with lysate supernatant for 30 min and washed with buffer A. Beads were transferred to an FPLC-compatible column and the bound protein was washed further with buffer A for 10 column volume, and eluted with buffer B (50 mM HEPES pH 7.5, 300 mM NaCl, 10% glycerol, 300 mM Imidazole, and 7 mM BME). Human rhinovirus 3C protease was added to the eluted protein and incubated at 4° C. overnight. The sample was concentrated and passed through a Superdex 200 10/300 column (GE Healthcare) in a buffer containing 20 mM HEPES, pH 7.5, 200 mM NaCl, 5% glycerol, and 1 mM TCEP. Fractions were pooled, concentrated to approximately 19 mg/mL and frozen at −80° C.

Apo yeast Sin3 PAH2 and other MBP-tagged proteins: a construct of MBP-Sin3 (residues 402-473) was overexpressed in E. coli BL21 (DE3) in TB medium in the presence of 100 mg/mL of Ampicillin. Cells were grown at 37° C. to an OD of 0.8, cooled to 17° C., induced with 500 μM isopropyl-1-thio-D-galactopyranoside, incubated overnight at 17° C., collected by centrifugation, and stored at −80° C. Cell pellets were lysed in buffer A (50 mM HEPES, pH 7.5, 500 mM NaCl, 10% glycerol, and 7 mM mercaptoethanol) and the resulting lysate was centrifuged at 30,000 g for 40 min. Amylose beads (NEB) were mixed with lysate supernatant for 1.5 h and washed with buffer A. Beads were transferred to an FPLC-compatible column and the bound protein was washed further with buffer A containing additional 1 M NaCl for 10 column volume, and eluted with buffer B (25 mM HEPES pH 7.5, 200 mM NaCl, 5% glycerol, 20 mM malotose, and 7 mM BME). The eluted sample was concentrated and passed through a Superdex 200 16/600 column (GE Healthcare) in a buffer containing 20 mM HEPES, pH 7.5, 200 mM NaCl, 5% glycerol, 0.5 mM TCEP, and 1 mM DTT. Fractions were pooled, concentrated to approximately 20 mg/mL and frozen at −80° C. Other MBP-tagged proteins were expressed and purified following the same protocol.

GST-tagged proteins: all GST-tagged constructs were overexpressed in E. coli BL21 (DE3) in TB medium in the presence of 100 mg/mL of Ampicillin. Cells were grown at 37° C. to an OD of 0.8, cooled to 17° C., induced with 500 μM isopropyl-1-thio-D-galactopyranoside, incubated overnight at 17° C., collected by centrifugation, and stored at −80° C. Cell pellets were lysed in buffer A (25 mM HEPES, pH 7.5, 200 mM NaCl, 5% glycerol, and 7 mM mercaptoethanol) and the resulting lysate was centrifuged at 30,000 g for 40 min. Glutathione beads (GE Healthcare) were mixed with lysate supernatant for 1.5 h and washed with buffer A. Beads were transferred to an FPLC-compatible column and the bound protein was washed further with buffer A containing additional 1 M NaCl for 10 column volume, and eluted with buffer B (25 mM HEPES pH 7.5, 200 mM NaCl, 5% glycerol, 20 mM glutathione, and 7 mM BME). The eluted sample was concentrated and passed through a Superdex 200 16/600 column (GE Healthcare) in a buffer containing 20 mM HEPES, pH 7.5, 200 mM NaCl, 5% glycerol, 0.5 mM TCEP, and 1 mM DTT. Fractions were pooled, GST-tag was cleaved by incubating with GST-HRV3C protease overnight in cold room. Then, cleaved GST and GST-3C were removed by passing through GSH beads, and cleaved proteins were concentrated to approximately 17 mg/mL and frozen at −80° C.

Expression of mouse ¹³C/¹⁵N SIN3B PAH2: the same M. musculus SIN3B PAH2 C241A (residues 148-252) expressing plasmid as reported by [Kumar et al 2011] was used here. The construct was overexpressed in E. coli BL21 (DE3) in 1 L of minimum medium supplemented with 1 g 15N-ammonium chloride (Cambridge Isotope Laboratories) and 2 g 13C-glucose (Cambridge Isotope Laboratories). Cells were grown at 37° C. to an OD of 0.8, cooled to 17° C., induced with 500 μM isopropyl-1-thio-D-galactopyranoside, incubated overnight at 17° C., collected by centrifugation, and stored at −80° C. 13C/15N-double labeled protein was purified as for other GST-fused constructs. The GST tag was cleaved as described above.

### Fluorescence Polarization Assays

Dissociation constant (K_{D}) of the Sin3 PAH2/Ume6 SID interaction: 30 nM FITC-Ume6 peptide in FP buffer (10.6 mM Na₂HPO₄, 1.93 mM NaH₂PO₄, 0.5 mM EDTA, 0.01% NaN₃, pH 7.6) were prepared in Corning 3575 384-well plates, to establish the dissociation constant (K_{D}) of the GST-Sin3 PAH2/FITC-Ume6 SID interaction. GST-Sin3 PAH2 was titrated to the FITC-Ume6 SID peptide starting at a concentration of 300 μM, and followed by two-fold dilutions for a total of 24 points.

IC₅₀ determination for unlabeled Ume6 SID, E6, E6R and E6S: the concentrations of FITC-Ume6 SID (30 nM), and GST-Sin3 PAH2 (3 μM) were fixed and the compounds were titrated with an HP D300 (Hewlett-Packard, CA) to the assay plate. The assayed concentrations are given here as μM concentrations: 6, 6.6, 8.6, 11.2, 14.7, 19.3, 25.3, 33.1, 43.4, 56.9, 74.5, 97.7, 128, 167.7, 219.7, 287.9, 377.2, 494.2, 647.5 and 848.3. When compounds did not completely displace the FITC-Ume6 SID peptide from GST-Sin3 PAH2, the lowest FP values were set as the asymptote during curve fitting and IC₅₀ calculations.

### Synthesis of Enantiomers E6R and E6S

Synthesis of Ethyl 4-hydroxy-2-oxo-3,4-dihydro-2H-pyrido[1,2-a]pyrimidine-3-carboxylate (1): 2-aminopyridine (1038.2 mg, 11.0 mmol) and triethylmethanetricarboxylate (5000 mg, 21.5 mmol) were combined and dissolved in xylene (12 mL). The reaction was then heated to reflux for 90 min. Hexane (60 mL) was then added and the mixture was stirred at room temperature for 2 h. The reaction was then filtered, and the precipitate was collected. The precipitate was then treated with boiling water, filtered and the resulting filtrate was collected and concentrated to afford the desired product (1067.5 mg, 41.3%). LC-MS (ESI) m/z 235.18 [M⁺H]⁺; calcd for C₁₁H₁₁N₂O₄⁺: 235.07.

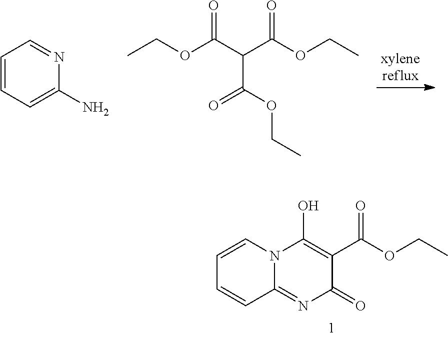 [SMILES: Nc1ccccn1 ; CCOC(=O)C(C(=O)OCC)C(=O)OCC ; CCOC(=O)c1c(O)n2ccccc2nc1=O]

Synthesis of 4-hydroxy-2-oxo-N—((R)-1-phenylethyl)-3,4-dihydro-2H-pyrido[, 2-a]pyrimidine-3-carboxamide (E6R): (R)-(+)-1-phenylethylamine (1718.4 uL, 13.5 mmol) was added to a solution of 1 (1056.9 mg, 4.5 mmol) in EtOH (8 mL). The reaction was heated to reflux for 30 h and then removed from heat and cooled to room temperature. The reaction was then filtered, and the resulting precipitate was recrystallized in EtOH to afford the desired product (966.3 mg, 69%). LC-MS (ESI) m/z 309.67 [M⁺H]⁺; calcd for C₁₇H₁₇N₃O₃⁺: 310.12. 1H NMR (500 MHz, DMSO-d6) δ 9.97 (d, J=7.8 Hz, 1H), 9.01-8.87 (m, 1H), 8.09 (ddd, J=8.7, 6.8, 1.6 Hz, 1H), 7.54 (dt, J=8.9, 1.1 Hz, 1H), 7.44-7.34 (m, 5H), 7.31-7.25 (m, 1H), 5.19 (p, J=7.1 Hz, 1H), 1.54 (d, J=6.9 Hz, 3H).

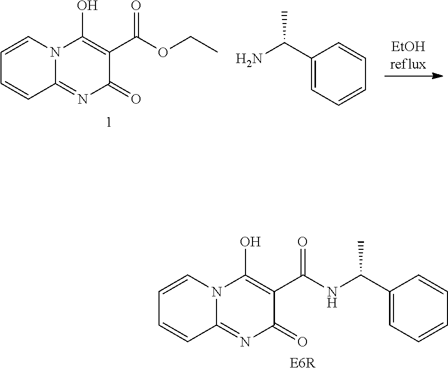

Synthesis of 4-hydroxy-2-oxo-N—((S)-1-phenylethyl)-3,4-dihydro-2H-pyrido[1,2-a]pyrimidine-3-carboxamide (E6S): (S)-(−)-1-phenylethylamine (380 μL, 3.0 mmol) was added to a solution of 1 (470.0 mg, 2 mmol) in EtOH (4 mL). The reaction was heated to reflux for 30 h and then removed from heat and cooled to room temperature. The reaction was then filtered, and the resulting precipitate was recrystallized in EtOH to afford the desired product (400.0 mg, 65%). LC-MS (ESI) m/z 309.67 [M⁺H]⁺; calcd for C₁₇H₁₇N₃O₃⁺: 310.12.

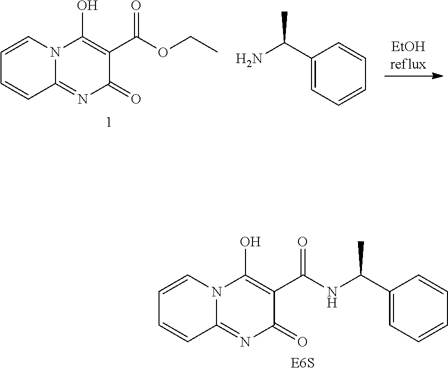

### Isothermal Titration Calorimetry (ITC)

All calorimetric experiments were carried out using an Affinity ITC from TA Instruments (New Castle, DE) equipped with auto sampler in a buffer containing 20 mM HEPES, pH 7.5, 150 mM NaCl, and 0.5 mM TCEP, with 2% DMSO at 25° C. 20 μM Sin3 solution in the calorimetric cell was titrated with 200 μM Ume6 solution using 2.5 μL injection in 200 s intervals using stirring speed at 125 rpm. Resulting isotherm was fitted with a single site model to yield thermodynamic parameters of ΔH, ΔS, stoichiometry, and K_{D} using NanoAnalyze software (TA instruments).

### Co-Crystallization of the Sin3 PAH2/Ume6 SID Interaction

With the aid of Formulatrix NT8 and RockImager and ArtRobbins Phoenix liquid handlers, a sample of 400 μM Sin3 PAH2/Ume6 SID protein and 5 μM trypsin was mixed and co-crystallized in an equivalent volume of 1.2 M NaCitrate and 0.1 M TrisHCl pH 8.0 by sitting-drop vapor diffusion at 20° C. after three days.

### Crystallization of the Apo MBP-Sin3 PAH2 Domain

A sample of 400 μM protein and 5 mM Maltose was co-crystallized in (NH₄)₂SO₄ and 0.1 M BisTris pH 6.0 by hanging-drop vapor diffusion at 20° C. using a combination of Formulatrix NT8 and ArtRobbins Phoenix liquid handlers and visualized using a Formulatrix RockImager. Large, single crystals were transferred briefly into crystallization buffer containing 25% glycerol prior to flash-freezing in liquid nitrogen and shipped to the synchrotron for data-collection.

### Data Collections and Structure Determinations

Bicistronic yeast Sin3 PAH2/Ume6-SID (6XAW): diffraction data from Sin3-Ume6 complex crystals were collected at beamline 241D-C of the NE-CAT at the Advanced Photon Source (Argonne National Laboratory). Data sets were integrated and scaled using XDS [Kabsch 2010]. Structures were solved by Br-SAD using the program Autosol in Phenix package [Adams et al 2010]. Iterative manual model building and refinement using Phenix and Coot [Emsley & Cowtan 2004] led to a model with excellent statistics.

Apo yeast Sin3 PAH2 (6XDJ): diffraction data from MBP-Sin3 PAH2 crystals were collected at beamline 241D-E of the NE-CAT at the Advanced Photon Source (Argonne National Laboratory). Data sets were integrated and scaled using XDS [Kabsch 2010]. Structures were solved by molecular replacement using the program Phaser [McCoy et al 2007] and the search model of MBP from PDB entry 4JBZ. Iterative manual model building and refinement using Phenix [Adams et al 2010] and Coot [Emsley & Cowtan 2004] led to a model with excellent statistics.

### NMR Experiments

Ligand-detected NMR experiments: all results were acquired using a 3 mm tube at 25° C. on a Bruker 600 MHz Avance II system equipped with a CPPTCI cryoprobe (Bruker BioSpin Corp.). In all experiments, the concentration of E6R was fixed at 300 μM (from a stock of 25 mM in deuterated DMSO) and the concentration of the protein at 10 μM. The volume of deuterated DMSO was kept constant (1.5%) in all tested samples. Right before the experiments, the compound and protein were diluted in 1×PBS containing 0.1% deuterated dithiothreitol (DTT) to a total volume of 200 μL. Samples were diluted by 5% with D₂O for magnetic field lock. Spectra were acquired for ligand only, protein only, and ligand+protein. Samples also contained residual protein buffers: 10.6 mM Na₂HPO₄, 1.93 mM NaH₂PO₄, 0.5 mM EDTA, 0.01% NaN₃ (pH 7.6) for yeast GST-Sin3 PAH2; 20 mM HEPES-pH 7.5, 200 mM NaCl, 5% glycerol, 40 mM maltose, 0.5 mM TCEP, 1 mM DTT for human MBP-SIN3A PAH2; and 20 mM HEPES-pH 7.5, 200 mM NaCl, 5% glycerol, 0.5 mM TCEP, 1 mM DTT for human MBP-SIN3B PAH2. STD experiments [Mayer et al 2001] were acquired with 160 scans, with protein irradiation applied for 3 s at 0 ppm and −20 ppm for the on and off resonance experiments, respectively. On and off resonance spectra were collected and stored separately. One-dimensional CPMG-R₂ edited experiments [Hajduk et al 1997] were collected for samples with and without proteins using seven CPMG delays (0, 25, 50, 100, 300, 500, and 800 ms) with four scans per delay. Proton spectra used to measure DLB and chemical shift perturbations in the absence and presence of the proteins were recorded with 16 scans using a recycle delay of 3 s. TopSpin3.2 software (Bruker BioSpin Corp.) was used to acquire and process the data. Data analysis was performed using Matlab (MathWorks, Inc.).

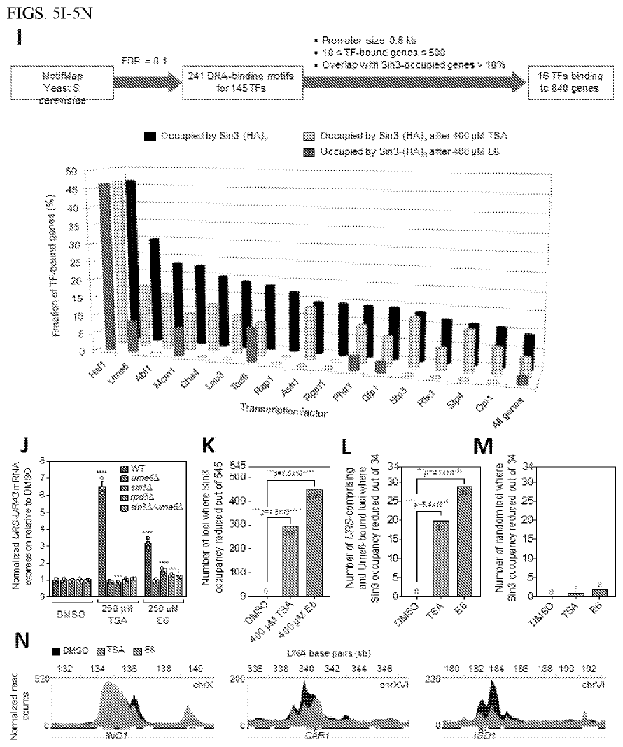

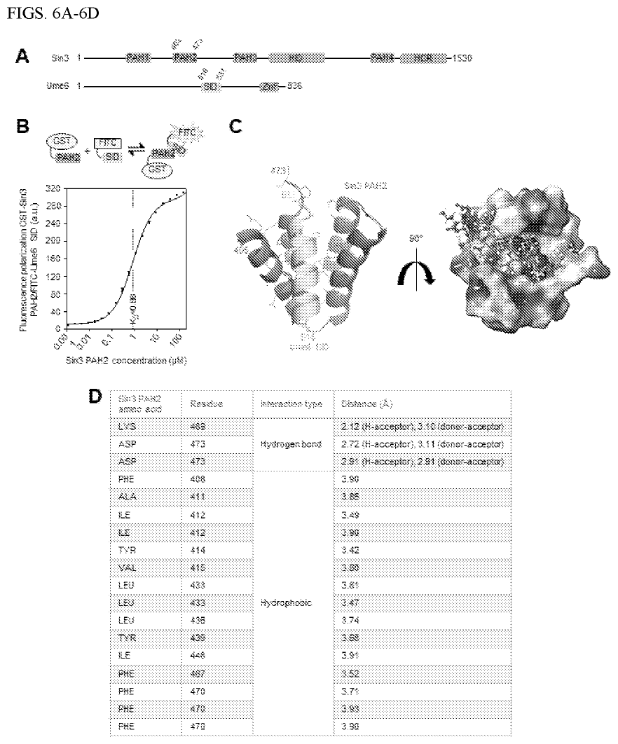

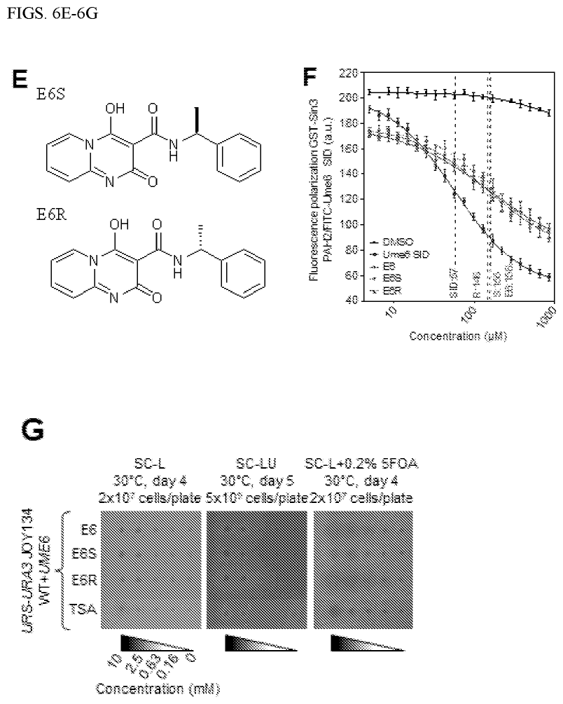

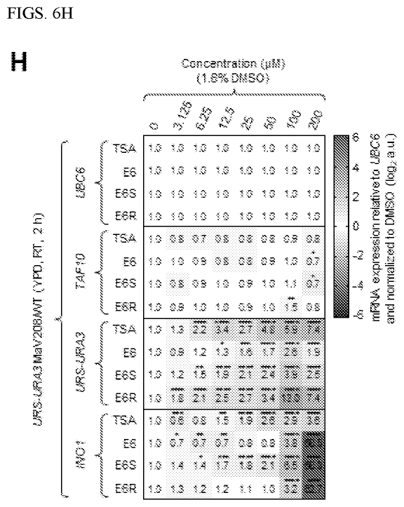

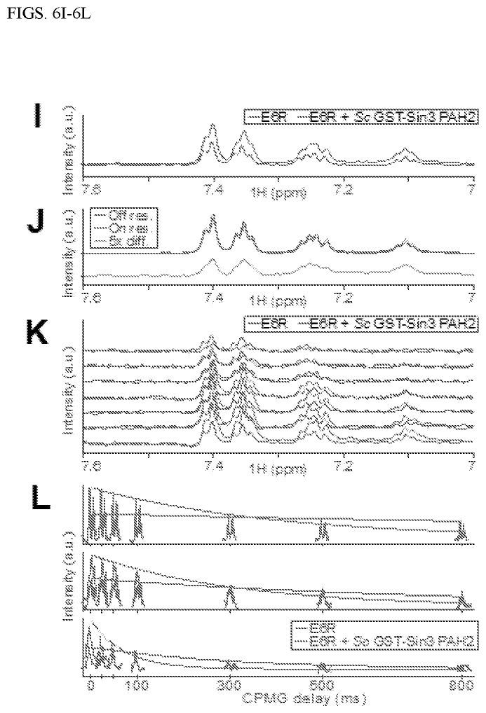

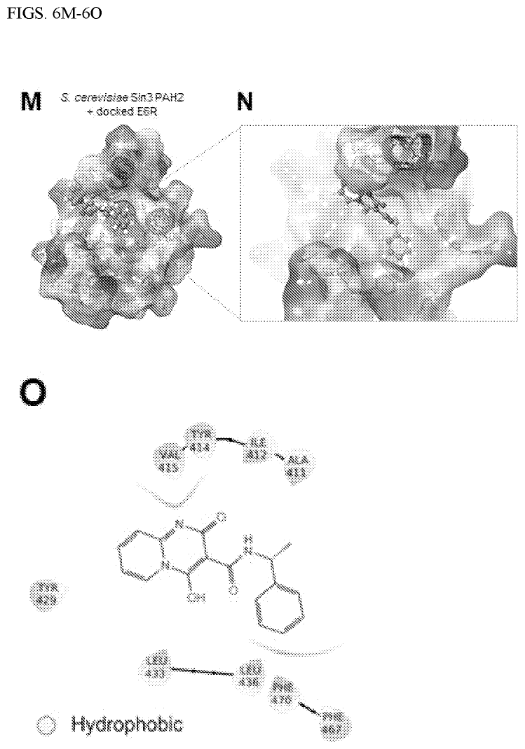

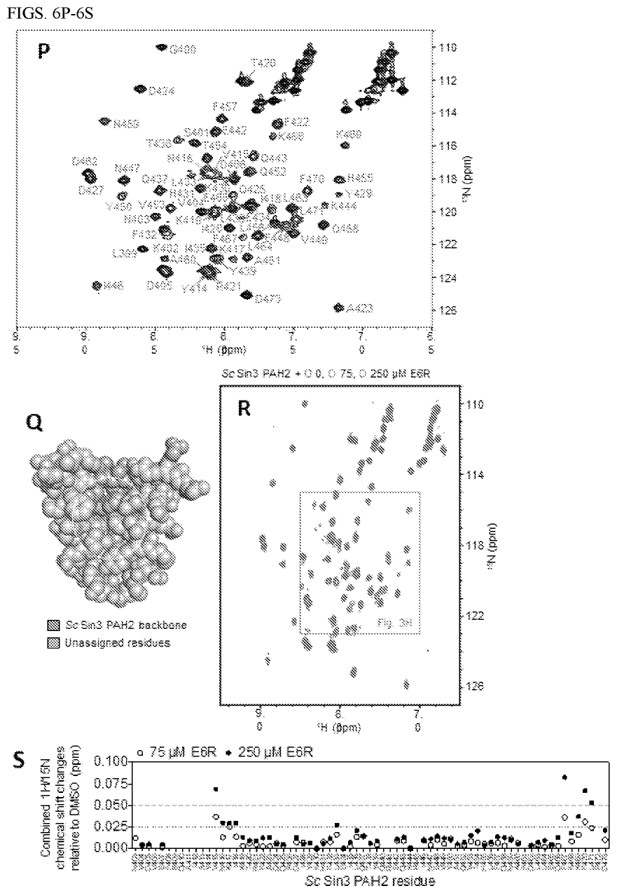

Ligand titration experiments: ¹⁵N-SOFAST HMQC spectra were acquired for 25 μM ¹⁵N-labeled yeast Sin3 PAH2 protein sample in the absence and presence of 75 μM or 250 μM E6R (diluted from 25 mM stock but with total deuterated DMSO concentration of each NMR sample kept constant at 2%). The samples also contained 20 mM HEPES-pH 7.5, 200 mM NaCl, 3 mM DTT, 0.5 mM TCEP, and 10% D₂O. Experiments were performed at 20° C. by non-uniform sampling on a Bruker Avance 11 600 MHz spectrometer equipped with a CPPTCI cryoprobe (Bruker BioSpin Corp.). The relaxation delay was 0.2 s and the number of scans was 64. Indirect ¹⁵N-dimension had a maximum of 128 complex points and 50% non-uniform sampling rate. Combined chemical shift changes in FIG. 6S correspond to “sqrt((DeltaHcs)²+(DeltaNcs/5)²)”. A TopSpin3.2 software (Bruker BioSpin Corp.) was used to acquire and process data.

NMR backbone assignments: non-uniformly sampled HNCA, HNCOCA, HNCO, HNCACO, HNCACB, CBCACONH and ¹⁵N-NOESY experiments were performed at 20° C. on a Bruker NEO 600 MHz spectrometer equipped with a cryogenic probe using the TopSpin4.1 software (Bruker BioSpin Corp.). Two 300 μM ¹⁵N-¹³C doubly labeled yeast Sin3 PAH2 domain samples were used to minimize the effect from sample degradation. The sample conditions were the same as those used for ligand titration experiments. The data were processed using the NMRPipe [Delaglio et al 1995] and hmsIST [Hyberts et al 2012] software, and analyzed with the CARA program [Keller et al 2005]. The secondary structure of the Sin3 PAH2 domain in solution was analyzed with the talosN software [Shen et al 2013].

Docking E6R into Yeast Sin3 PAH2 and Mouse SIN3B PAH2

AutoDock Vina with rigid receptor structures was used to dock E6R in the free PAH2 domain of yeast S. cerevisiae Sin3 (structure experimentally determined in this study), restricted to the Ume6 peptide-binding region [Trott & Olson 2010]. AutodockTools was used to prepare the dockings [Sanner 1999, Morris et al 2009]. For the mouse M. musculus SIN3A PAH2 model, the 30 free NMR structures/conformations available on PDB (2F05) were interrogated in the docking (ensemble docking) using QuickVina W. For the mouse SIN3B PAH2 model, the 20 NMR structures/conformations available on PDB (2L9S) were interrogated using QuickVina W. In both cases, blind docking against the entire mouse domain was conducted, and the best docking poses of the entire ensembles were examined. The same docking pose was obtained in multiple conformations of the NMR ensembles. Exhaustiveness was set to 100, and ten replicates for each ligand/receptor structure pair were acquired. Multiple sequence alignments of human, mouse and yeast S. cerevisiae SIN3 PAH2 domains were generated with Jalview Version 2 (89). Residues conserved between two, but not three, species were annotated as semi-conserved.

### Culture of SK-N-BE(2)-C Neuroblastoma Cells, RNA Extraction and Compound Treatments

SK-N-BE(2)-C cells were maintained in cell culture flasks supplemented with 10% Fetal bovine serum (FBS) and penicillin/streptomycin (PS) in Dulbecco's Modified Eagle's Medium (DMEM). The cells were maintained at a log phase in a humidified 5% CO₂ incubator at 37° C. For testing the effect of the compounds on gene expression, approximately 0.1 million cells were seeded on each well of 24-well cell culture plates, with 1 mL 10% FBS+PS+DMEM. 24 h after seeding the cells, they were exposed to the compounds or DMSO control for 16 h in a humidified 5% CO₂ incubator at 37° C., before harvesting them.

RNA Extraction from SK-BE(2)-C Neuroblastoma Cells

Cells were harvested for RNA extraction using Nucleospin 96 RNA (Macherey-Nagel, cat #740709.4) following manufacturer's protocol. Briefly, supernatant was removed, and the cells were gently washed with 1× phosphate-buffered saline. 300 μL lysis buffer were added to each well of 24 well cell culture plates. Roughly 0.1 million cells were used for harvesting each RNA sample. RNA concentration was measured for all purified RNA samples using a nanodrop spectrophotometer.

### RNA Sequencing for Yeast and Human Cell Samples

Following identical treatments as for RT-qPCR measurements, RNA was extracted as described above for yeast and neuroblastoma cells. Quality of the samples was checked by RT-qPCR before processing to RNA-Seq. RNA-Seq libraries for yeast (n=3 per condition) and neuroblastoma cells (n=3 per condition) were prepared according to the TruSeq stranded mRNA protocol (Illumina) and 101-bp single-end reads produced on an Illumina NovaSeq 6000 instrument. Reads were mapped to the human GRCh38 transcriptome (Ensembl cDNA release 97) or S. cerevisiae R64-1-1 (Ensembl cDNA release 97) and quantified using Salmon v0.8.2 [Patro et al 2017]. Read counts were summed to the gene level using tximport [Soneson et al 2015] and differential expression was assessed using DESeq2 [Love et al 2014]. Heatmaps were generated in R using the pheatmap package. The p-values of the overlaps were determined using two-sided Fisher's exact test and calculated using python function scipy.stats.fisher_exact.

### Extracting Published Transcriptomic Profiles for Yeast Sin3Δ and Rpd3Δ

Gene expression profiles, obtained by ChIP on chip experiments, for BY4742 sin3Δ, or rpd3Δ yeast strains were extracted from the Kemmeren et al study (“deleteome”): http://deleteome.holstegelab.nl/ [Kemmeren et al 2014]. The same thresholds as those used to analyze RNA-Seq results in this work were applied: |fold change|>2, adjusted p-value<0.05. The p-values of the overlaps between the two studies were determined using two-sided Fisher's exact test and calculated using python function scipy.stats.fisher_exact.

### Cell Invasion Assay

SK-N-BE(2)C neuroblastoma cells were seeded at 5×10⁴ cells per well density in a 24-well plate. Cells were treated for five days with compounds or with DMSO control in Dulbecco's Modified Eagle Medium containing 10% fetal bovine serum, penicillin and streptomycin, in quadruplicate. On Day five, cells were changed to serum-free medium with the same compound concentrations as during the 5-day treatment, and 50,000 cells were seeded on top of a Corning BioCoat Matrigel Invasion Chamber (cat #354480). Medium with 20% FBS was added as chemoattractant to the bottom of the well. Invasion assay was performed per manufacturer instructions. After 48 h, non-invading cells were removed by scrubbing with a cotton tip swab. Cells that remained inside the matrix were stained with 1% crystal violet dye and counted by eye. Percentage of invasive cells was calculated relative to DMSO control.

### Single Cell Isolation for Human Neuroblastoma SK-N-BE(2)-C Cells

Cells were grown in DMEM media supplemented with 10% FBS+PS in a 5% CO₂ incubator at 37° C. Cells were exposed to test compounds for 16 h at 25 μM E6R, 1 μM TSA, or 1% DMSO control. Cells were harvested by incubating them in TryplE for 10 min. Cells were spun down for 5 min at 300 g, and the supernatant was removed. Cells were counted via hemocytometer to 20,000 cells and resuspended in 200 μL of media.

### Single-Cell RNA-Seq of Human Neuroblastoma SK-N-BE(2)-C Cells

High-throughput scRNA-seq was performed using the Seq-Well S³ platform as previously described [Gierahn et al 2017, Hughes et al 2020]. Briefly, 20,000 cells were applied to the surface of each Seq-Well array which had been preloaded with uniquely barcoded poly(dT) mRNA capture beads (Chemgenes). Following cell loading, arrays were washed with RPMI and then sealed with a polycarbonate membrane and placed in a 37° C. incubator for 30 min. After membrane sealing, the arrays were submerged in a lysis buffer (5 M guanidine thiocyanate, 10 mM EDTA, 0.1% β-mercaptoethanol, 0.1% Sarkosyl) for 20 min, and subsequently incubated in a 2 M NaCl hybridization buffer for 40 min to promote hybridization of the mRNA to the bead-bound capture oligos. Next, the beads were removed from the arrays, and they were resuspended in a master mix for reverse transcription containing Maxima H Minus Reverse Transcriptase and buffer, dNTPs, RNase inhibitor, a template switch oligonucleotide, and PEG for 30 min at room temperature, and overnight at 52° C. with end-over-end rotation. This was followed by exonuclease digestion and second strand synthesis as described previously (Hughes et al 2020). PCR amplification was then performed to generate whole transcriptome amplification (WTA) products, and two SPRI cleanups were carried out using AMPure XP beads at 0.6× and 0.8× volumetric ratios. Then, the eluted products were quantified using a Qubit. cDNA libraries were then created using Illumina Nextera XT DNA Library Preparation Kit. Library cleanup was performed using SPRI purification as described above and library concentration and size distribution was determined using a Qubit fluorometer and Agilent TapeStation. Libraries for each sample were sequenced on a NextSeq 500 and a Nextseq 2000 with the following read structures: read 1: 21 bp; read 2: 50 bp; and index 1: 8 bp, and read 1: 26 bp; read 2: 50 bp; and index 1: 8 bp respectively. For each library 21 bases were sequenced in read 1, containing the cell barcode (12 bp) information and a unique molecular identifier (UMII, 8 bp), whereas 50 bases were obtained for the read 2 sequence. After sequencing, reads were demultiplexed using Illumina bcl2fastq (v2.20.0.422) and the resulting FASTQs were concatenated and aligned to the human “GRCh38-2020-A” reference genome using StarSolo alignment pipeline (v2.7.9a) on Terra.

Seurat QC/UMAP Generation for scRNA-Seq Data

A total of 90,306 cells and 36,601 genes were obtained across 6 samples (three conditions: DMSO, TSA, E6R; each tested in duplicate). Subsequently, low quality cells were filtered out, including cells with fewer than 200 unique genes, greater than 9,000 unique genes, and greater than 40% mitochondrial reads, resulting in 46,751 cells and 25,468 genes. The data were then normalized, scaled by a factor of 10,000, and logarithmized. Following filtering, an initial dimensionality reduction was performed by performing PCA over the 2,000 most variable genes. We visualized cells from each array using a uniform manifold approximation and projection (UMAP) plot across 25 principal components over the highly variable genes and default parameters grouped by the treatment conditions. To identify differences between drug treatment conditions, we first obtained the differentially expressed genes between the conditions. Differentially expressed genes were determined using the FindMarkers function on Seurat with “test.use=MAST” and default parameters. For these comparisons, we looked at the genes expressed by cells treated with either TSA or E6R with respect to DMSO. The genes were then filtered for adjusted p value <0.05, and plotted as a volcano plot using EnrichedVolcano. Log fold changes of the 65 genes upregulated by both TSA and E6R and 22 genes downregulated by both TSA and E6R in the bulk RNA-seq approach were determined using the FindMarkers function on Seurat with “test.use=MAST”, “logfc.threshold=0”, and “min.pct=0”. Python was used for generating the plots.

Mice Xenografted with Human Neuroblastoma SK-N-BE(2)-C Cells

Xenografts studies were performed at the TRACE PDX platform of the Leuven Cancer Institute (LKI) at KU Leuven-UZ Leuven. Treatment experiments included 24 NMRI-Foxlnu nude mice (Taconic) implanted with human neuroblastoma cell line SK-N-BE(2)-C(2 million cells/implant/mouse). Cells, tested for a full panel of murine pathogens (Impact profile I, IDEXX) prior to engraftment, were resuspended in 50% matrigel solution in PBS for injection in the mouse right flank. When tumors reached a volume of approximately 200 mm³, mice were randomly assigned to vehicle (34% DMSO, 55% saline 0.9%, 5% ethanol, 5% kolliphor, 1% NaOH 1 M), E6R (50 mg/kg), or SAHA (20 mg/kg) treatment groups. Treatments were administered daily intraperitoneally for 10 days or until reaching human endpoint (i.e. tumor volume >1,500 mm³). Tumors were measured daily with digital calipers, and volume was estimated as V=L×W×H×(π/6) (L: length; W: width; H: height). Drug treatments did not affect mice weight. All data were analyzed using GraphPad Prism 8. Comparisons between the vehicle and treated groups were tested for statistical significance using paired t-tests.

### Mouse Liver Microsome Stability Test

Mouse liver microsome (MLM) half-life measurements were performed by Scripps Florida Pharmacology Core, according to their protocols [Lamberto et al 2017]. 1 mg/mL hepatic microsomes was used to test compounds.

### Animals

Adult male C₅₇BL/6N mice (10-12 weeks old) purchased from Taconic laboratories (Bar Arbor, ME), and C₅₇BL/6N HDAC2 cKO inhouse bred male mice (described previously in [Ibi et al 2017]) were used for the experimental groups (vehicle and E6R). For assays including HDAC2 cKO mice, homozygous HDAC2loxP/loxP mice were bred to the CaMKIIα-Cre transgenic line and used the HDAC2loxP/loxP:CaMKIIα-Cre (HDAC2 cKO) mice and control littermates for these studies. All animals were housed in groups of 3-6 in 500 cm² Plexiglas cages (Allentown, NJ) in the animal care facilities at 12 h light/dark cycle (lights on, 8:00 to 20:00) at 23° C. with food and water ad libitum. Cages contained bedding (Teklad ¼″ Corncob bedding, Envigo) and one Nestlests™ (Ancare, Bellmore, NY, USA). Animal care and experimental procedures were performed according to an Institutional Animal Care and Use Committee (IACUC) approved protocol at Virginia Commonwealth University, Richmond, Virginia, USA.

### Drug Treatment and Dissection of Mouse Tissues

For the pilot RT-qPCR experiment, in addition to the vehicle condition (ethanol 100%/kolliphor/saline 0.9% in a proportion of 1:1:14.4; for the 50 mg/kg dose, 14 μL NaOH 1M were also added), three doses (mg/kg) of E6R were tested: 5, 15, and 50, with 4 (vehicle) or 3 (E6R) animals per group. A dose of 50 mg/kg was picked to conduct further experiments. For the RNA-Seq experiment, E6R was dissolved in a vehicle solution containing saline 55% v/v, DMSO 34% v/v, ethanol 5% v/v, kolliphor EL (Sigma-Aldrich, MO, USA) 5% v/v, NaOH 1M 1% v/v. The vehicle group received the same solution. Animals were injected daily by intraperitoneal (I.P.) injection with 5 μL/g of the solution (50 mg/kg of the drug or vehicle) at 11 am for 10 days. Animal wellness was verified by daily visual inspection and weighing. No abnormal behavior or significant body mass decrease (>5%) was noted in all groups. On the 11^{th} day, animals were sacrificed by bleeding via heart puncture under carbon dioxide anesthesia. The brains were quickly removed and the bilateral frontal cortex (bregma 1.90 to 1.40 mm) was dissected followed by total RNA extraction. The spleens were also collected in the pilot RT-qPCR experiments.

RNA Extraction from Mouse Tissues

Qiagen™ RNeasy mini kits (Valencia, CA) were used following the manufacturer's protocol. Extracted RNA was further purified using Qiagen™ RNeasy Mini columns to eliminate any trace of DNA, and dissolve in nuclease-free water. Purity of the RNA preparation was determined as the 260/280 nm ratio with expected values between 2 and 2.3. Samples were stored at −80° C. before being used for RT-qPCR (pilot experiment), or shipped to the GENEWIZ company on dry ice for library construction and RNA sequencing.

### RT-qPCR in Mouse Tissues

RNA was converted to cDNA using SuperScript III (Invitrogen). RT-qPCR assays were carried out in quadruplicate as previously described [Ibi et al 2017] using a QuantStudio 6 Flex Real-Time PCR system (Applied Biosystems) and the PowerUP SYBR Green Master Mix (Applied Biosystems).

Processing RNA-Seq Data from Mouse Tissues

RNA integrity (RIN) and yield were assessed on an Agilent 2100 Bioanalyzer, and samples with RIN larger than 7 were selected. Prior to the RNA-Seq, ribosomal RNA was depleted from the samples using the RiboMinus™ Human/Mouse Transcriptome Isolation Kit (Thermo Fisher Scientific, Waltham, MA). All samples were sequenced on an Illumina Hi-Seq sequencer. ˜29 million 50 bp single-end reads per sample were obtained. Sequencing adapters were removed using Trimmomatic v.0.33 [Bolger et al 2014]. Quality control at each processing step was performed using the FastQC tool v0.11.2 (quality base calls, GC content distribution, duplicate levels, complexity level) [Andrews 2010]. The Mouse GRCm38/mm10 reference genome was obtained from UCSC Genome Browser Gateway (http://hgdownload.soe.ucsc.edu/goldenPath/mm10/bigZips/chromFa.tar.gz), and the corresponding gene annotation file was obtained from Ensemble (ftp://ftp.ensembl.org/pub/release-83/gtf/mus_musculus/Mus_musculus.GRCm38.83.gtf.gz) on Feb. 20, 2016. Only autosomes, mitochondrial, and sex chromosomes were used. Reads were aligned using the subread v.1.6.2 aligner [Liao et al 2013]. Gene counts were obtained for each sample based on the last hg38 version of Ensembl transcriptome (v.87) using the featureCounts v.1.2.6 software [Liao et al 2014]. RNA-Seq counts were preprocessed and analyzed for differential expression using the edgeR v.3.24.3 [Robinson et al 2010] R package. P-values for differentially expressed genes were corrected using a false discovery rate (FDR) multiple testing correction method [Benjamini & Hochberg 1995]. Functional enrichment analysis (GO, KEGG) was performed using enrichr [Chen et al 2013]. Enrichment analysis using custom signatures was performed using hypergeometric test in the clusterProfiler v.3.10.1 R package [Yu et al 2012]. Row-median centered log₂(TPM+1) expression profiles for selected genes were visualized using the pheatmap package v.1.0.12. All statistical calculations were performed within R/Bioconductor environment v3.5.3.

ChIP-qPCR Assays from Mouse Tissues

ChIP assays were performed using the MAGnify ChIP system (ThermoFisher, MA) according to the manufacturer's protocol with a few modifications. A small section (50 mg) of the frontal cortex (FC) was used. Chromatin was sheared with the Covaris® S2 system (Covaris, Woburn, MA) using the following program: duty cycle 5%, intensity 2, 200 cycles per burst, 60 s cycle per time, 20 cycles, temperature 4° C. A volume of 10 μL chromatin was used per sample. A total of 0.5 μg of the anti-H3 antibody (PA5 16183, Invitrogen, Carlsbad, CA), 1 μg of the anti-SIN3A antibody (ab3479, Abcam, Cambridge, MA) or anti-IgG (ab37355, Abcam) was used per sample. Quantitative real-time PCR analysis was performed following magnetic bead-based DNA purification. The DNA samples were amplified in quadruplicate reactions as previously described [Ibi et al 2017] using the QuantStudio 6 Flex Real-Time PCR System (Applied Biosystems) using the PowerUP SYBR Green Master Mix (Applied Biosystems). All primers for ChIP-qPCR are described in FIG. 34.

### Head-Twitch Response Assay

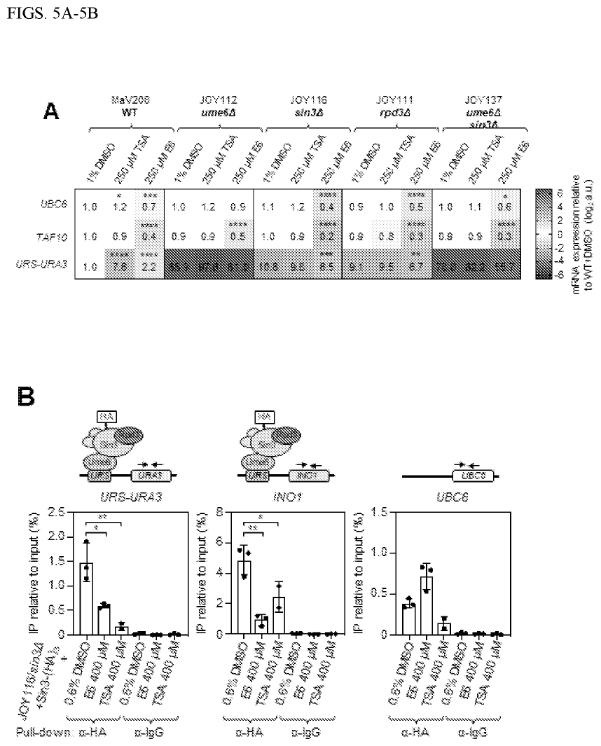

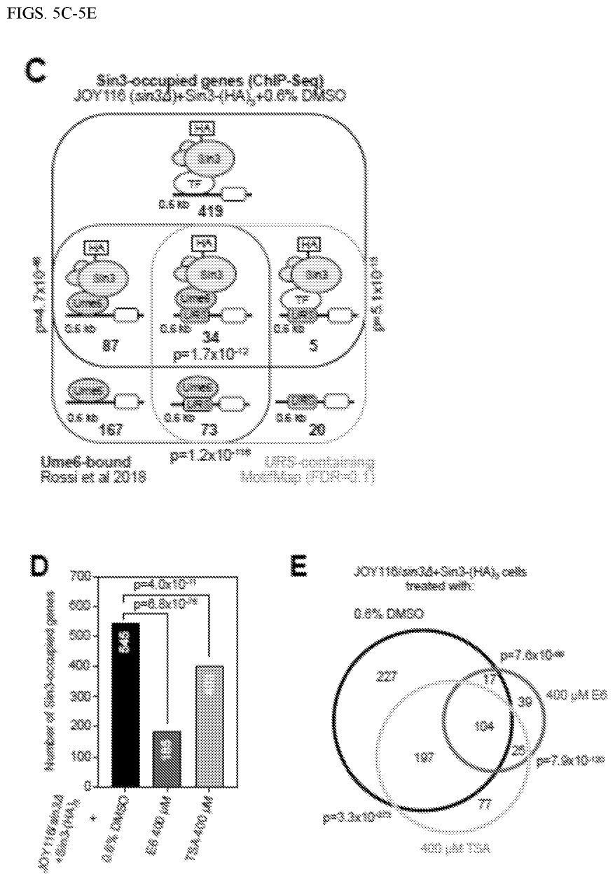

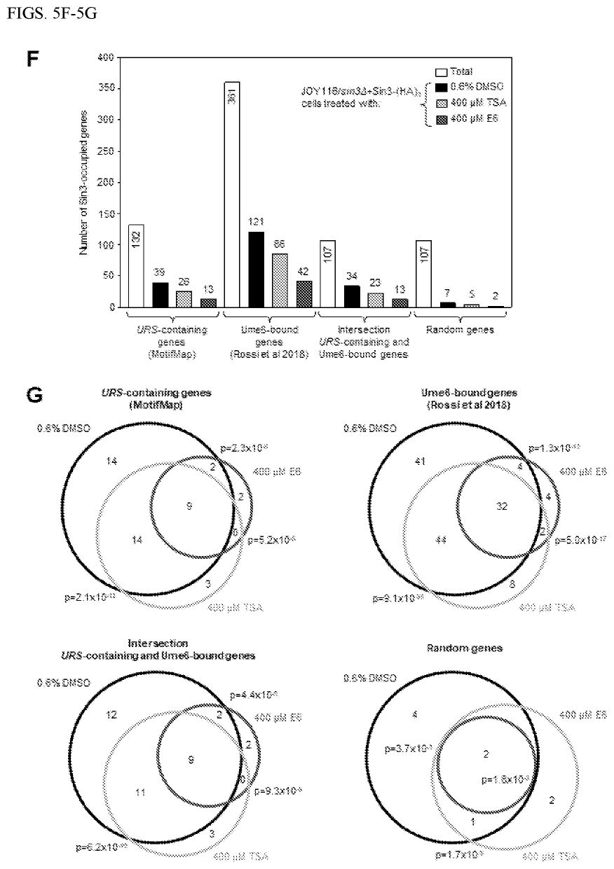

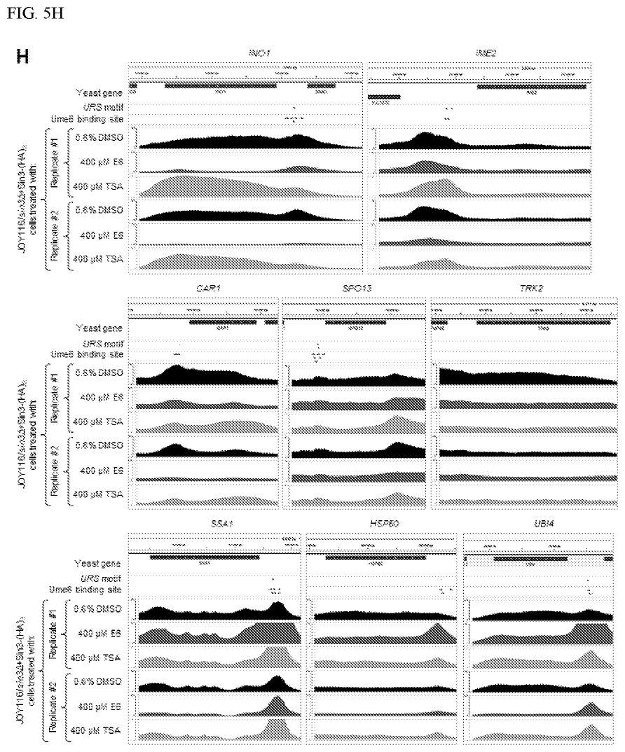

Adult male C57BL/6N mice (10-12 weeks old) purchased from Taconic laboratories (Bar Arbor, ME), and C57BL/6N Hdac2 cKO inhouse bred male mice (described previously in Ibi et al 2017) were used for the experimental groups (vehicle and E6R). For assays including Hdac2 cKO mice, we bred homozygous Hdac2loxPAoxP mice to the CaMKIIα-Cre transgenic line and used the Hdac2loxP/loxP:CaMKIIα-Cre (Hdac2 cKO) mice and control littermates. All animals were housed in groups of 5-6 in 500 cm² Plexiglas cages (Allentown, NJ) in the animal care facilities at 12 h light/dark cycle (lights on, 8:00 to 20:00) at 23° C. with food and water ad libitum. Cages contained bedding (Teklad ¼″ Corncob bedding, Envigo) and one Nestlests™ (Ancare, Bellmore, NY, USA). Animal care and experimental procedures were performed according to an Institutional Animal Care and Use Committee (IACUC) approved protocol at Virginia Commonwealth University, Richmond, VA, USA. For automated recording of HTR, WT and Hdac2 cKO male mice (10-12 weeks at the start of the experiment) were ear-tagged as previously described [de la Fuente Revenga et al 2020]. For the Hdac2 cKO group (n=5), an intraperitoneal injection (2 mg/kg; 10 μL/g) of the psychedelic drug (±)-2,5-dimethoxy-4-iodoamphetamine (DOI) (Sigma Aldrich) was delivered and HTR recorded and quantified. All mice were allowed to recover for 7 days before starting the chronic treatments. After this washout period of 7 days, chronic treatments over 10 days (daily intraperitoneal injections around the same time: 5 μL/g) was administered to Hdac2 cKO and a group of WT mice. In parallel, another group of WT mice were chronically administered with vehicle (34% DMSO, 55% saline 0.9%, 5% ethanol, 5% kolliphor, 1% NaOH 1 M), or 50 mg/kg E6R (fresh powder dissolved into the vehicle solution). 24 h after the last injection, DOI (2 mg/kg; 10 μL/g) was injected and HTR subsequently recorded and quantified. For each session, the animals were habituated to the testing chamber prior to DOI administration. Mice were then returned to the chamber right after DOI administration. HTR were recorded during both habituation and post-DOI administration for 30 min and quantified in 15 min bins. Data were processed as previously described [de la Fuente Revenga et al 2013, de la Fuente Revenga et al 2020]. Potential false positives and false negatives were visually inspected during the signal processing. Results presented in FIGS. 5B-C: for WT mice, one group (n=5) was chronically treated with vehicle, and the other group (n=6) was chronically treated with 50 mg/kg E6R. 24 h after the last injection, both groups received 2 mg/kg DOI, and HTR was recorded for each group as described above. For the Hdac2 cKO group (n=5), drug naïve mice were treated with 2 mg/kg DOI. Following a washout period of 7 days, and chronic treatment with 50 mg/kg E6R (over 10 days), 2 mg/kg DOI was injected again to the same mice (24 h after the last injection of E6R), and HTR were recorded as described above. Therefore, for Hdac2 cKO animals, the effect of DOI was compared before and after chronic E6R treatment within the same group.

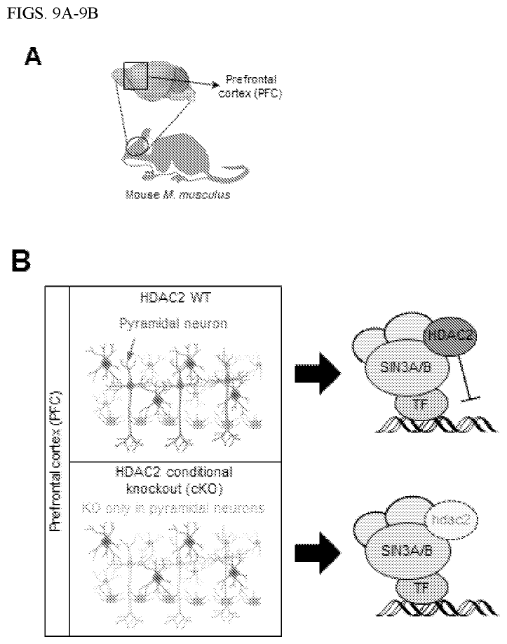

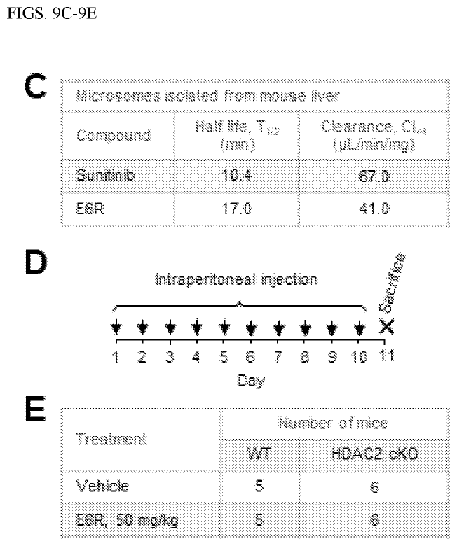

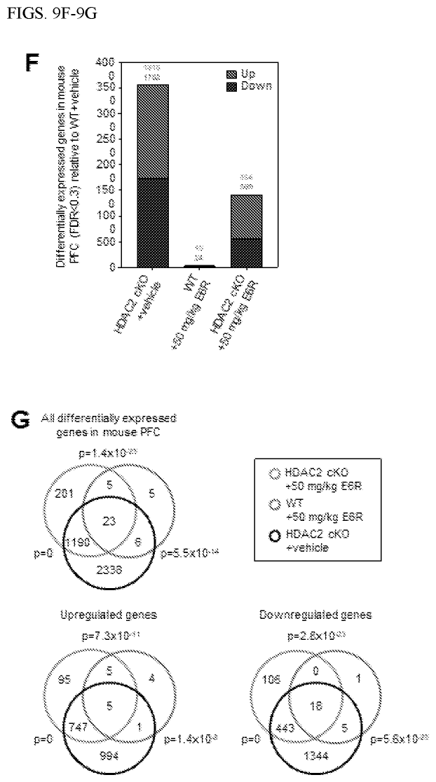

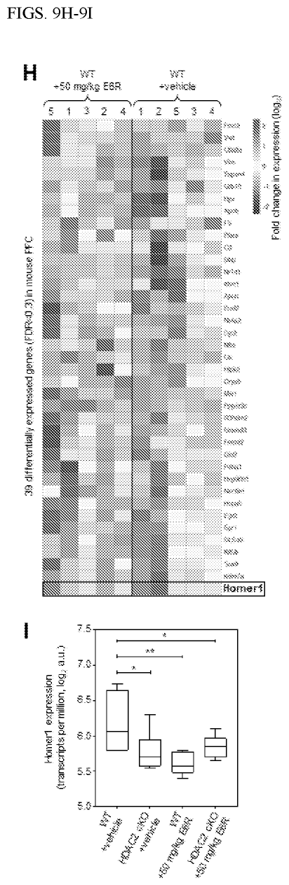

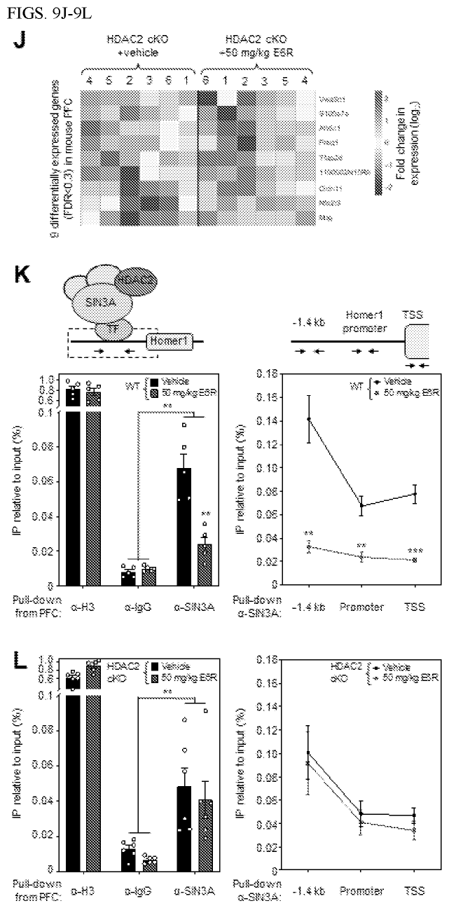

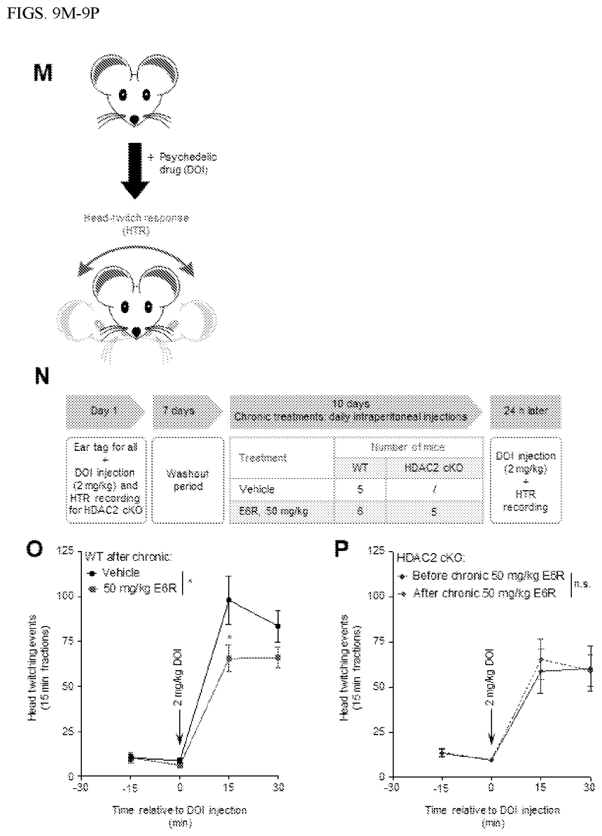

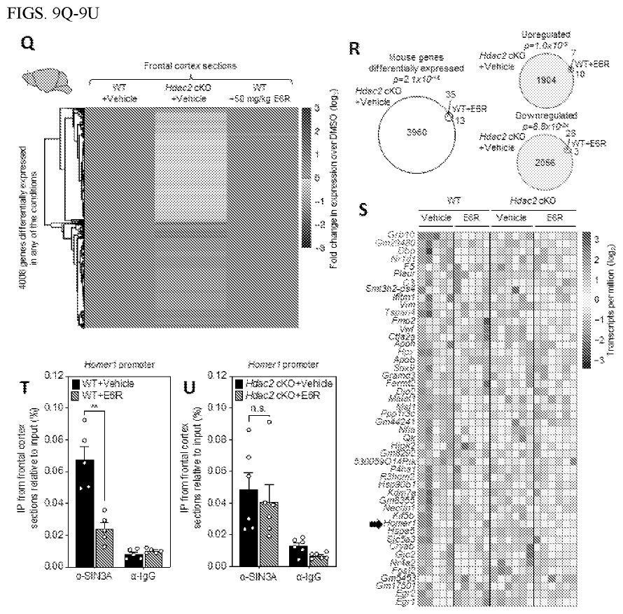

Construction of FIG. 9O-P: for WT mice, one group (n=5) was chronically treated with vehicle, and the other group (n=6) was chronically treated with 50 mg/kg E6R. 24 h after the last injection, both groups received 2 mg/kg DOI, and HTR was recorded for each group as described above. For the HDAC2 cKO group (n=5), mice were treated with 2 mg/kg DOI on day 1, after ear-tagging. Following the washout period of 7 days, and chronic treatment with 50 mg/kg E6R (over 10 days), 2 mg/kg DOI was injected (24 h after the last injection of E6R), and HTR were recorded as described above. Therefore, for HDAC2 cKO animals, the effect of DOI was compared before and after chronic E6R treatment within the same group.

### Statistics

Except otherwise indicated, the statistical calculations were performed using Prism 8.3.1., following the methods indicated in the text, materials and methods or figure legends. Log₂ values were used to calculate statistics. The number of replicates used in each experiment is indicated in the figure legends and/or in the materials and methods. Standard deviations (SD) and standard errors of the mean (SEM) were calculated as follows:

$$SD = \sqrt{\frac{{\sum }_{i = 1}^{n} {( {x}_{i} - \overline{x} )}^{2}}{n - 1}} , and SEM = \frac{SD}{\sqrt{n}} \qquad ( 2 )$$

Where “n” is the sample size, “xᵢ” the data point i, and “x̅” the mean of all data points.

### EXAMPLES

The invention is further described in the following examples, which do not limit the scope of the invention described in the claims.

### Example 1: A Combined Targeted Phenotypic Approach to Assess all Functionalities of HDAC Complexes

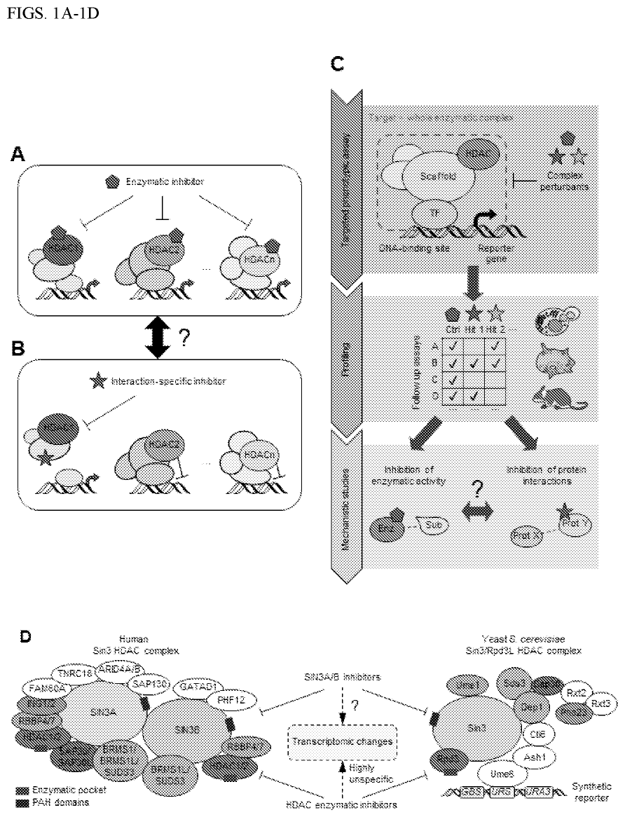

To interrogate the different functionalities of HDAC complexes with small molecules, a pipeline was developed that starts with a phenotypic assay, built by bringing a well-characterized reporter gene (e.g. auxotrophic marker or light-producing protein coding gene) downstream to the DNA-binding site of the complex (FIG. 1C). This system confers a positive selection for disruptive compounds and allows interrogating the complex in the appropriate cellular environment, where all its components (i.e. enzymatic and others) are agnostically explored. In particular, it allows the detection of alternative disrupting mechanisms such as those involving biophysical interactions, including protein-protein interactions (PPIs). Furthermore, most subunits of the selected complex are known (e.g. listed in protein interaction maps [Huttlin et al 2015, Wan et al 2015, Hein et al 2015, Luck et al 2020]), to better focus downstream target deconvolution. The next step involves assessing the transcriptional effects of the compounds against genes known to be regulated by the complex, and comparing them to classical enzymatic inhibitors. Finally, compounds are tested against the enzymatic activity of the HDAC enzyme, in parallel to being tested against binary interactions between established subunits of the complex.

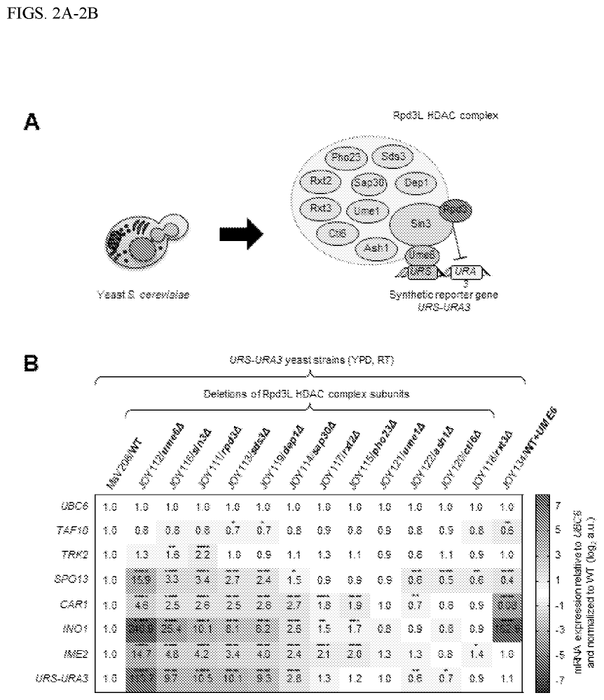

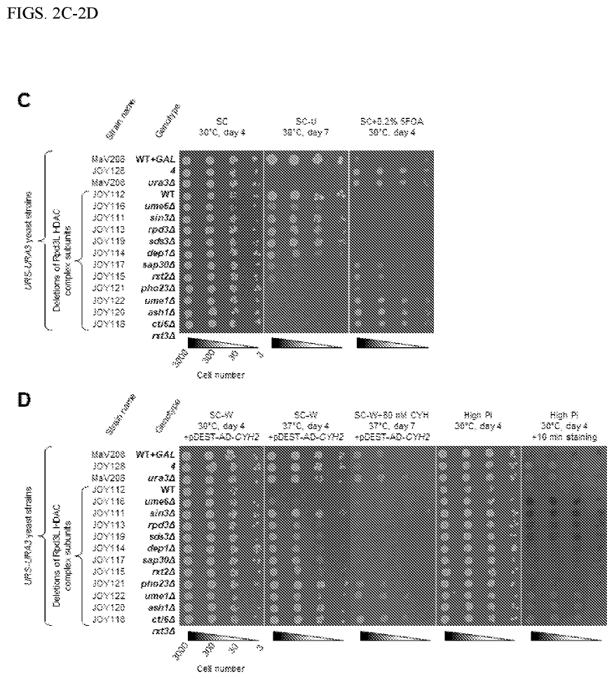

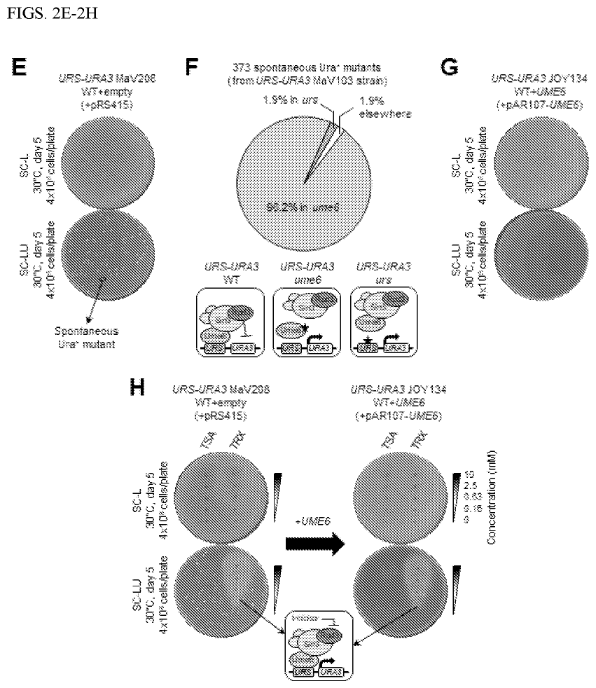

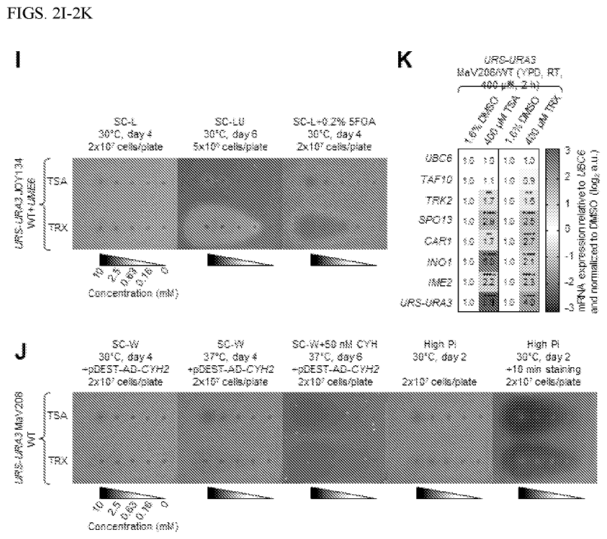

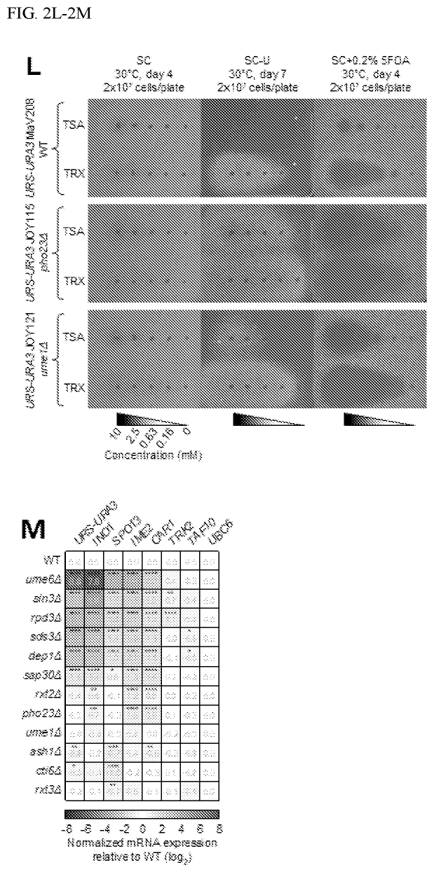

As a starting point to test our approach, a well-documented, pan-species HDAC complex was selected, around which powerful and easily implementable biochemical and genetic tools could be developed. This system originally established key fundamental aspects of HDAC biology, so that any new compound from this work could be compared without bias to canonical HDAC enzymatic inhibitors. The yeast S. cerevisiae Rpd3 Large (Rpd3L) HDAC complex (FIG. 1D) was selected, where Rpd3 is the catalytic HDAC component, Sin3 a large scaffold protein that interacts with Rpd3 among other proteins, and Ume6 a transcription factor that recruits Sin3 and binds to DNA via an upstream repressing sequence (URS), consisting of a dozen base pairs [Dubois et al 1978, Vidal & Gaber 1991, Vidal et al 1991, Stritch et al 1994, Kadosh & Struhl 1997, Washburn & Esposito 2001, Grigat et al 2012]. Over the years, many genetic and biochemical studies have explored the Rpd3L HDAC complex and 12 subunits, recapitulated in affinity purification/mass-spectrometry (AP-MS) experiments, are now commonly reported [Sardiu et al 2009]: Ume6, Sin3, Rpd3, Sds3, Dep1, Sap30, Rxt2, Pho23, Ume1, Ash1, Cti6, and Rxt3 (FIG. 2A). Most functions mediated by these subunits still remain unknown.

To evaluate the composition of the Rpd3L HDAC complex at different loci, expression of five well-established Rpd3-regulated genes, TRK2, SPO13, CAR1, IME2 and INO1 was measured, by reverse transcription-quantitative PCR (RT-qPCR) when the different subunits are individually knocked out (FIGS. 29-31). These genes are likely repressed by a Rpd3L HDAC complex as, except for TRK2, they all contain at least one URS-binding site motif in their promoter [Vidal 2016]. Expression levels were calculated relative to UBC6, a commonly used housekeeping gene, and TAF10, a gene not known to be directly regulated by Rpd3, was employed as control for specificity [Teste et al 2009]. In agreement with published observations, FIG. 2B shows that only Sin3 and Rpd3 were involved in TRK2 repression [Vidal et al 1990, Vidal & Gaber 1991]. However, six subunits were commonly found in the Rpd3L HDAC complex at other loci: Ume6, Sin3, Rpd3, Sds3, Dep1, and Sap30. Besides, their deletions gave rise to different transcriptional effects on the downstream genes. For example, deleting Ume6 derepressed INO1 by ˜247-fold, while deleting Rpd3 derepressed it by ˜10-fold. In addition to these six core subunits, Rxt2 and Pho23 also seemed to play a role in the repression of CAR1, INO1, and IME2 since their deletions increased expression of those genes. Interestingly, deleting subunits such as Ash1, Cti6, or Rxt3 decreased the basal expression levels of SPO3 and CAR1, which suggest they might be involved in transcriptional activation mechanisms.

To develop our focused, yet agnostic, phenotypic assay, synthetic SPAL10::URA3 reporter system was taken advantage of in MaV yeast strains [Vidal et al 1996], where the Rpd3L HDAC complex is recruited, via URS, in front of a uracil auxotrophic marker, URA3 (URS-URA3) (FIG. 2A). In this context, the Rpd3L HDAC complex represses URA3 and prevents expression of the Ura3 protein involved in uracil biosynthesis, ultimately blocking growth on media lacking uracil. Importantly, the composition of the complex at that locus was comparable to that of established Rpd3-regulated genes since the same six core subunits were also involved in URA3 repression (FIG. 2B). However, under these conditions, cells are able to grow on media containing 5-fluoroorotic acid (5FOA), as the absence of the Ura3 protein prevents transformation of 5FOA into a toxic product. The URS-URA3 reporter system can thus be used to apply both positive and negative selections when perturbations are introduced in the Rpd3L HDAC complex. This mirror selection is illustrated in FIG. 2C where, comparable to a strain lacking URA3 (ura3Δ), WT cells where URA3 is repressed by the Rpd3L HDAC complex (URS-URA3, WT) do not grow on media lacking uracil (Ura⁻), but grow on media containing 5FOA (Foa⁺). In short, these yeast strains display a Ura⁻/Foa⁺ phenotype. Conversely, cells where GAL4 was expressed from a plasmid (FIG. 30) to activate URA3 expression by binding to upstream Gal4-binding sites in the SPAL10::URA3 reporter (URS-URA3, WT+GAL4) exhibited a Ura⁺/Foa⁻ phenotype, i.e. growth on media lacking uracil, no growth on media supplemented with 5FOA.

To evaluate if the transcriptional profile of the Rpd3L HDAC complex at URS-URA3 could also be observed phenotypically, the impact of subunit deletions on yeast growth on media lacking uracil was measured, or supplemented with 5-fluoroorotic acid (5FOA) (FIG. 2C). Comparable to yeast cells lacking genomic URA3 (ura3Δ), wild-type (WT) cells in which URA3 is repressed by the Sin3/Rpd3L HDAC complex (URS-URA3) do not grow on media lacking uracil (i.e. Ura⁻ phenotype), but do grow on media containing 5FOA (i.e. Foa⁺ phenotype). Six subunits of the Sin3/Rpd3L HDAC complex were found to contribute to URS-URA3 repression: Ume6, Sin3, Rpd3, Sds3, Dep1, and Sap30, since they confer strong Ura⁺/Foa⁻ phenotypes similar to cells in which URA3 is ectopically induced by the Gal4 TF (WT+GAL4). Interestingly, knocking out Rxt2 or Pho23 was associated with intermediate Ura⁺/Foa⁻ phenotypes while deletions of other subunits led to Ura⁻/Foa⁺ phenotypes, indicating that they are not required for repression at the URS-URA3 locus.

To further generalize observations from FIG. 2B, the effect of subunit deletions was benchmarked using two well-established Rpd3-sensitive phenotypes: cycloheximide (CYH) sensitivity (Cyh^{S}) at 37° C., and acid phosphatase activity [Vidal et al 1990, Vidal & Gaber 1991]. Indeed, if knocking out Rpd3 gives rise to particular phenotypes, deleting proteins involved in the same complexes or pathways than Rpd3 might also lead to comparable results. First, the intrinsic CYH resistance (Cyh^{R}) phenotype of MaV208, and associated subunit-lacking strains, was overcome by transforming cells with a vector carrying the CYH2 counter-selectable marker (FIG. 30). As depicted in FIG. 2D, the growth pattern on media supplemented with CYH was comparable to that on 5FOA-containing media, with the exception of cti6Δ, which gave rise to Cyh^{S}, likely indicating the involvement of this subunit in the cellular response to this chemical. Noticeably, among the 12 subunits tested, only ume6Δ and sin3Δ displayed growth defects when incubated at 37° C. instead of 30° C. In agreement with published studies [Vogelauer et al 2000], FIG. 2D also shows that Ume6, a known URS-binding TF, does not seem to recruit the Rpd3L HDAC complex at genes regulating acid phosphatase activity as its deletion, similar to cti6Δ, rxt3Δ or ash1Δ, did not lead to a red coloring on phosphate-rich (high Pi) media. Alongside FIG. 2B, these phenotypic results highlight the various compositions the Rpd3L HDAC complex can adopt to regulate genes involved in different cellular pathways. Altogether, those findings demonstrate that even if the catalytic Rpd3 subunit appears to always be part of the complex, other usually reported members are also required to ensure its proper function at different loci.

To check if the Ura⁺ phenotype to be used to identify new compounds could indeed be linked to perturbation events in the Rpd3L HDAC complex at the URS-URA3 locus, spontaneous Ura⁺ mutants were characterized that grow at a frequency of one per 1×10⁶ when wild-type, haploid yeast cells are seeded on media lacking uracil. Indeed, haploid MaV strains containing the URS-URA3 reporter system (i.e. SPAL10::URA3), like MaV208, spontaneously give rise to such Ura⁺ mutants when plated on uracil dropout media (FIG. 2E). In these conditions, the only ways for wild-type URS-URA3 cells to grow are by circumventing URA3 repression to induce uracil production; one of the apparent solutions being to force the Rpd3L HDAC complex to dysfunction at that locus. 373 spontaneous URS-URA3 Ura⁺ mutants were picked and obtained using the MaV103 (MATa) strain and have mated them against wild-type MaV203 (MATa), and associated subunit-lacking strains. Among these, 366 mutants (98.1%) were complemented by the wild-type MaV203 strain naturally harboring a second copy of every subunit, as they recovered a Ura⁻ phenotype. Within this group, 359 mutants (96.2%) were also complemented by the deletion strains, except by ume6Δ, suggesting nearly all mutations likely lie within UME6, i.e. within the gene coding for the DNA-binding protein recruiting the Rpd3L HDAC complex at URS-URA3 (FIG. 2F). However, out of the 373 Ura⁺ mutants, seven (1.9%) failed to be complemented by the wild-type strain and maintained a Ura⁺ phenotype. These were all confirmed by sequencing to have mutations in URS; i.e. in the ˜10 base pairs Ume6-DNA binding site at URS-URA3 (data not shown).

Finally, to decrease the number of spontaneous Ura⁺ mutants and maximize the signal-to-noise ratio for further chemical screens, the number of UME6 copies in wild-type URS-URA3 MaV208 yeast cells were increased. This was achieved by transforming them with a plasmid expressing UAE6, to obtain the JOY134 strain (FIGS. 29-30), which allowed decreasing by ˜100-fold the frequency at which spontaneous Ura⁺ mutants could grow on media lacking uracil (FIG. 2G). Noticeably, this extra copy of UME6 in the MaV208 background did not modify URA3 expression at URS-URA3 (FIG. 2B). In addition to providing optimal conditions to interrogate all its functionalities with small molecule perturbants, these results show that the characterized MaV208 Ura⁺ mutants indeed arise from spontaneous disruption events in the Rpd3L HDAC complex, or in its DNA-binding site, at the URS-URA3 locus.

To measure expression levels at URS-URA3 in the different deletion mutant strains, reverse transcription-quantitative PCR (RT-qPCR) was performed. In agreement with the Ura⁺/Foa⁻ phenotypes, it was confirmed that repression of the synthetic URS-URA3 locus is relieved when deleting any of the six core subunits of the Sin3/Rpd3L HDAC complex (FIG. 2M). The profile was then compared to that of five well-established Rpd3-regulated genes, INO1, SPO13, IME2, CAR1 and TRK2 [Strich et al 1994], and observed that deleting subunits confer different transcriptional responses. For example, deleting Ume6 increased INO1 expression by ˜230-fold, while deleting Rpd3 only had a ˜10-fold effect. Rxt2 and Pho23 also altered repression of CAR1, INO1, and IME2 as their deletion led to induction of these genes. Consistent with published observations [Vidal et al 1990], only sin3Δ and rpd3Δ affected TRK2 repression. Finally, deleting Ash1, Cti6, or Rxt3 decreased expression levels of SPO3 and in some cases CAR1, suggesting their potential involvement in transcriptional activation mechanisms. Together, these results highlight the different compositions the Sin3/Rpd3L HDAC complex can adopt at different loci and support the idea that targeting components other than Rpd3 might lead to higher selectivity.

### Example 2: Benchmarking Targeted Phenotypic Assays Against Well-Established Enzymatic Inhibitors

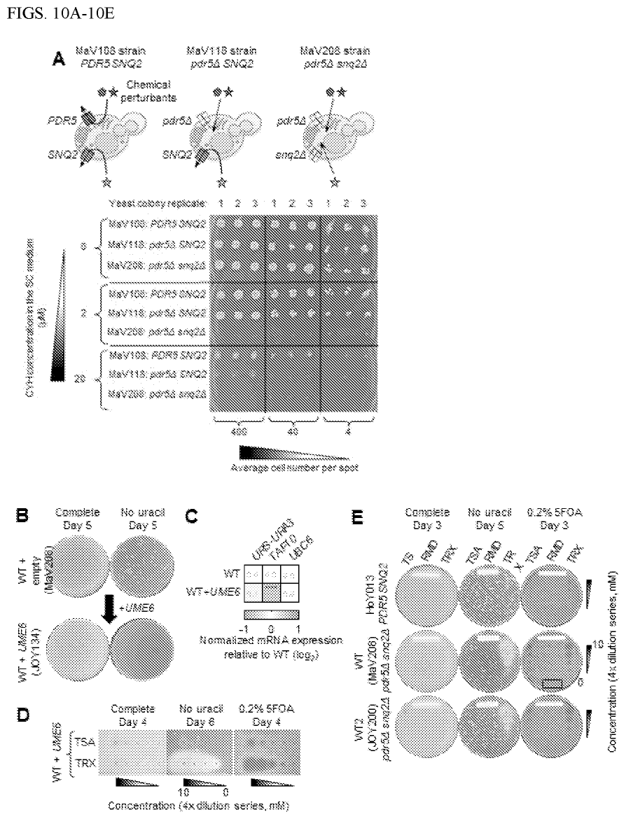

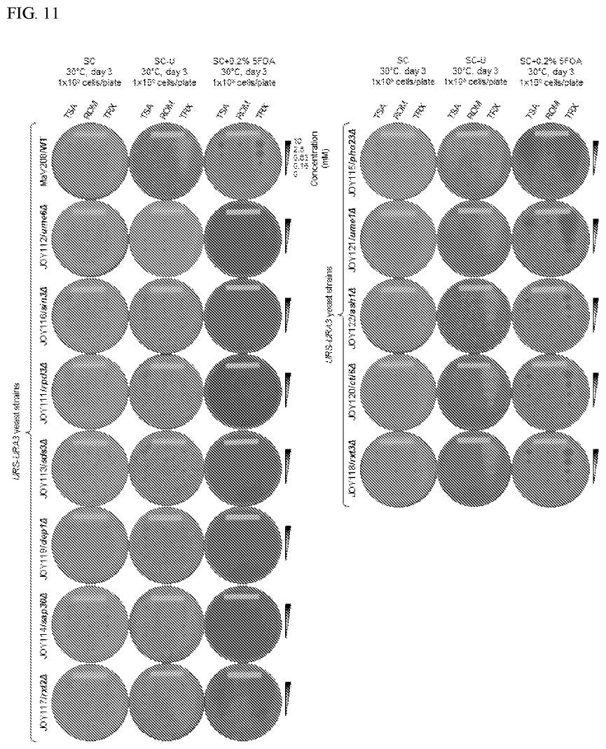

In order to compare any new compound from the screen to well-established, pioneer HDAC enzymatic inhibitors trichostatin A (TSA) and trapoxin A (TRX) in JOY134 (i.e. MaV208+UME6) were tested, URS-URA3 yeast cells where two genes coding for drug efflux pumps, PDR5 and SNQ2, are deleted. These two efflux pumps were shown to be responsible for most pleiotropic drug resistance in S. cerevisiae [Balzi & Gofeau 1995, Piecuch & Oblak 2014], and their deletions conferred drug sensitivity phenotypes that are approximately two orders of magnitude greater than for wild-type strains (FIG. 10A). To maximize signal-to-noise ratio of the URS-URA3 reporter assay, a plasmid encoding the Ume6 TF was transformed into WT haploid cells to decrease the frequency of Ura⁺ mutant colonies spontaneously growing on uracil dropout media by ˜100-fold (FIG. 10B). Notably, this extra copy of UME6 did not modify expression at URS-URA3 (FIG. 10C). Using an agar diffusion assay, it was that found that both TSA and TRX led to Ura⁺/Foa⁻ phenotypes while having little or no effect on overall cell viability on complete media (FIG. 10D). The effect was stronger for TRX than for TSA, which might be explained by their different binding modes to the catalytic pocket of HDAC enzymes [Yoshida et al 1995]. Altogether, these experiments established the relevance of the URS-URA3 reporter assay for identifying novel small molecule perturbants targeting the biological functions of the conserved Sin3/Rpd3L HDAC complex.

The design of the assay is such that wild-type URS-URA3 yeast cells are plated onto solid medium lacking uracil. Compounds are subsequently spotted onto this lawn of non-growing Ura⁻ cells, and allowed to diffuse to produce a gradient of concentrations. After penetrating cells, compounds that can perturb the Rpd3L HDAC complex will thus confer a Ura⁺ phenotype which will appear as a ring of growth around the original spot, following incubation for a couple of days (FIG. 2H). This primary positive selection for growth has obvious advantages over settings using negative readouts, such as reduction of reporter signal or cell viability. Indeed, it filters out compounds that are cytotoxic and non cell-permeable, while preventing the constraint of having to define a particular concentration for the entire screening because of the gradient that forms around the spotted compounds. This agar diffusion assay format was validated, in a dose-dependent manner, by the observation that TSA and TRX: i) gave rise to rings of growth on media lacking uracil around their respective spots; ii) prevented growth on 5FOA around the spots as well; while iii) having little or no effect at all on the overall cell viability on complete media (FIG. 2I). Interestingly, the intensity of the readouts was stronger for TRX than for TSA, which might be explained by their different binding modes to the catalytic pocket of HDACs: covalent versus reversible, respectively.

To evaluate the extent to which traditional enzymatic HDAC inhibitors such as TSA and TRX broadly perturb Rpd3-involving pathways, those compounds were yested, in a dose-dependent manner, against the same Rpd3-sensitive phenotypes presented in FIG. 2D (FIG. 2J). It was observed that TSA and TRX both phenocopy rpd3Δ as they similarly induced CYH sensitivity and acid phosphatase activity, which is in agreement with their HDAC enzymatic inhibition mechanism. Interestingly, compared to plates lacking uracil, TSA gave rise to stronger readouts on CYH-containing media, and media with high concentration of phosphate, while TRX had a similar effect throughout the different tested conditions. In addition, similar to rpd3Δ, TSA and TRX both increased expression of well-established Rpd3-regulated genes by ˜1.5 to 9-fold (FIG. 2K).

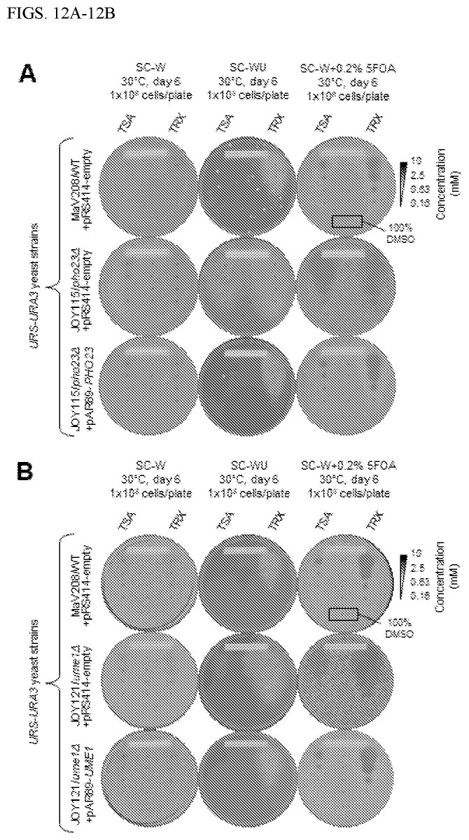

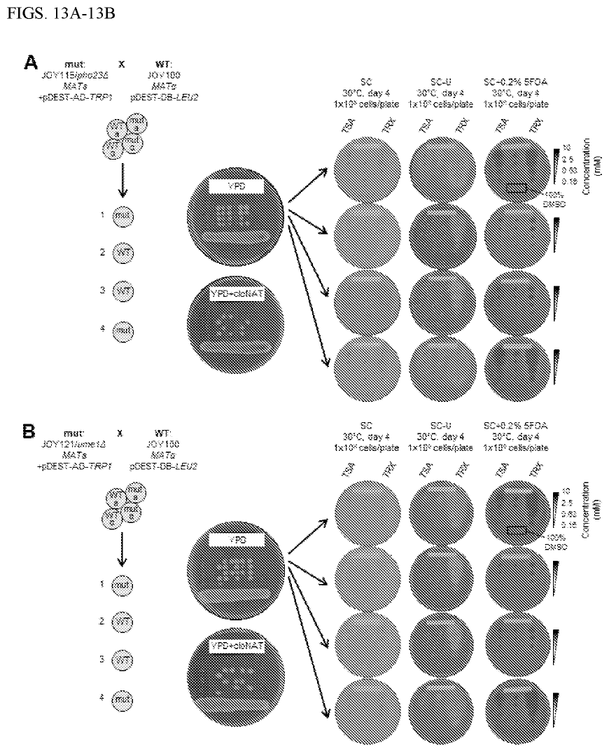

Finally, to identify potential chemical-genetic interactions between deletions of Rpd3L HDAC subunits and compounds affecting the function of the complex to use them as classification tools, every knockout was systematically tested against traditional HDAC enzymatic inhibitors. FIG. 11 shows that, compared to wild-type conditions, enhanced Ura⁺/Foa⁻ phenotypes are obtained for TSA and TRX, in a dose-dependent manner, when Pho23 or Ume1 is deleted. These synergistic interactions between TSA or TRX and pho23Δ or ume1Δ were demonstrated to be both necessary, by rescuing the wild-type phenotype with plasmids expressing PH023 or UME1 (FIGS. 12A and B), and sufficient, by crossing deletion strains with wild-type cells and analyzing the resulting haploid spores from the different tetrads (FIGS. 13A and B). The effects are recapitulated in FIG. 2L, and compounds functionally affecting components of the Rpd3L HDAC complex are thus expected to also give rise to enhanced Ura⁺/Foa⁻ phenotypes, when tested in the same conditions. Altogether, results presented in FIG. 2 demonstrate the robustness of our assays to classify and verify new perturbants from high-throughput chemical screening, and compare them to well-established HDAC enzymatic inhibitors.

### Example 3: Identifying Functional HDAC Complex Perturbants in a High-Throughput Setting

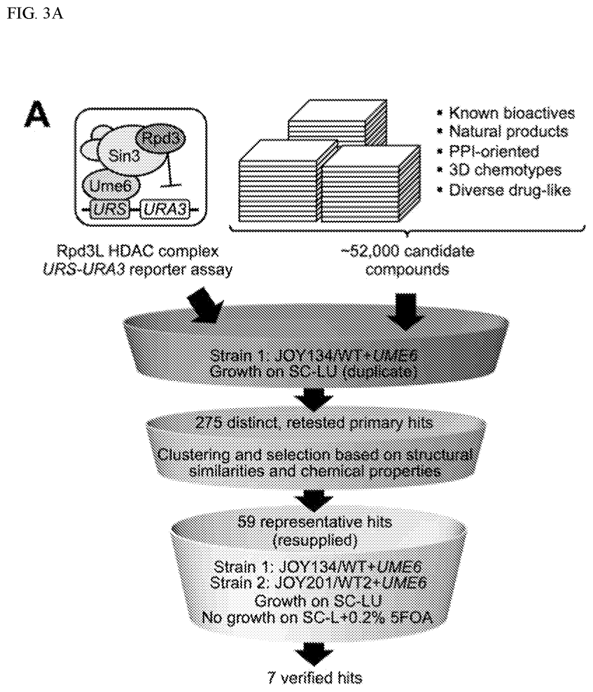

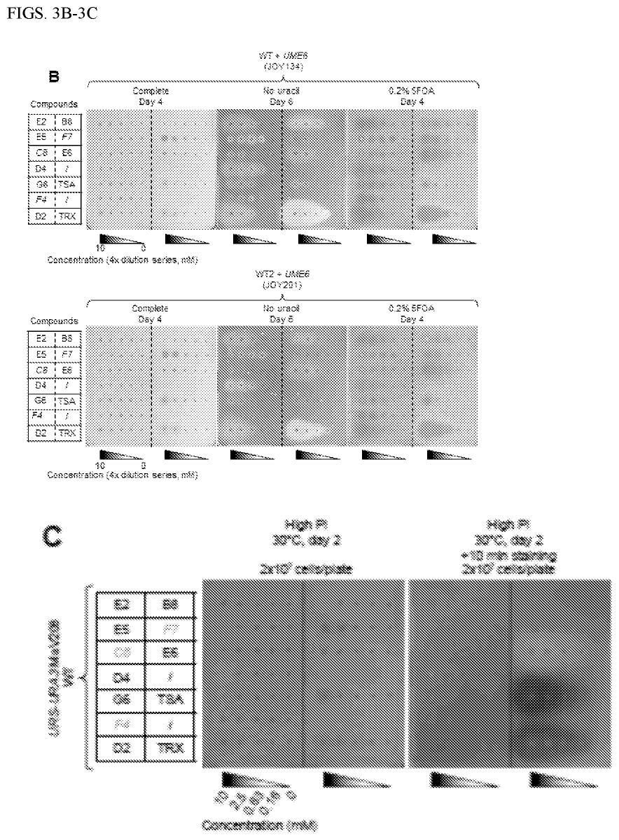

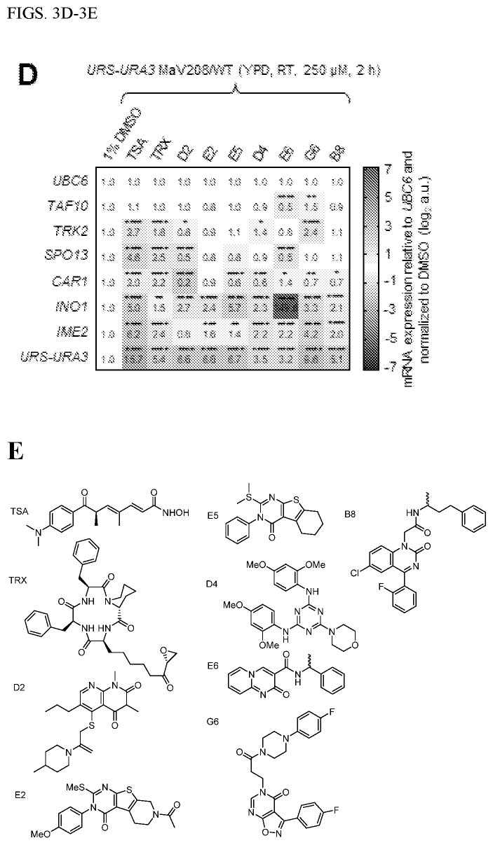

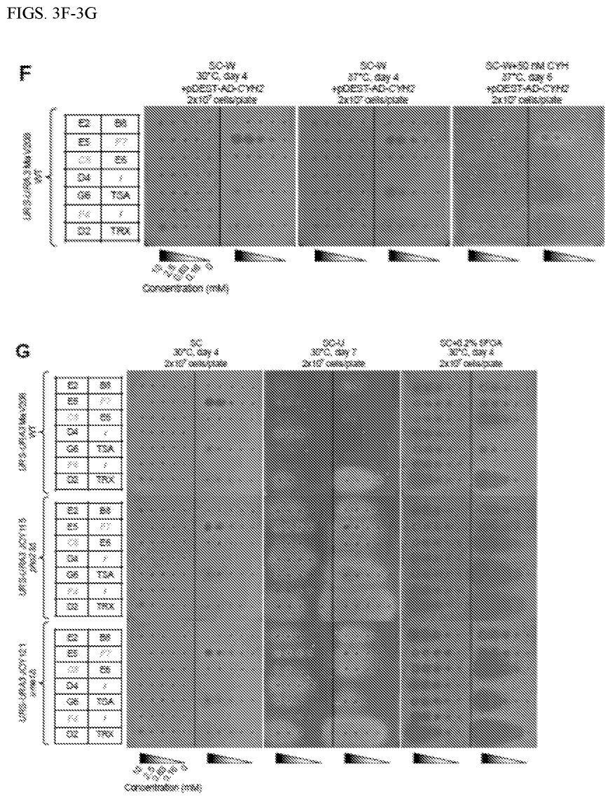

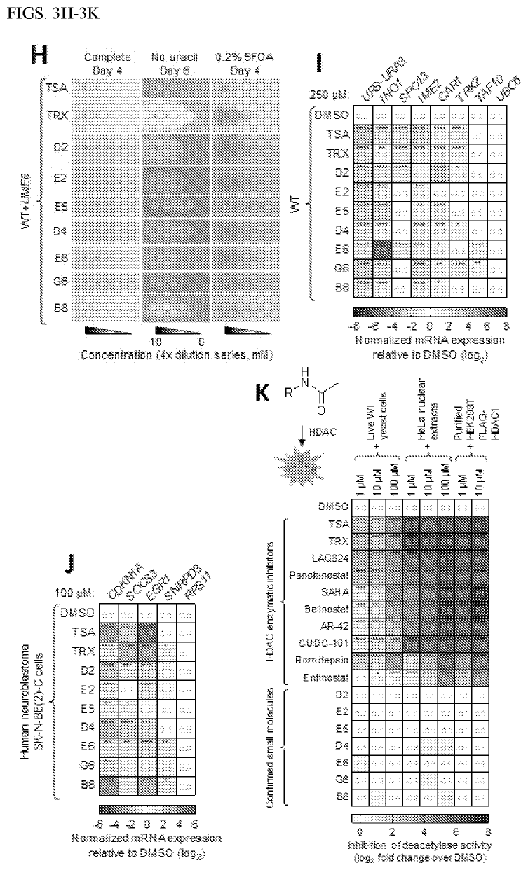

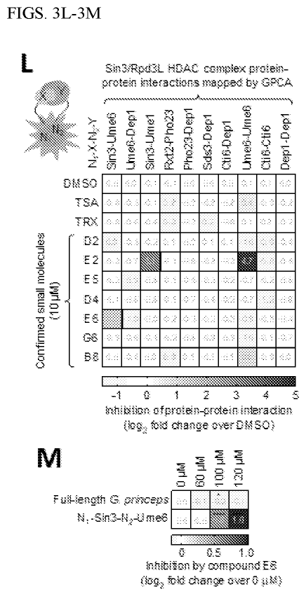

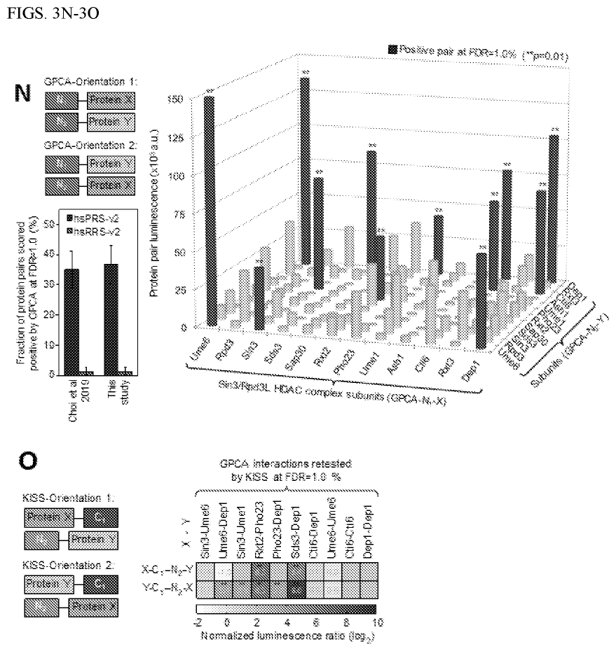

To broadly explore the different ways of perturbing the Rpd3L HDAC complex, ˜52,000 candidate compounds interrogated were with the URS-URA3 reporter assay in the JOY134 yeast strain (i.e. MaV208+UAE6; FIG. 29, FIG. 3A). These came from subsets of diverse, high-quality chemical libraries including known bioactives such as commercially-available drugs, natural products, molecules with PPI inhibition properties according to published studies [Hamon et al 2013], diversity-oriented synthesized compounds containing complex three-dimensional chemotypes [Brown et al 2011], and annotated molecules with drug-like qualities and potential for structure-activity relationship (SAR) follow-up experiments (FIG. 32). These libraries were chosen to give us a representative coverage of the chemical space, and increase chances of finding molecules targeting under-explored functionalities of HDAC complexes. Out of the ˜52,000 tested compounds, 275 gave rise to Ura⁺ phenotypes of varying intensities after retest, resulting in a ˜0.5% primary hit rate (FIG. 3A, FIG. 32). After clustering compounds based on their structural similarities, 59 representative hits (i.e. one to three hit(s) per cluster) were prioritized according to criteria like: potency, absence of labile/reactive chemical groups, and absence of metabolic liabilities such as functional groups that are prone to first pass metabolism by cytochrome P450 enzymes. Interestingly, none of the compounds displayed the traditional, TSA-like chemotype of HDAC enzymatic pocket binders, since no obvious “cap group/linker/zinc-binding group” scaffold was identified amongst the different structures [Li et al 2019].

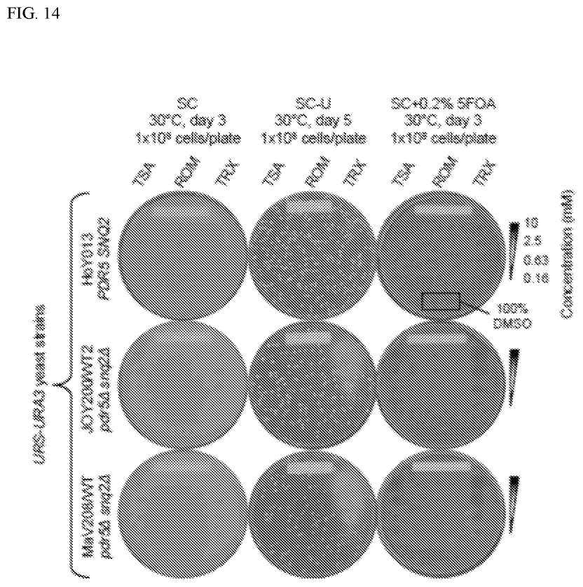

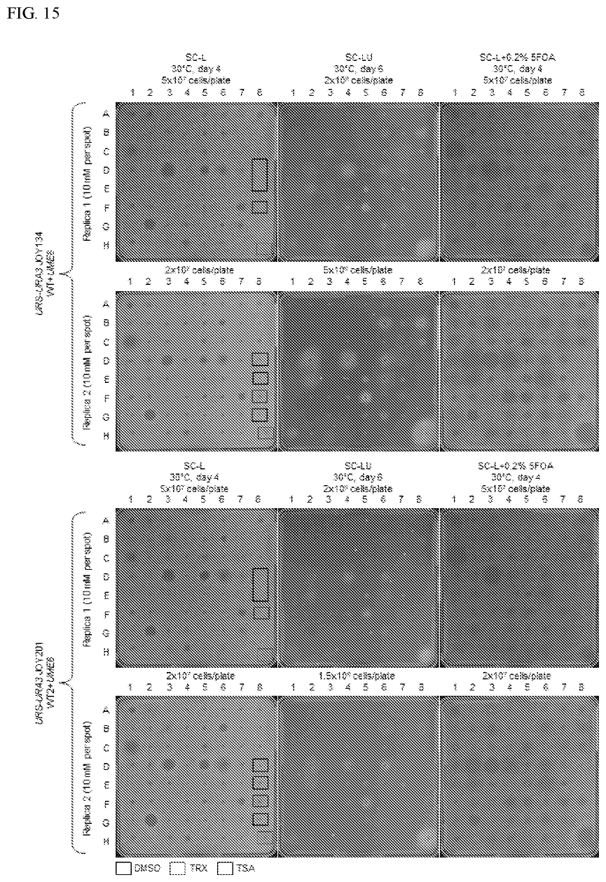

Finally, to identify the most robust hits to follow in this study, dry powders were acquired of the 59 prioritized compounds from the different suppliers, and retested them using a second wild-type yeast strain (WT2), JOY200, where the SPAL10::URA3 reporter system was reconstructed de novo from a parental Y8800-like strain, HoY013 (FIG. 10E and FIG. 15). In particular, JOY200 contains the double snq2Δ pdr5Δ deletion that increases its sensitivity to chemicals, and was generated from a parental Y8800-like strain, HoYO13 (FIG. 14). Phenotypic divergences between S. cerevisiae MaV and Y yeast cells have been described in the literature [Braun et al 2009], which suggests that the genetic backgrounds of those two strains are different. Therefore, the URS-URA3 reporter assay in JOY200 constituted a second biological replicate of the original URS-URA3 assay used for high-throughput chemical screening. Among the 59 representative hits, only compounds that consistently conferred Ura⁺, and mirror Ura⁺/Foa⁻ phenotypes in several repeats of the URS-URA3 reporter assay in both strains were picked for further studies (example of retests shown in FIG. 15). This resulted in seven verified hits for which the Ura⁺/Foa⁻ phenotypes are shown in FIG. 3B and FIG. 3H (three examples of hits not selected for further studies are also shown; grey italic). The purity of those compounds was confirmed to be >90-95% by liquid chromatography-mass-spectrometry (LC-MS). Remarkably, compared to two established HDAC enzymatic inhibitors, TSA and TRX, none of the seven hits were able to induce the Rpd3-sensitive acid phosphatase activity, and E6 even displayed an opposite behavior in the assay (FIG. 3C). This suggested that they were likely perturbing the Rpd3L HDAC complex without affecting its enzymatic component.

Interestingly, none of them have structural features characteristic of HDAC enzymatic pocket binders such as TSA or TRX (FIG. 3E). Altogether these results suggest that those small molecules might constitute a new class of SIN3 HDAC complex inhibitors that do not target the enzymatic activity of the complex To further compare the effect of the verified hits to that of TSA and TRX, they were all tested in the same assays described in FIG. 2I-L. FIG. 3D shows that the new compounds conferred much more heterogeneous effects on transcriptional regulation, measured by RT-qPCR, than TSA and TRX. Their structures, corresponding to five distinct chemotypes, are displayed in FIG. 3E. Importantly, all compounds significantly derepressed the URS-URA3 reporter gene used for the screen by ˜3 to 16-fold, which suggests they are indeed disrupting the Rpd3L HDAC complex at that locus. INO1 expression was also generally increased by ˜1.5 to 6-fold by the different treatments, with the exception of hit E6 that consistently derepressed this gene by an extra order of magnitude, i.e. ˜150-fold, compared to the DMSO baseline control. Interestingly, this appears to phenocopy the effects of ume6Δ, sin3Δ, or WT+UAE6 at INO1 (FIG. 2B). At the exception of hit D2, overall IME2 expression was elevated by ˜1.5 to 6-fold upon the different treatments. However, except for TSA and TRX, TRK2 expression was globally not affected by the compounds, which might be correlated with the strikingly different composition of the Rpd3L HDAC complex at that locus (FIG. 2B). Interestingly, only TSA and TRX consistently derepressed CAR1 and SPO13, while hit D2 decreased the basal expression level of those genes by, on average, ˜3 and ˜2-fold, respectively. Such increased repression situations are not surprising since classical HDAC enzymatic inhibitors are also known to confer these opposite effects on transcriptomes [Glaser et al 2003]. In addition, deleting subunits like Ash1 or Cti6 could also lead to increased repression states (FIG. 2B). Together, these RT-qPCR results indicate that, in addition to potentially leading to higher specificities than traditional HDAC enzymatic inhibitors, some of the new perturbants, such as D2, might also target proteins involved in transcriptional activation mechanisms. Importantly, FIG. 3G shows that all compounds gave rise to synergistic interactions, i.e. enhanced Ura⁺/Foa⁻ phenotypes, when tested in strains lacking Pho23 or Ume1, which, along with the RT-qPCR data at URS-URA3 (FIG. 3D), suggests they are disrupting the Rpd3L HDAC complex at that locus. Altogether, these results indicate that, compared to well-established enzymatic HDAC inhibitors such as TSA or TRX, the new type of perturbants identified with the URS-URA3 reporter assay appear to disrupt HDAC complexes with higher specificity.

To compare the transcriptional responses of the selected molecules to those of TSA and TRX, we measured expression of the Rpd3-regulated genes used to study the effect of subunit deletions. As expected, all compounds significantly derepressed the URS-URA3 reporter gene, with effects ranging from ˜3 to 16-fold. While TSA and TRX activated all Rpd3-regulated loci as expected, compounds from our screen showed higher selectivity and derepressed only some genes (FIG. 3I). Interestingly, while INO1 expression was increased by ˜1.5 to 6-fold by most molecules, the effect of compound E6 was an order of magnitude higher (˜150-fold) than any other treatment, including TSA and TRX. These results indicate that in contrast to established enzymatic HDAC inhibitors, these small molecule disruptors may affect only specific Sin3/Rpd3L HDAC sub-complexes.

Finally, since the Sin3/Rpd3L HDAC complex is highly conserved in eukaryotes, we tested the effects of the seven compounds on the expression of three established genes regulated by the orthologous SIN3 HDAC complex in human cells: CDKN1A, SOCS3, and EGR1. To benchmark the compounds, we tested them in human neuroblastoma SK-N-BE(2)-C cells [Frumm et al 2013, Phimmachanh etl 2020]. As expected, TSA and TRX both derepressed CDKN1A, SOCS3, and EGR1. In contrast, the seven molecules identified with the URS-URA3 reporter assay induced different derepression effects on the tested genes (FIG. 3J).

### Example 4: Determining the Mode of Action of Verified HDAC Complex Perturbants

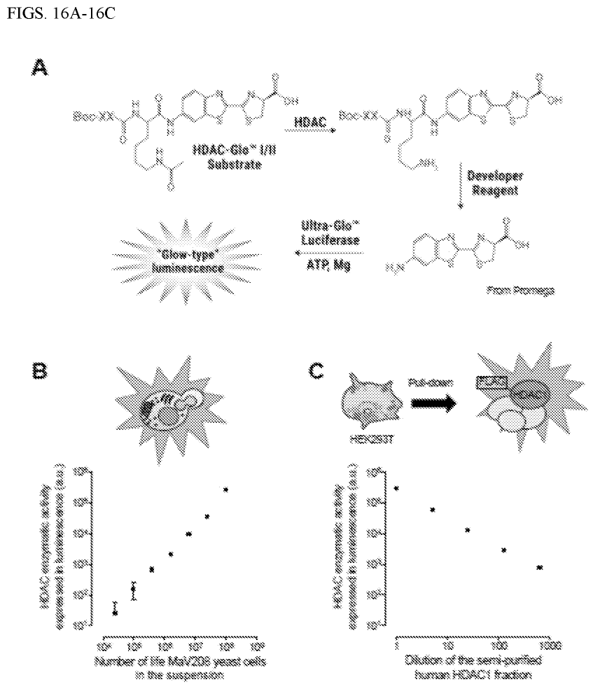

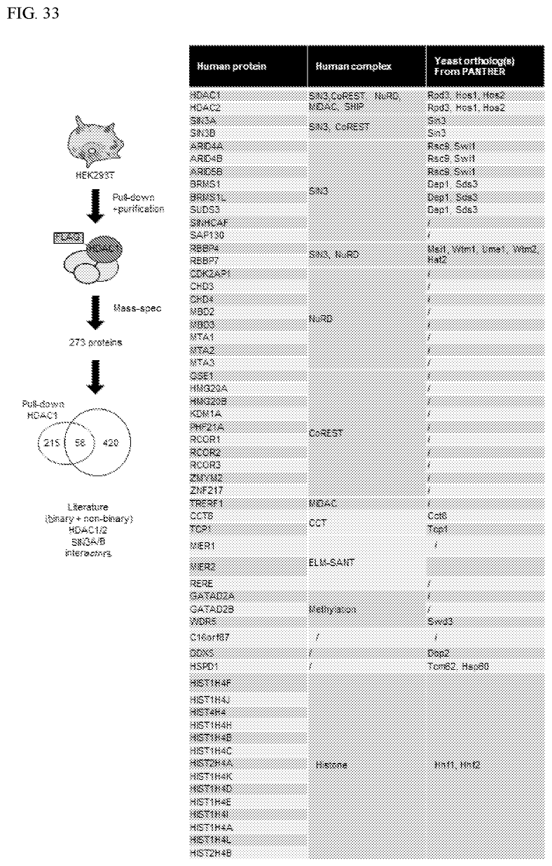

To understand how the seven verified hits perturb HDAC complexes, their ability to inhibit enzymatic activity was looked at using the well-established HDAC Glo™ I/II assay kit developed by Promega [Hsu et al 2016]. This single reagent addition, luminescent assay allows one to measure the relative activity of HDAC class I and II enzymes using live cells, cellular extracts, or purified proteins in culture plates. In particular, the assay uses a cell permeable, acetylated, luminogenic peptide substrate that can be deacetylated in the presence of functional HDAC enzymes. This HDAC-mediated deacetylation of the peptide results in its cleavage from the substrate by a protease-containing reagent, which releases aminoluciferin, and allows luminescence quantification upon addition of a recombinant firefly luciferase (FIG. 16A). In summary, the experimental setting is such that normal HDAC activity is represented by relatively high luminescence values, while enzymatic inhibitors decrease the luminescence signals. Three different sources of HDAC enzymes were used to test our compounds in this study: 1) live S. cerevisiae MaV208 yeast cells, 2) human HeLa nuclear extracts (provided in the assay kit), and 3) N-terminally FLAG-tagged human HDAC1, pull-down from HEK293T cells [Fischle et al 2002]. Importantly, as reported in the literature, and observed here in the mass-spectrometry analysis of the semi-purified FLAG-HDAC1 fraction, the human HDAC sources contain, among other complexes, the SIN3 complex (ortholog of the yeast Rpd3L HDAC complex) from which many subunits are conserved between yeast and human (FIG. 33) [Banks et al 2020]. To work under conditions of high sensitivity, and prevent signal saturation, it was first determined the linear range of the assay for the different HDAC sources (FIG. 16B-C). In addition, to control for assay specificity, 10 well characterized, positive control HDAC enzymatic inhibitors, and 19 bioactives not known to target HDAC enzymes, were tested along with the seven verified hits, at different concentrations.

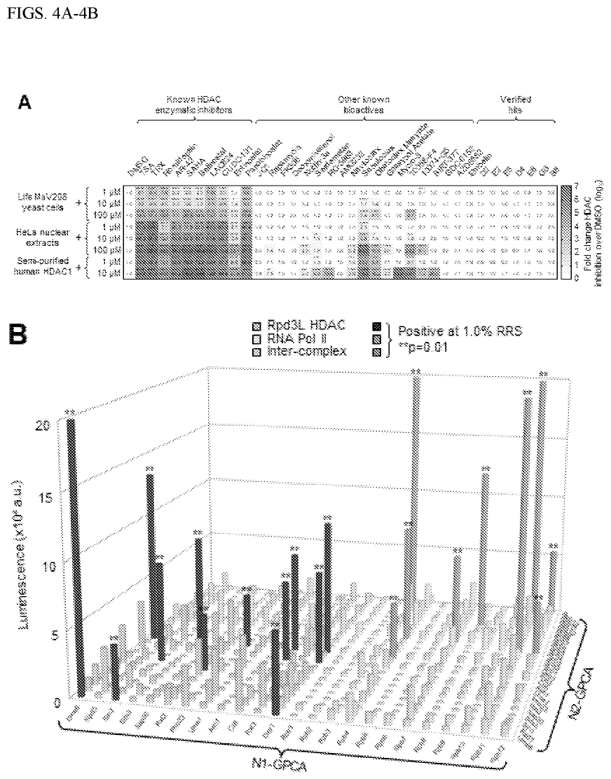

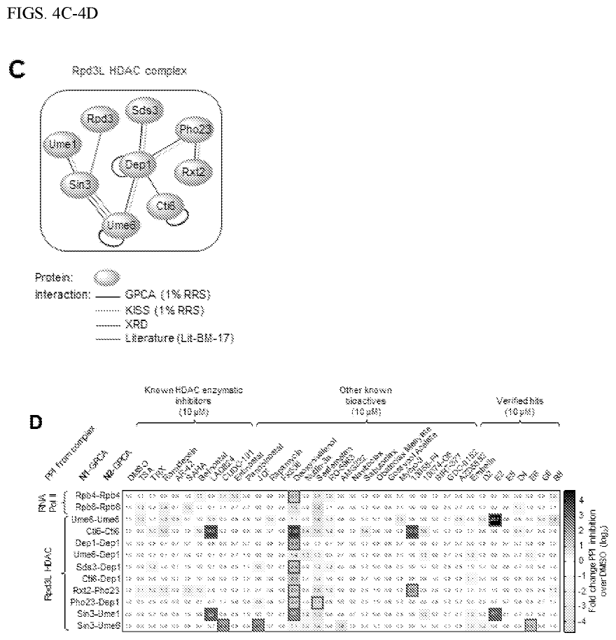

As shown in FIG. 4A, the positive controls inhibited HDAC activity of live yeast cells, HeLa nuclear extracts and semi-purified HDAC1, while most of the other bioactives left HDAC activity unperturbed, except at higher concentrations. Thus, in the different attempts presented here, none of the seven verified hits have consistently scored as HDAC enzymatic inhibitors, which is in line with their behaviors in other experiments (FIGS. 3C, D, F).

Since the targeted phenotypic URS-URA3 reporter assay allows identification of compounds with alternative disruption mechanisms, it was investigated whether the seven verified hits could dissociate binary PPIs within the Rpd3L HDAC complex. First, binary, direct interactions between the 12 subunits of the Rpd3L HDAC complex were systematically mapped using the mammalian cell-based G. princeps complementation assay (GPCA) [Cassonnet et al 2011, Choi et al 2019]. The open reading frames (ORFs) of the different full-length subunits into N1- and N2-GPCA plasmids were cloned which allowed expression of the tested proteins, X and Y, as N-terminal fusions of the two luciferase fragments, Luc1 and Luc2. A luminescence signal is then produced if Luc1-X and Luc2-Y interact. In particular, in the primary, all-by-all GPCA PPI screen implemented, every protein X is tested against every protein Y in two different orientations, since it is systematically permuted from one assay tag to the other, ultimately assessing both, Luc1-X/Luc2-Y and Luc1-Y/Luc2-X orientations (FIG. 17A). To work under conditions where the GPCA assay maximizes detection of true positive interactions while minimizing recovery of random protein pairs, performance was benchmarked, in parallel to the all-by-all screening, against two well-established, independent reference sets of human protein pairs [Braun et al 2009]: a positive reference set (hsPRS-v2), made of 60 well-documented PPIs used as positive controls, and a random reference set (hsRRS-v2), consisting of 78 protein pairs not known to interact, and serving as negative controls [Choi et al 2019, Trepte et al 2021]. Optimal screening conditions can thus be identified when a maximum of hsPRS-v2 PPIs are recovered under conditions where none, or nearly none, of the hsRRS-v2 pairs are scored positive. Here, a luminescence threshold corresponding to a recovery rate of 1.0% hsRRS-v2 pairs was set up, which allowed detecting 37% of the positive control hsPRS-v2 PPIs (p=4.01×10⁻¹⁰). Importantly, pairs identified by Choi et al under the same conditions were also all recovered here, which, in addition to demonstrate the high reproducibility of GPCA, showed a correct calibration of the assay for binary PPI mapping (FIG. 17B). As the same hsRRS-v2 detection threshold was applied to score interactions in the all-by-all GPCA screen run in parallel, it was expected, in theory, to recover ˜40% of all binary PPIs within this complex. Noticeably, this means that ˜60% of all other potential PPI targets were not tested in this study. Since the 3D arrangement of the Rpd3L HDAC complex is still unknown, it was also decided to include the 12 subunits of the structurally solved yeast S. cerevisiae RNA Pol II complex in the all-by-all PPI screening pipeline [Armache et al 2005]. This has allowed us to control for both, the quality of the mapping by looking at structurally supported, positive control PPIs, and its specificity in regards of the hsRRS-v2 threshold applied for the screen, by looking at the number of inter-complex protein pairs recovered. Indeed, as those two complexes are not known to share any of their respective subunits, inter-complex pairs from the screen were used as negative controls. Consequently, the all-by-all mapping was done on a search space of 24×24 proteins, which covered all possible pairwise combinations. As shown in FIG. 4B, a total of 21 (17 distinct) interacting protein pairs were recovered by GPCA, as they scored above the luminescence threshold of 1.0% hsRRS-v2 detection (empirical p=0.01). Importantly, interactions from the different complexes, 12 (10 distinct) for Rpd3L HDAC (dark blue) and 8 (7 distinct) for RNA Pol II (dark orange), clustered apart from each other's, while only one inter-complex pair (dark grey) was scored positive. Among the 66 possible heterodimer interactions in the RNA Pol II search space, three (out of the 23 direct contacts reported in the crystal structure) scored positive by GPCA (13%), while none of the 43 possible indirect contacts were recovered. For the Rpd3L HDAC complex, one interaction reported in the literature (restricted to binary PPI detection methods; Lit-BM-17 [Luck et al 2020, Calderone et al 2013, Grigat et al 2012, YeRI project]), and supported by a co-crystal structure (this study), Sin3-Ume6, was also detected by GPCA (FIG. 4C). Together, these results show that the primary GPCA PPI screen was implemented in high specificity conditions since it detected well-established, positive control interactions while minimizing recovery of inter-complex pairs.

To further validate the 10 Rpd3L HDAC binary PPIs detected by GPCA, they were retested them using kinase substrate sensor (KISS), a high-performance, orthogonal binary interaction assay [Lievens et al 2014, Choi et al 2019]. Every pair of proteins, X and Y, was tested in both orientations, as either, C- or N-terminal fusions of the assay tags, T1 and T2, i.e. X-T1/T2-Y versus Y-T1/T2-X (FIG. 17C). This specific version of KISS was selected since it gave the highest recovery rate for this assay [Choi et al 2019]. Using data from Choi et al, it was first calculated the normalized luminescence ratio (NLR) corresponding to 1.0% hsRRS-v2 detection. Only interacting pairs that scored above this NLR cutoff were considered as validated by KISS (empirical p=0.01). In such conditions, ˜60% of the hsPRS-v2 PPIs detected by GPCA were also recovered by KISS (FIG. 17B). As shown in FIGS. 17D and 4C, a comparable validation rate (50%) was obtained for Rpd3L HDAC interactions since, among the 10 PPIs initially mapped with GPCA, five were also retrieved by KISS. Altogether, these results suggest that the binary interaction map of the Rpd3L HDAC complex reported here should provide a robust set of potential PPI targets to interrogate against compounds.

Therefore, every interacting pair from FIG. 4C was subjected to the verified hits using GPCA. In this setting, compounds capable of penetrating cells and inhibiting PPIs decreased the luminescence signal compared to the DMSO control. Here, the classical enzymatic HDAC inhibitors and other bioactives (known to bind well-established targets; e.g. Nutlin-3a binds to MDM2) were used to control for specificity. Two interactions from the RNA Pol II complex also served as negative controls, as none of the hits were supposed to disrupt them. As observed in FIG. 4D, E6 specifically inhibited Sin3-Ume6 by ˜2.5-fold, while leaving all other PPIs unperturbed. Therefore, hit E6 specifically dissociated one of the most characterized interactions of the complex (FIG. 4C), i.e. the one occurring between the large scaffold protein, Sin3, and the DNA-binding protein, Ume6. This compound might thus potentially be used to prevent Ume6-mediated recruitment of the Rpd3L HDAC complex at specific chromosomal loci, while leaving other loci unperturbed.

While known HDAC enzymatic inhibitors, including TSA and TRX systematically reduced HDAC activity from all three sources, none of our prioritized compounds showed any effect (FIG. 3K). Together with the lack of structural features typically found in HDAC enzymatic pocket binders, these experiments strongly suggest that the compounds identified by our phenotypic screen do not act by inhibiting HDAC enzymatic activity.

To test for alternative disruption mechanisms, it was investigated whether the seven verified molecules could inhibit Sin3/Rpd3L-mediated transcriptional repression by perturbing intra-complex PPIs. First, binary interactions were systematically mapped between the 12 full-length subunits of the complex under highly specific and sensitive conditions [Choi et al 2019] using the mammalian cell-based Gaussia princeps complementation assay (GPCA) (FIG. 3N), and performed orthogonal validations using the kinase substrate sensor (KISS) assay (FIG. 30). Then we used GPCA to test the ability of TSA, TRX and each of the prioritized compounds to perturb binary interactions within the Sin3/Rpd3L HDAC complex. It was also found that only two molecules disrupt subunit-subunit interactions, including compound E6 that selectively inhibit the well-documented Sin3-Ume6 PPI (FIG. 3L). The inhibitory effect for E6 on the Sin3-Ume6 interaction was verified and we also confirmed that the compound does not perturb the full-length G. princeps luciferase used for complementation in GPCA (FIG. 3M). E6 was thus selected for further studies.

### Example 5: Confirming the Disruption of HDAC Complexes by a Protein Interaction Inhibitor

To determine if derepression of URS-URA3 by hit E6 requires a functional Rpd3L HDAC complex to be observed, the effect of this compound was tested, by RT-qPCR, in the absence of the following subunits: Rpd3, Sin3, and Ume6. UBC6 and TAF10, two genes not known to be regulated by Rpd3, were used as controls for specificity, and expression levels were calculated relative to the DMSO conditions. The enzymatic HDAC inhibitor TSA was also used as a control in those experiments.

FIG. 5A shows that the derepression effect of TSA at URS-URA3 was not measured in the absence of its target, Rpd3, or in the absence of Sin3 and Ume6, but required a wild-type complex to be observed. The same observation was made for hit E6 as derepression of URA3 was not measured in the absence of the proteins involved in its potential PPI target, Sin3 and Ume6. A functional Rpd3L HDAC complex is thus required to induce derepression of URA3 by the compounds.

To further determine if derepression of the URS-URA3 reporter gene by compound E6 was dependent on a functional Sin3/Rpd3L HDAC complex, we tested its effect in the following yeast strain backgrounds: rpd3Δ, sin3Δ, ume6Δ and sin3Δ/ume6Δ (FIG. 5J). As expected, TSA had no effect on URS-URA3 expression in these mutant strains. Similarly, the absence of these subunits drastically decreased the ability of E6 to derepress the URS-URA3 locus, suggesting that this compound is epistatic to the tested deletions and acts on a fully assembled, functional Sin3/Rpd3L HDAC complex.

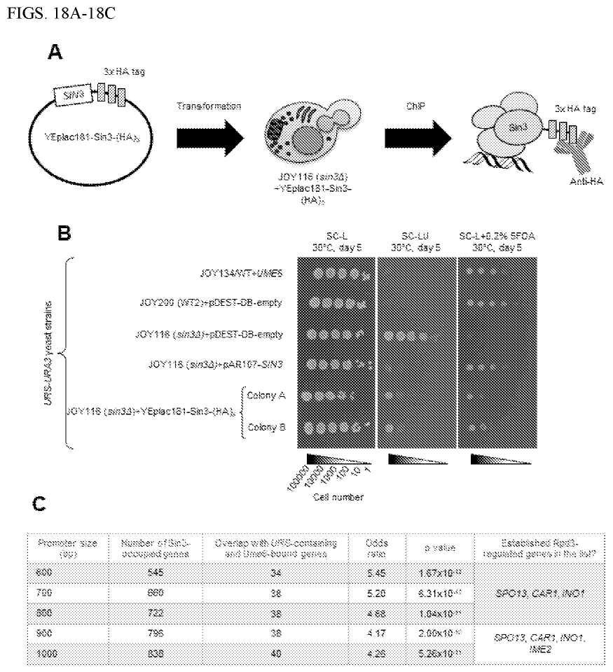

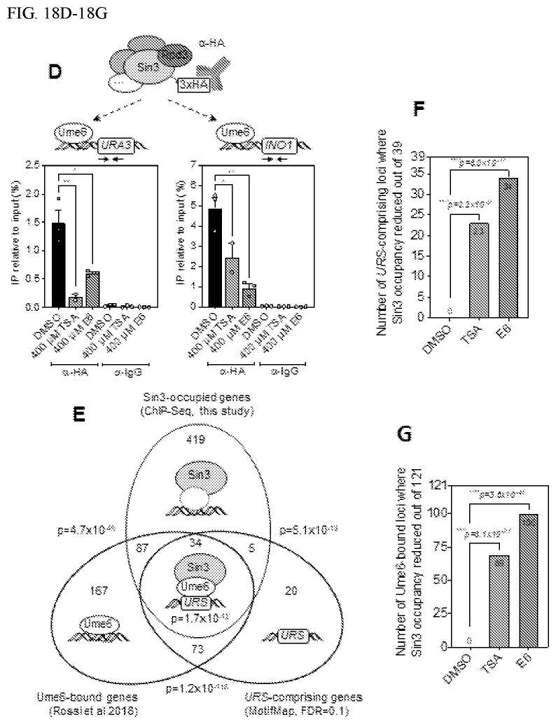

To test whether E6 disruption of the Sin3-Ume6 interaction prevents recruitment of the HDAC complex to its target loci, we performed chromatin immunoprecipitation (ChIP) experiments in a sin3Δ yeast strain expressing an HA-tagged Sin3 [Kadosh et al 1997] (FIGS. 18A-B). Using ChIP-qPCR we verified that E6 significantly decreased the presence of Sin3 at URS-URA3 and INO1 (FIG. 18D). Interestingly, TSA also appeared to prevent recruitment of Sin3, in agreement with studies suggesting that enzymatic inhibitors can lead to dynamic remodeling of HDAC complexes [Sardiu et al 2014]. To compare the effects of enzymatic (TSA) and PPI (E6) inhibitors on the recruitment of Sin3 on a genome-wide scale, we performed ChIP-sequencing (ChIP-seq) experiments. In the absence of inhibitors, promoters of 545 genes were occupied by Sin3 (FIG. 5K). Importantly, there were significant overlaps between these Sin3-occupied genes and genes containing at least one URS-binding site motif in their promoter (132 total), and genes bound by the Ume6 TF (361 total) (FIG. 18E). Treating yeast cells with TSA significantly decreased the presence of Sin3 at 295 genes, while compound E6 had a more dramatic effect with Sin3 occupancy reduced at 450 genes (FIG. 5K). Interestingly, upon systematic examination of the URS-containing and/or Ume6 target genes, we found that although E6 decreased Sin3 occupancy to a greater extent than TSA, it did not dissociate Sin3-containing complexes at every locus (FIGS. 18F and 18G and FIGS. 5L and 5M), as illustrated by ChIP-seq tracks in the surroundings of three Ume6-bound genes that also contain a URS motif in their promoter (FIG. 5N).

To confirm the mode of action of hit E6 in cellulo, it was evaluated as to how this compound affects recruitment of the Rpd3L HDAC complex at different chromosomal loci by chromatin immunoprecipitation followed by next-generation sequencing (ChIP-Seq). In order to immunoprecipitate the Sin3 scaffold protein from yeast, the JOY116 (sin3Δ) strain was first transformed with a well-documented 2p plasmid expressing Sin3, C-terminally tagged with three HA epitopes, i.e. Sin3-(HA)₃ (FIG. 18A) [Kadosh & Struhl 1997]. As shown in FIG. 18B, the Sin3-(HA)₃ construct complemented sin3Δ as it gave rise to phenotypes that are closer to wild-type than to sin3Δ cells on media lacking uracil or supplemented by 5FOA, indicating recovery of the Rpd3L HDAC complex function. The cells were treated for two hours before pulling-down Sin3-(HA)₃-containing Rpd3L HDAC complexes with a well-established, ChIP-grade anti-HA antibody [Pal et al 2018]. In addition to E6, also included was the enzymatic HDAC inhibitor, TSA. All samples received the same amount of DMSO solvent (0.6% of the total volume). Quality control ChIP-qPCR analyses were conducted, prior to sending samples for sequencing, at URS-URA3, i.e. the reporter gene used to identify compounds, and at INO1, i.e. the most responsive gene upon E6 treatment (FIG. 3D, FIG. 34). UBC6, a commonly used housekeeping gene, was also checked in those preliminary experiments. In addition, pull-downs with isotype anti-IgG served as negative controls. As observed in FIG. 5B, compared to the DMSO condition, both E6 and TSA decreased occupancy of Sin3-(HA)₃ at URS-URA3 and INO1, but not at UBC6 as expected, since this gene is not known to be occupied by Sin3. These observations confirmed the initial PPI inhibition effect observed with E6 by GPCA (FIG. 4D). Interestingly, TSA also seemed to disrupt recruitment of the complex at URS-URA3 and INO1, which is consistent with other studies suggesting that enzymatic HDAC inhibitors can lead to a dynamic remodeling of the networks and have differential effects on protein interactions [Sardiu et al 2014]. The exact mechanism on how enzymatic inhibitors can induce such perturbations is still unclear, but one could hypothesize that they might result from indirect, allosteric changes when the compound binds to the catalytic HDAC pocket.

To further generalize those ChIP-qPCR observations, and see if differences between enzymatic and PPI inhibitors could be found at a genome scale, the same samples were analyzed, in duplicate, by next generation sequencing. A total of 545 genes were found to be occupied by Sin3 in both replicates, when using a promoter size cutoff of 600 bp (FIG. 18C). In order to check the quality of this result, the list of Sin3-occupied genes were compared to genes containing at least one URS-binding site motif in their promoter (132 total) [Daily et al 2011], as well as genes bound by Ume6 (361 total) according to published ChIP-Seq/ChiP-exo results [Rossi et al 2018]. In all cases, significant overlaps (p<˜1×10⁻¹²) between conditions were observed (FIG. 5C), and 34 genes were shared by all three conditions (p=1.7×10⁻¹²), i.e. URS-containing, Ume6-bound, and Sin3-occupied. This analysis suggested that the list of Sin3-occupied genes obtained by pulling-down Sin3-(HA)₃ via an anti-HA antibody was of good quality.

FIG. 5D shows that treating yeast cells with either 400 μM E6, or TSA significantly decreased Sin3 occupancy at 360 (i.e. 545-185), or 142 (i.e. 545-403) genes, respectively. The difference between those two compounds likely correlates with their modes of action: direct PPI inhibition for E6 versus enzymatic inhibition for TSA, potentially followed by allosteric changes in the complex. Indeed, the fact that E6 decreases Sin3 occupancy for a larger fraction of genes compared to TSA indicates that this compound might indeed prevent recruitment of the Rpd3L HDAC complex at particular loci, by directly inhibiting the Sin3-Ume6 interaction. In addition, as shown in FIG. 5E, the overlap of genes where Sin3 occupancy was not affected by E6 (i.e. 121 out of 185 total, p=7.6×10⁻⁸⁹) was also significant, which showed that this compound does not perturb recruitment of the Rpd3L HDAC complex at every chromosomal locus where Sin3 is present.

To evaluate the extent to which E6 or TSA could decrease Sin3 occupancy in the promoter of genes that also contain an URS motif, or are bound by the Ume6 TF, compared the outputs of these two treatments were compared to the same gene lists presented in FIG. 5C. FIGS. 5F-G shows a similar trend as the one observed at genome scale for the two compounds (FIGS. 5D-E). Indeed, E6 also decreased occupancy of Sin3 at more genes compared to the enzymatic inhibitor, TSA, in both cases, which was also the case for the intersection of URS-containing and Ume6-bound genes (107 total). These effects appeared to be specific as comparing results to 107 genes randomly picked among the yeast S. cerevisiae's reference genome (excluding the 107 candidates from the intersection of URS-containing and Ume6-bound genes) did not lead to the same enrichments.

In order to zoom in on specific locations in the yeast genome, the integrative genomics viewer (IGV) software [Robinson et al 2011] was used. In particular, the ChIP-Seq traces of the well-established Rpd3-regulated genes used in FIGS. 2B and 3D were looked at, as well as couple genes containing a URS motif in their promoter, and bound by the Ume6 TF. As shown in FIG. 5H for both replicates, Sin3 occupancy (DMSO condition; black traces) was reduced upon E6 treatment (red traces), at INO1, IME2, and CAR1, but not at SPO3 or TRK2. Interestingly, compared to the enzymatic HDAC inhibitor, TSA (grey traces), the PPI inhibitor, E6, had a much stronger effect on Sin3 occupancy at INOL. This observation might explain why E6 derepresses INO1 by an extra order of magnitude compared to TSA (FIG. 3D).

Finally, to evaluate the impact of E6 on the recruitment of HDAC complexes at genes bound by other Sin3-interacting TFs, TF-binding motifs within the MotifMap database were first systematically curated [Daily et al 2011], and compared the resulting list of TFs to that of Sin3-occupied genes. As shown in FIG. 5I, a total of 16 Sin3-interacting TFs were identified that, altogether, bind to 840 genes. Remarkably, except for genes bound by Hsf1, E6 decreased Sin3 occupancy in all other groups of TF-bound genes, to different extents. For example, E6 decreased Sin3 occupancy at all Opi1-bound genes, while it only disrupted half of the Tod6-bound genes. Altogether, these findings strongly suggest that hit E6 prevents recruitment of the Rpd3L HDAC complex at specific chromosomal loci by inhibiting particular interactions between the scaffold protein Sin3, and TFs such as Ume6.

### Example 6: Biophysical Validations of the HDAC Complex Protein Interaction Inhibitor

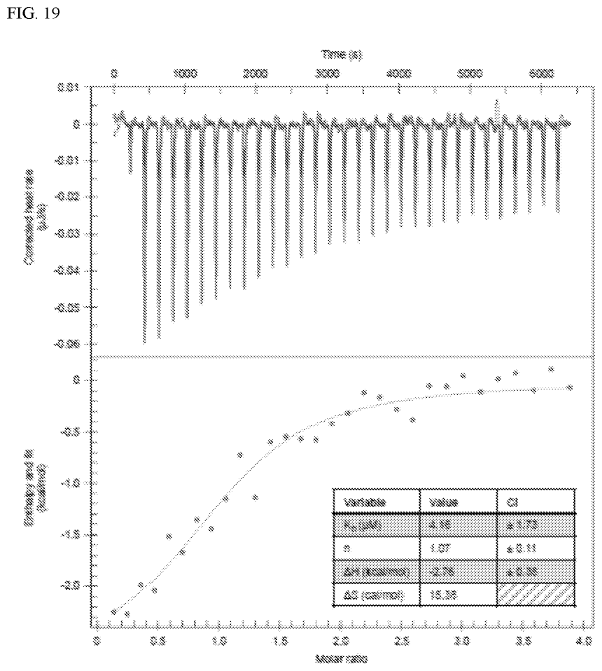

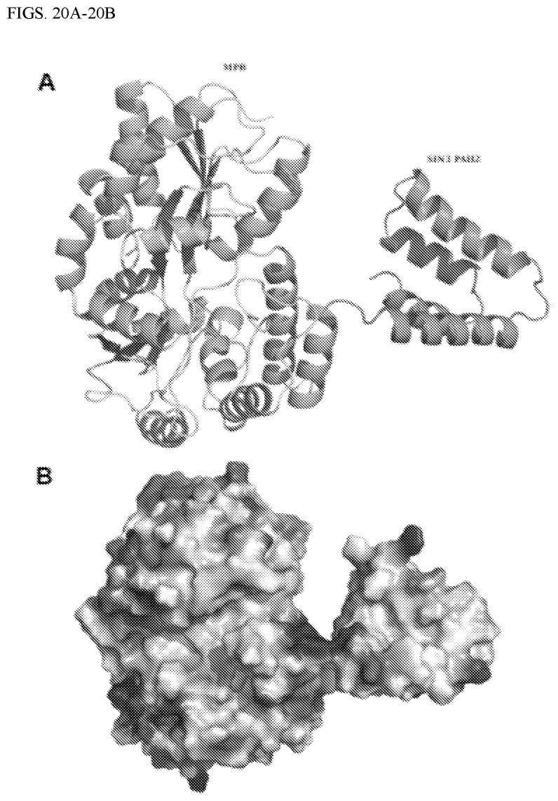

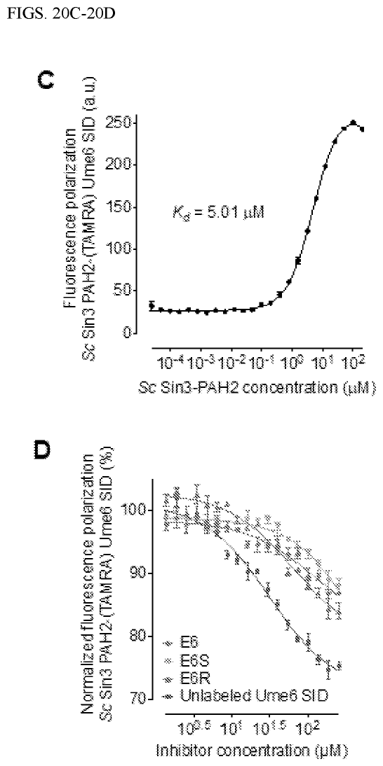

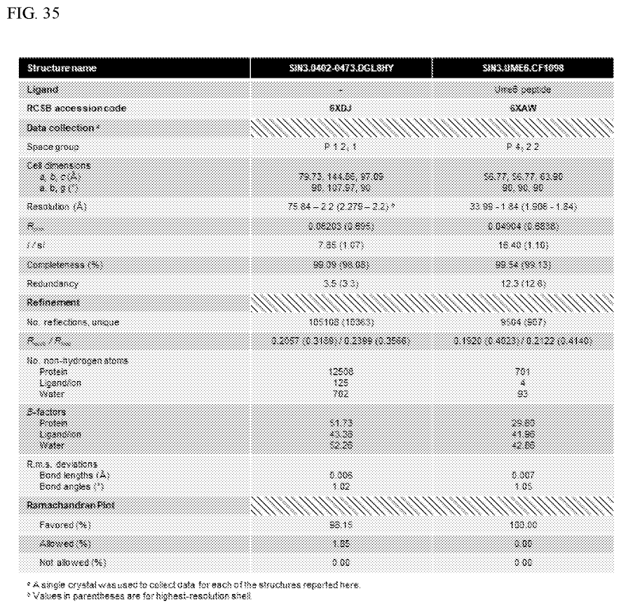

To characterize how hit E6 perturbs the binary Sin3-Ume6 PPI target, this interaction was reconstituted out-of-cells to prevent any potential interference that might come from other cellular components. Using a fluorescence polarization (FP) assay, the interaction between the two minimal domains of Sin3 and Ume6 reported in the literature was measured [Washburn & Esposito 2001, Le Guezennec et al 2006]: the paired amphipathic helix 2 (PAH2; residues 402-473) and the Sin3-interacting domain (SID; residues 516-531), respectively (FIG. 6A). In this FP setting, Sin3 PAH2 is N-terminally fused to a glutathione S-transferase (GST) tag while Ume6 SID is labeled with a fluorescein isothiocyanate (FITC) probe at the N-terminal position. As shown in FIG. 6B, when increasing amounts of GST-Sin3 PAH2 were added to a fixed concentration (30 nM) of FITC-Ume6 SID, a plateau representing full occupation of GST-Sin3 PAH2 sites by available FITC-Ume6 SID peptides was reached. The binding affinity of this interaction was evaluated at 0.9 μM, as reflected by the calculated equilibrium dissociation constant, K_{D}. Importantly, a comparable K_{D} of 4.2±1.7 μM was measured by isothermal titration calorimetry (ITC) for the same Sin3 PAH2/Ume6 SID domain-domain interaction (FIG. 19). Following these results, it was determined the Sin3 PAH2/Ume6 SID co-crystal structure (FIGS. 6C-D, FIG. 35) as well as the apo Sin3 PAH2 domain (FIGS. 20A-B, FIG. 35) using X-ray diffraction. Similar to published solution structures of mammalian SIN3A PAH2 or SIN3B PAH2 [Brubaker et al 2000, van Ingen et al 2006], the PAH2 domain of yeast S. cerevisiae Sin3 adopts a left-handed, anti-parallel, four-helix bundle structure, two of which (helices α1 and α2) define a deep hydrophobic pocket that accommodates the Ume6 SID peptide (FIG. 6C). The hydrophobic face of the amphipathic Ume6 α helix peptide interacts with PAH2, filling the binding pocket. With a root-mean-square deviation (RMSD) between backbone atoms of 1.7 Å², the apo and complexed structures did not reveal significant conformational changes.

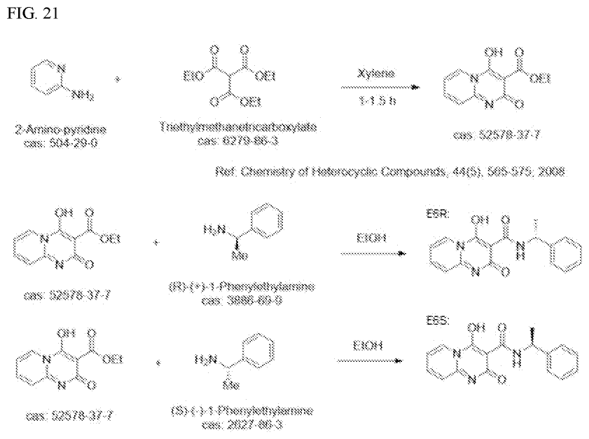

To determine if E6 could displace the Ume6 SID peptide from the Sin3 PAH2 domain, the effect of this compound by FP was tes, against the Sin3 PAH2/Ume6 SID interaction (FIG. 6B). As hit E6 is a 4-hydroxy-pyridopyrimidinone-based racemic mixture, both enantiomers, E6R and E6S were first synthesized (FIGS. 21 and 6E). As shown in FIG. 6F, while the DMSO negative control did not disrupt the Sin3 PAH2/Ume6 SID interaction, the unlabeled Ume6 SID positive control was able to compete with the FITC labeled peptide and inhibit the interaction in a dose-dependent manner, with an IC₅₀ of 57.4±2.5 μM. Comparable to this result, E6 (racemic mixture) and isolated enantiomers (E6R and E6S) were also able to displace the FITC-Ume6 SID peptide, and inhibit the Sin3 PAH2/Ume6 SID interaction, with IC₅₀ of 146.1±15.8 M, 155.1±20.3 μM, and 157.6±39.8 μM for E6R, E6S, and E6, respectively. These IC₅₀ corresponded to an inhibitory concentration (Kᵢ) of ˜30 μM, assuming that the mode of inhibition is competitive [Cer et al 2009]. Interestingly, when testing both enantiomers in the phenotypic URS-URA3 reporter assay (FIG. 6G), E6R appeared to be more potent than E6S, which was confirmed by measuring expression of URA3 upon treatment with the different molecules at different concentrations (FIG. 6H).

To test the binding of E6R to the PAH2 domain of yeast Sin3, three ligand-detected nuclear magnetic resonance (NMR) methods were used: differential line broadening (DLB), saturation transfer difference (STD), and Carr-Purcell-Meiboom-Gill-transverse relaxation rate (CPMG-R₂). First, FIG. 6I shows that, compared to the NMR spectrum of E6R alone, significant line broadening and shifting were observed when Sin3 PAH2 was present, which suggested a bound state for E6R. Second, on-resonance saturation of Sin3 PAH2 decreased the intensity of E6R peaks in the STD experiment, indicating transmission of magnetization energy from Sin3 PAH2 to proximal E6R ligands (FIG. 6J). Finally, compared to free small molecules that have slow transverse relaxation rates in CPMG-R₂ experiments, ligands engaging the target protein relax faster. Thus, a decreased relaxation time in the presence of a protein directly indicates binding of the ligand to that protein. As shown in FIGS. 6K and 6L, this situation was clearly observed for E6R in the presence of Sin3 PAH2. Together, these results strongly establish E6R as a Sin3 PAH2 binder. Importantly, as binding was confirmed in every one of these three independent NMR experiments, the likelihood of E6R being a false positive hit is drastically reduced.

To determine the binding mode of E6R, it was virtually docked against the PAH2 domain of yeast Sin3, restricted to the Ume6 peptide-binding region. FIG. 6M shows a robust complementarity for the obtained docking pose as the surface shape of the docked compound coats the surface shape of the target protein domain. For example, the benzyl group of E6R fits into the hydrophobic pocket of the Sin3 PAH2 domain (FIG. 6N). In addition, similar to how the Ume6 SID peptide binds the Sin3 PAH2 domain, the type of interactions of E6R with the protein is predominantly of hydrophobic nature (FIG. 6O), with predicted distances between interacting moieties below 4 Å.

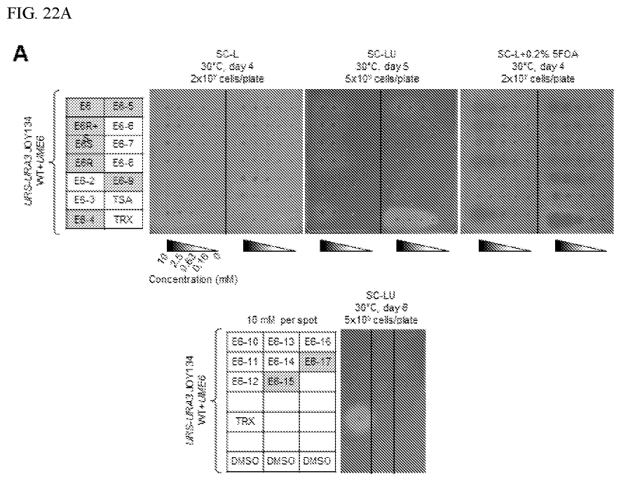

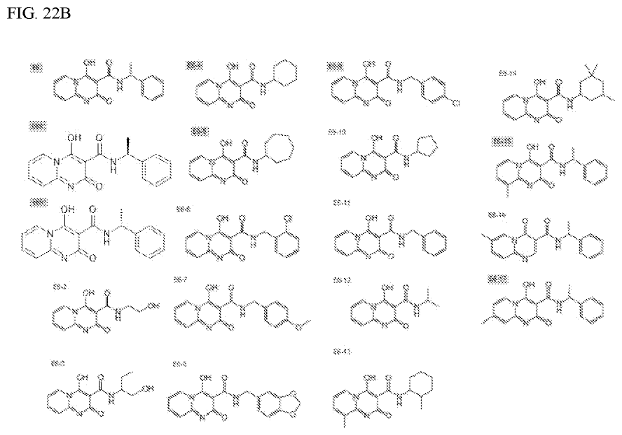

To further explore the chemical space around the scaffold of E6R, a small SAR study was performed with the goal of providing trends that could be used to design more potent analogs in future studies, and support the virtually determined compound-binding mode. In addition to supporting that E6-like compounds are acting through a specific binding interaction, several SAR lessons were also learned from testing 16 analogs in the URS-URA3 reporter assay (FIGS. 22A-B), which included that the hydroxy and methyl groups are both required for activity, as is the benzyl moiety since it cannot be readily replaced by aliphatic groups. These observations support the docking pose obtained for E6R into the PAH2 domain of yeast Sin3 (FIGS. 6M-0). Altogether, these results indicate that E6R directly inhibits the binary Sin3 PAH2/Ume6 SID interaction, by binding to the PAH2 domain of Sin3 and displacing Ume6, which is in good correlation with findings from other experiments.

### Example 6A: E6R Enantiomer Binds to SIN3 PAH2 and Inhibits the PAH2-SID Interaction

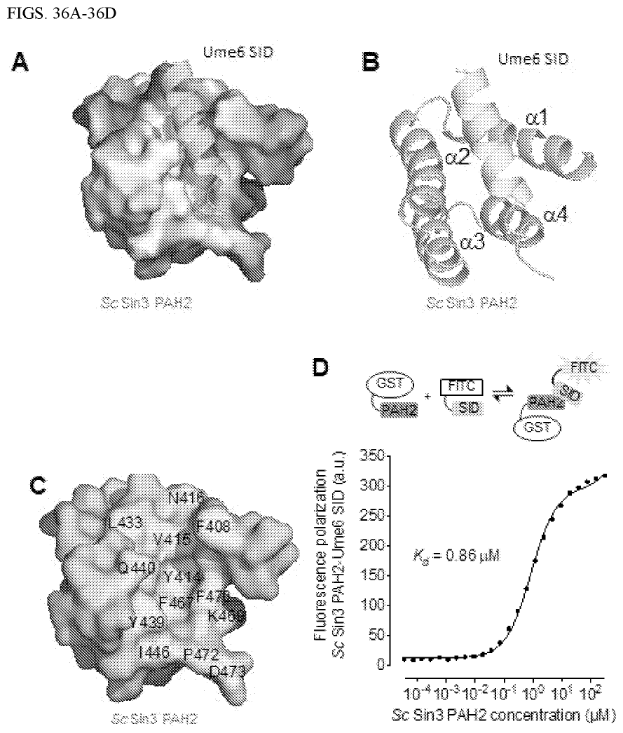

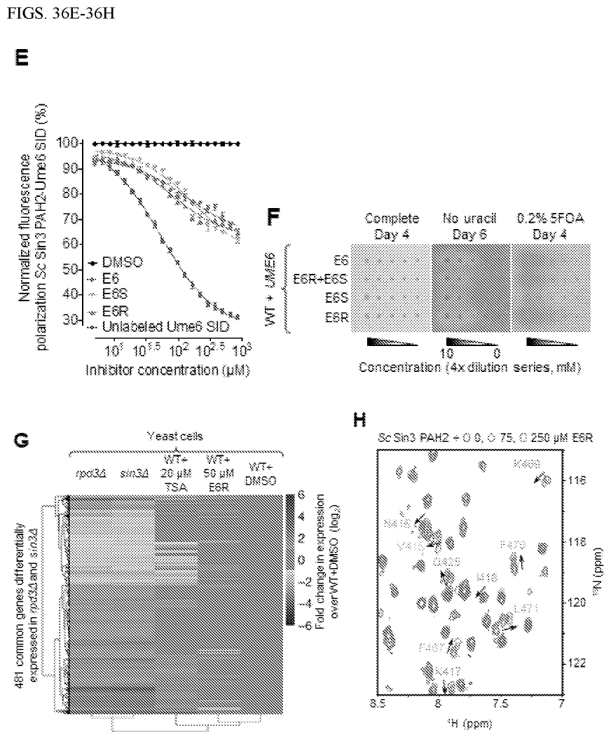

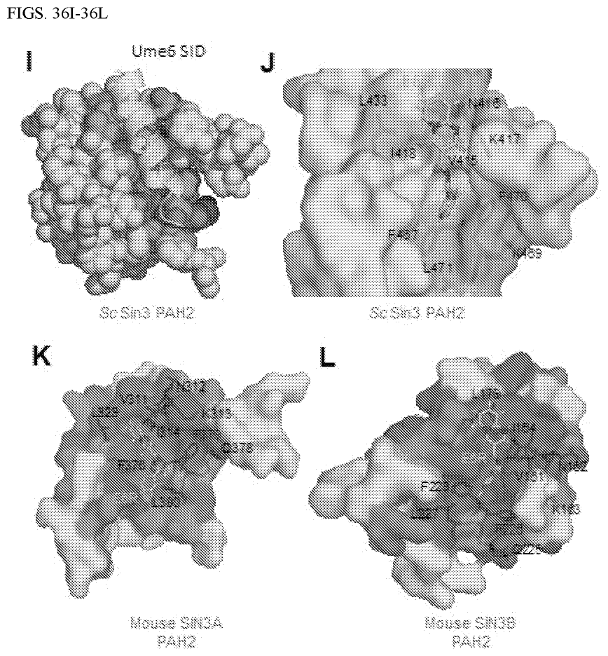

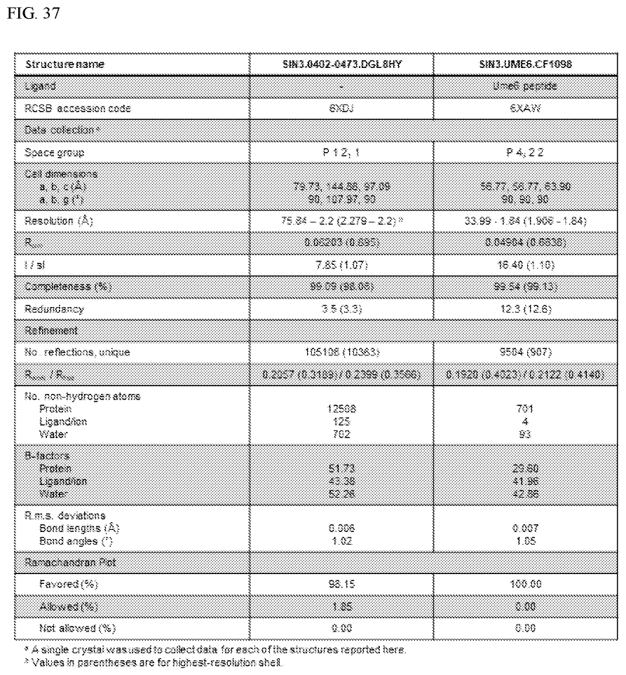

The yeast Sin3-Ume6 PPI is mediated by the paired amphipathic helix 2 (PAH2) of Sin3 and the Sin3-interacting domain (SID) of Ume6 (Washburn et al. 2001). Both domains are highly conserved in mammalian cells and allow similar assemblies between transcription factors and the SIN3A/B scaffold proteins. A K_{D} of 4.16±1.73 μM was measured by isothermal titration calorimetry (ITC) for the yeast Sin3 PAH2-Ume6 SID pair (FIG. 19) before determining the crystal structures of the yeast Sin3 PAH2 domain alone (FIG. 20A and FIG. 37), or in complex with the Ume6 SID peptide (FIGS. 36A-C and FIG. 37) at 2.2 and 1.8 Å resolutions, respectively. Similar to published solution structures of mammalian SIN3A PAH2 [Kumar et al 2011] and SIN3B PAH2 [van Ingen et al 2006], the PAH2 domain of yeast S. cerevisiae (Sc) Sin3 adopts a left-handed, anti-parallel, four-helix bundle structure, two of which (helices α1 and α2) defining a deep hydrophobic pocket that accommodates the amphipathic Ume6 α helix peptide (FIGS. 36A-C). With a root-mean-square deviation (RMSD) between backbone atoms of 1.7 Å², the apo and complexed structures did not reveal significant conformational changes.

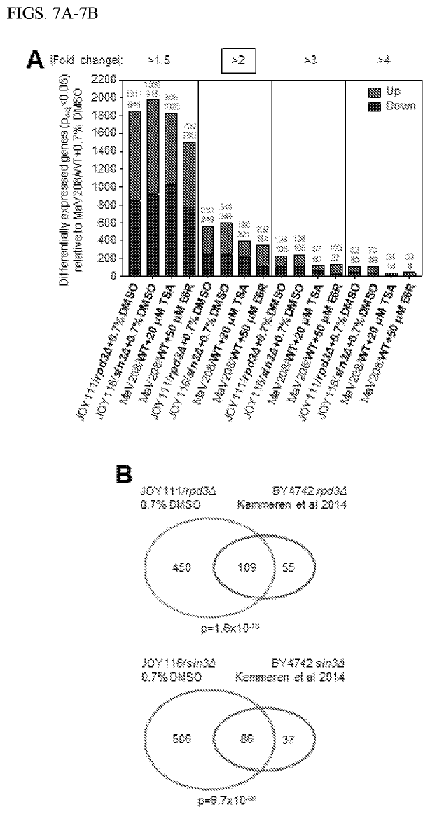

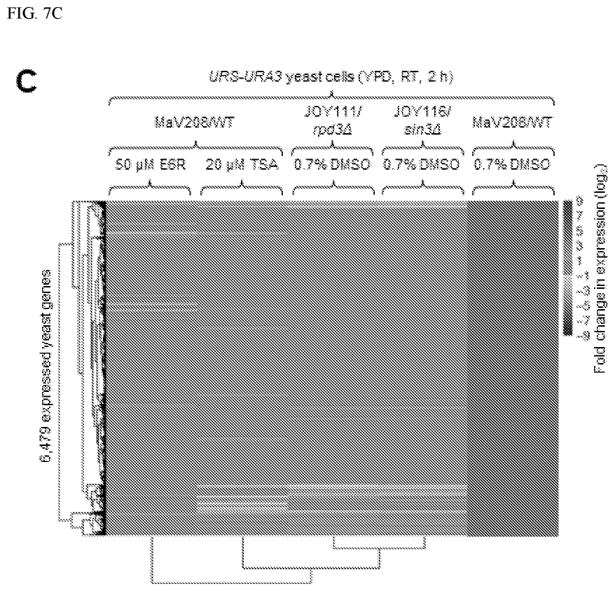

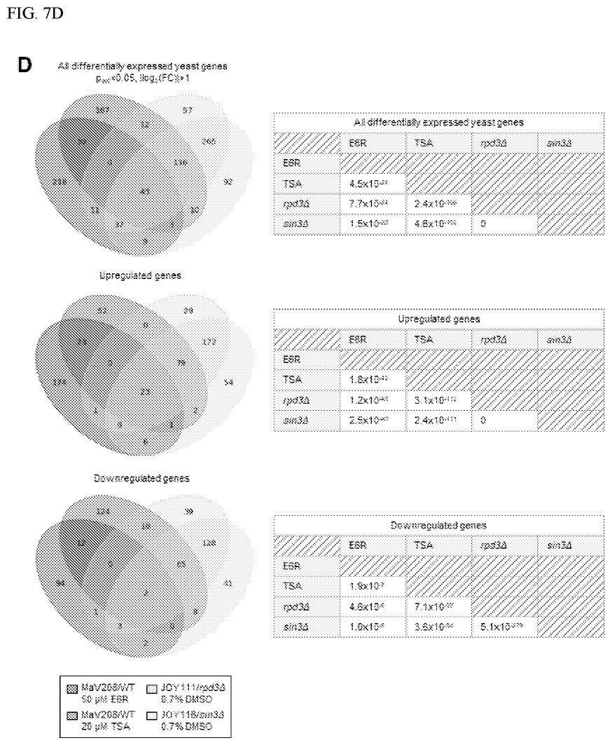

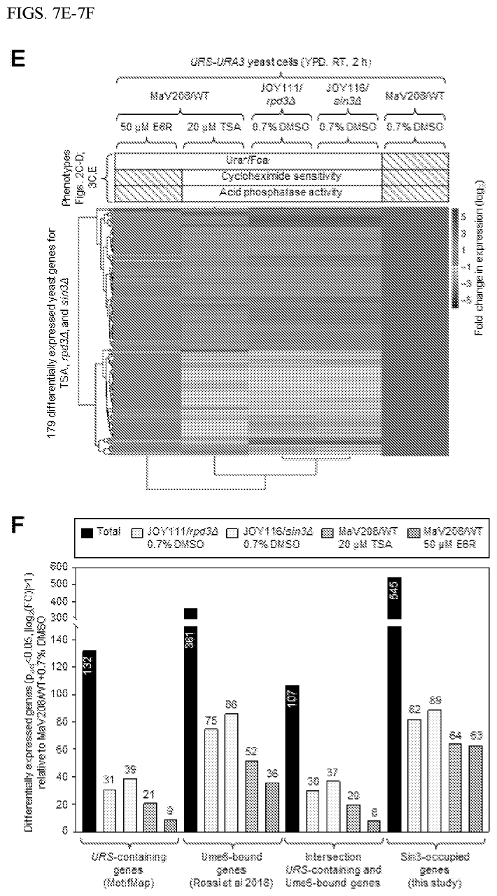

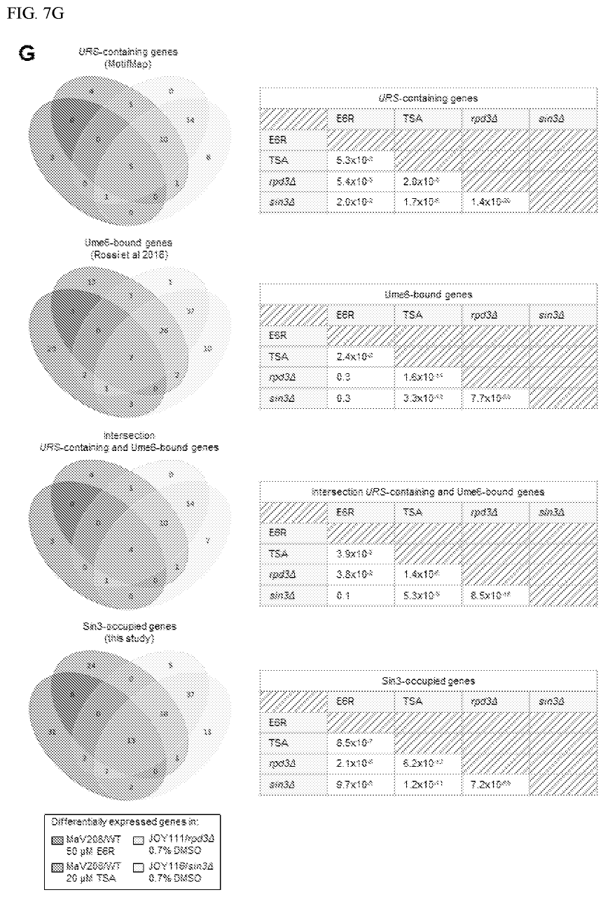

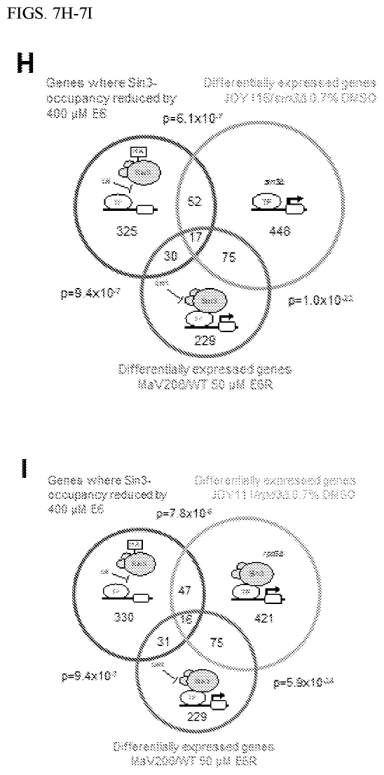

To examine inhibition of the Sin3-Ume6 interaction by E6 outside of potential interference from other cellular components, an in vitro fluorescence polarization (FP) assay was used. By adding increasing amounts of GST-Sin3 PAH2 to a fixed concentration of FITC-Ume6 SID, we estimated the interaction K_{D} as 0.86±0.02 μM (FIG. 36D). The FP was then used to test the effect of E6 on the Sin3 PAH2-Ume6 SID interaction (FIG. 36E). As E6 is a racemic mixture, the R and S enantiomers were prepared. The unlabeled Ume6 SID positive control inhibited the interaction in a dose-dependent manner, with an IC₅₀ of 53.3±2.4 μM. The E6 racemic mixture was also able to displace the FITC-Ume6 SID peptide from Sin3 PAH2 with an estimated IC₅₀ of 132.5±19.8 M, calculated as previously reported [Nishikawa et al 2016]. The E6R enantiomer was more potent than E6S, with estimated IC₅₀ values of 74.5±7.2 μM versus 174.5±21.9 μM, respectively. With an assumption of competitive inhibition, these IC₅₀ values correspond to inhibitory concentrations (Kᵢ) of 11.4, 29.1, 38.5 and 16.1 μM for unlabeled Ume6 SID, E6, E6S and E6R, respectively. Similar differences between compounds were observed when labeling the Ume6 SID peptide with TAMRA, a different fluorescent probe (FIGS. 20C-D). Taken together that the overall pattern was reproducible between FP assays using FITC or TAMRA tagged peptides, we concluded that E6R indeed competed for the Ume6 SID binding site on the Sin3 PAH2 domain. These findings were consistent with the URS-URA3 phenotypic assay as, compared to E6S, the E6R enantiomer conferred: 1) stronger rings of growth on media lacking uracil; 2) stronger growth inhibition on media containing 5FOA; and 3) stronger induction of expression at URS-URA3 (FIG. 36F and FIG. 7J). Based on these observations, E6R was prioritized for follow up studies and its transcriptional effects were compared to that of TSA on the subset of Sin3/Rpd3L HDAC complex-regulated genes in yeast following compound titrations (FIG. 7J). Interestingly, while RNA-sequencing (RNA-seq) analyses revealed that TSA alters expression of 45% of the Sin3- and Rpd3-dependent genes, E6R affected expression of 20% of this subset indicating a greater selectivity towards Sin3/Rpd3L HDAC complex-regulated genes for E6R (FIG. 36G and FIGS. 7A, 7D, and 7K).

To test the binding of E6R to the PAH2 domain of yeast Sin3, three ligand-detected nuclear magnetic resonance (NMR) methods were used: differential line broadening (DLB), saturation transfer difference (STD), and Carr-Purcell-Meiboom-Gill-transverse relaxation rate (CPMG-R₂). First, compared to the NMR spectrum of free E6R, significant line broadening and chemical shift perturbations were observed in the presence of Sin3 PAH2, indicating a bound state for E6R (FIG. 6I). Second, on-resonance saturation of Sin3 PAH2 decreased the intensity of E6R signals in the STD experiment, indicating transmission of magnetization from Sin3 PAH2 to interacting E6R ligands (FIG. 6J). Finally, the presence of Sin3 PAH2 enhanced the relaxation rate of the E6R signal in the CPMG-R₂ experiments, also indicative of E6R binding to the receptor protein (FIGS. 6K-L).

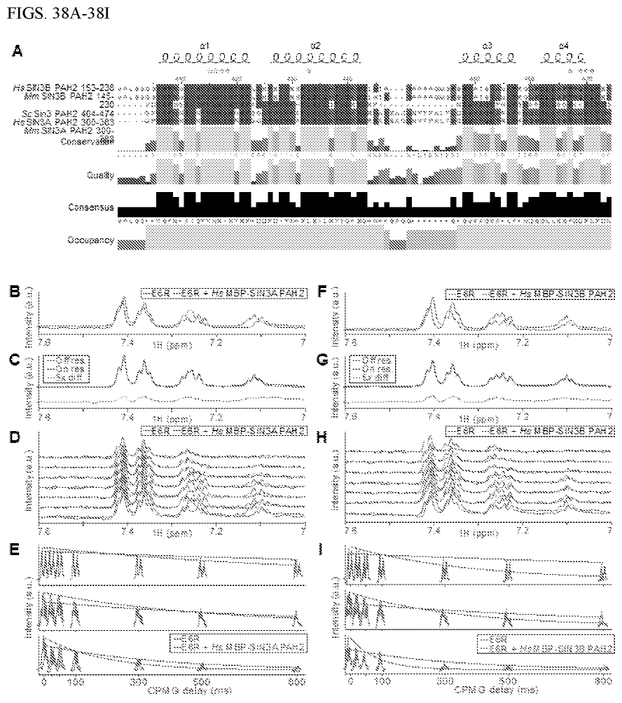

To confirm the specificity of this binding, we studied perturbations of the Sin3 PAH2 domain at the amino acid level following addition of E6R. After assigning the majority (85%) of amino acids using ¹³C- and ¹⁵N-labeled yeast Sin3 PAH2 alone by NMR (FIGS. 6P-Q), we confirmed that the secondary structures of the Sin3 PAH2 domain in solution were consistent with the crystal structure (FIG. 20A). The binding of E6R to Sin3 PAH2 was localized to only a few amino acids belonging to a subset of residues lining the Ume6 SID binding pocket, including Val415, Phe467, Phe470 and Leu471 (FIGS. 6R-S and FIG. 36H), which are conserved in mouse and human (FIG. 38A). To model the binding of E6R, we virtually docked the compound against the region of the yeast Sin3 PAH2 domain that binds the Ume6 SID peptide (FIG. 36I). Similar to the binding of Ume6 SID to the Sin3 PAH2 domain and in agreement with the NMR results (FIG. 36H), E6R is predominantly engaged in hydrophobic interactions with the Sin3 PAH2 domain (FIG. 36J), with predicted distances between interacting moieties below 4 Å. Altogether, these results establish E6R as a Sin3 PAH2 binder capable of competing with the Ume6-SID peptide.

Finally, using ligand-detected NMR, it was confirmed that E6R could bind to the conserved PAH2 domains (FIG. 38A) of human SIN3A (FIGS. 38B-E) and SIN3B (FIGS. 38F-I). The binding of E6R to the mammalian SIN3A/B PAH2 domains was then modeled by docking the compound against all NMR conformations published for those proteins [Kumar et al 2011, van Ingen et al 2006]. This revealed similar binding poses compared to the yeast model since, in both cases, E6R was predicted to bind to the same hydrophobic pocket, in the same orientation and by involving conserved amino acid positions (FIG. 38A and FIGS. 36J-L). These highly conserved binding poses in different species increase confidence in our binding model of E6R and indicate that it could potentially disrupt mammalian SIN3 HDAC complexes and associated gene regulatory functions (FIG. 3J) by binding to the PAH2 domains of SIN3A/B and inhibiting interactions in which those domains are engaged.

### Example 7. Global Effect of the HDAC Complex Protein Interaction Inhibitor on Gene Expression

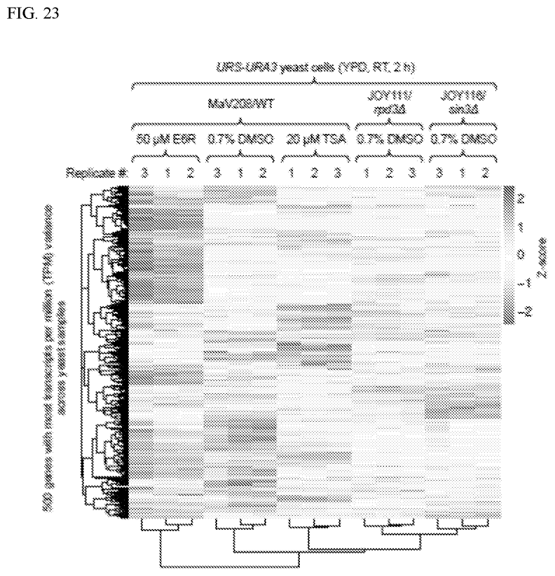

To measure the effect of E6R on the yeast S. cerevisiae MaV208 transcriptome, RNA from cells treated with this compound was sequenced, and compared results to that of cells lacking the Rpd3 or Sin3 subunit, as well as wild-type cells treated or not with TSA. All samples received the same amount of DMSO (0.7% of the total volume) for direct, side-by-side comparisons. Based on the titration experiment presented in FIG. 6H, 20 μM and 50 μM were selected to conduct RNA-Seq with TSA and E6R, respectively. Five treatments were performed for every tested condition, among which the three samples giving the closest RT-qPCR results in the pilot experiments were selected for RNA-Seq. As shown in FIG. 23, the three replicates of every condition all clustered together, demonstrating a high reproducibility between the different treatments. After testing increasing thresholds of fold change (FC) in expression for the different conditions, it was decided to focus on genes for which expression was altered by at least two fold compared to the WT+DMSO control (FIG. 7A). Using that cutoff, a total of 561 (313 up-, and 248 downregulated), 595 (346 up, and 249 down), 401 (180 up, and 221 down), or 351 (237 up, and 114 down) differentially expressed genes (p adjusted <0.05) were obtained for rpd3Δ, sin3Δ, WT+20 μM TSA, or WT+50 μM E6R, respectively. Interestingly, compared to TSA, treatment with E6R led to more up-than downregulated genes.

To benchmark our RNA-Seq results against published gene expression profiles acquired through “ChIP on chip” experiments [Kemmeren et al 2014], the differentially expressed genes in rpd3Δ or sin3Δ yeast cells were compared from both studies. Noticeably, the MaV208-based yeast strains used here have a different genetic background compared to the BY4742-based strains used by Kemmeren et al. As shown in FIG. 7B, significant overlaps between differentially expressed genes in distinct rpd3Δ or sin3Δ yeast strains were obtained when comparing the two studies under the same conditions (i.e. p<0.05, |FC in expression|>2), which strongly suggests that the gene expression profiles acquired by RNA-Seq in this work are of good quality. As highlighted in FIG. 7C, the rpd3Δ and sin3Δ conditions similarly perturbed the yeast transcriptome, and clustered together, while the WT+20 μM TSA, and WT+50 μM E6R conditions clustered separately, consistent with the different modes of HDAC complex perturbations by these two compounds: inhibiting enzymatic activity for TSA or disrupting a PPI for E6R. The corresponding overlaps between all differentially expressed genes, and genes that are up- or downregulated are shown in FIG. 7D for the different conditions. Importantly, they were all significant, which suggests these treatments likely perturb similar biological processes.

To see if a correlation between the Rdp3-sensitive phenotypes from FIGS. 2C-D and 3B,C,F, and the transcription profiles from FIG. 7C could be established; genes for which expression is altered by 20 μM TSA, rpd3Δ and sin3Δ, were looked at. Indeed, these three conditions all conferred robust Ura⁺/Foa⁻ phenotypes, sensitivity to CYH, and acid phosphatase activity, suggesting they are likely perturbing the same group of genes. However, E6 specifically gave rise to a single Ura⁺/Foa⁻ phenotype (FIGS. 3B, C, and F), and should thus affect fewer genes within this group; an assumption that likely holds for the R enantiomer of E6, E6R (FIG. 6G). As shown in FIG. 7E, out of the 179 yeast genes whose expression was changed by rpd3Δ, sin3Δ and TSA, only 43 (˜25%) were also significantly affected by E6R (p=2.07×10⁻¹⁷), including URS-URA3, which is consistent with the phenotypic profile of this compound. This result also shows E6R is targeting a subset of the Rpd3L HDAC complexes perturbed by TSA, rpd3Δ and sin3Δ.

To confirm this trend, the fractions of genes whose expression is changed by E6R, TSA, rpd3Δ or sin3Δ were compared to the same URS-containing, Ume6-bound, or Sin3-occupied genes used in FIG. 5C. As shown in FIG. 7F, E6R perturbed expression of fewer of the URS-containing and Ume6-bound genes compared to the other conditions, which is consistent with observations from FIG. 7E. However, the numbers of Sin3-occupied genes whose expression was altered in the different conditions remained comparable, with significant overlaps between them (FIG. 7G).

Finally, to measure the fraction of genes that are differentially expressed due to an E6-mediated reduction of Sin3 occupancy in their promoter, RNA-Seq and ChIP-Seq results were compared. As highlighted in FIG. 7H, significant overlaps were observed between the different tested conditions, with ˜13% (i.e. 47/351) of the genes being differentially regulated due to a decrease of Sin3 occupancy in their promoter. In addition, ˜12% (i.e. 69/592) of the differentially regulated genes in sin3Δ were similarly affected by E6. Comparable observations were made for the differentially regulated genes in rpd3Δ (FIG. 7I), which is not surprising since sin3Δ and rpd3Δ have highly similar gene expression profiles (FIG. 7C).

Altogether, results presented in FIGS. 3-7 indicate that the R enantiomer of the verified hit E6 (racemic mix), functionally disrupts the yeast Rpd3L HDAC complex at specific chromosomal loci, by binding the PAH2 domain of the scaffold protein Sin3, and directly inhibiting binary Sin3-TF PPIs, such as the well-characterized Sin3-Ume6 interaction. Interestingly, this PPI inhibitor appears to be more specific than enzymatic inhibitors such as TSA, since, among the different phenotypic assays tested here, it only gives rise to a single Ura⁺/Foa⁻ phenotype.

### Example 8. Inhibiting Specific HDAC Complexes in Mammalian Cells

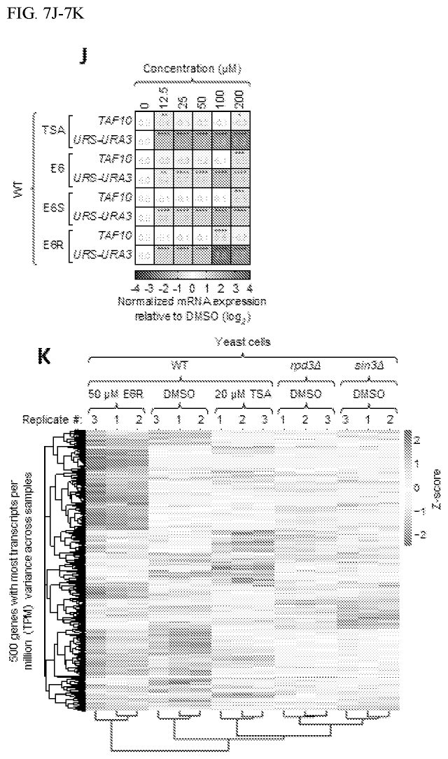

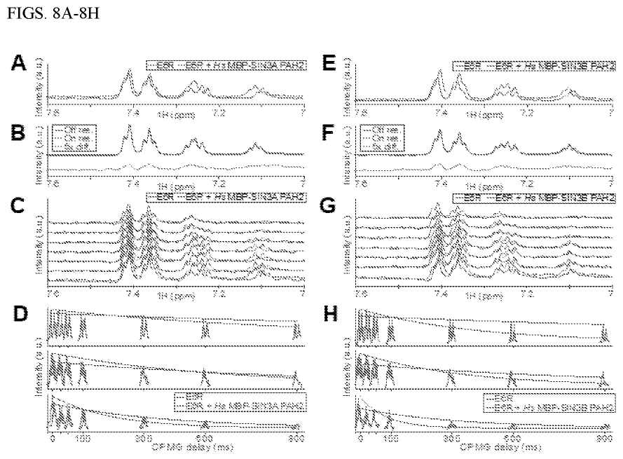

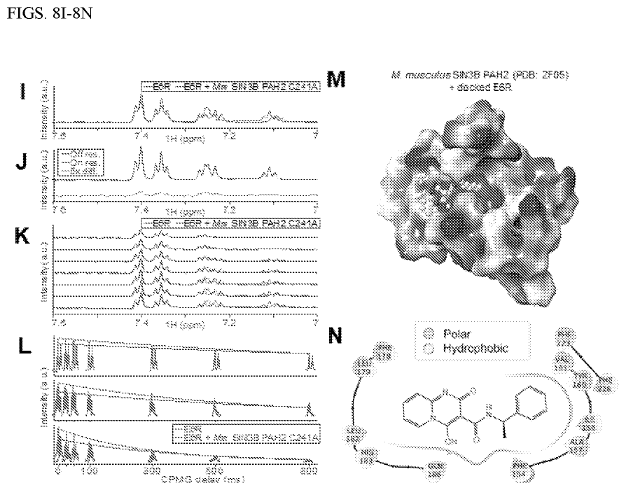

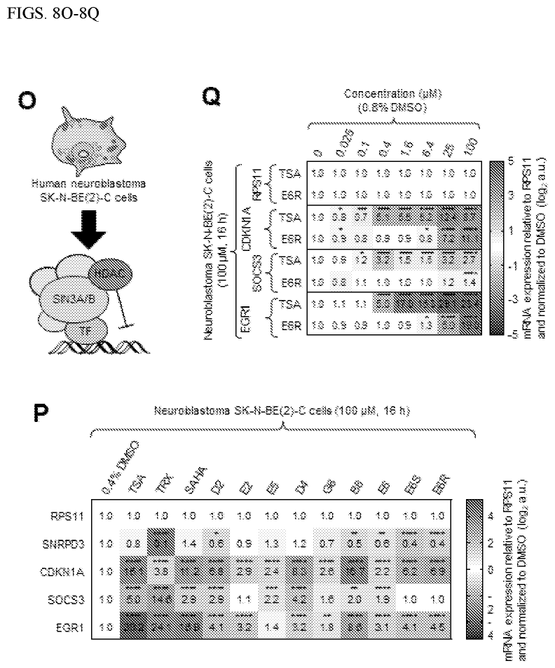

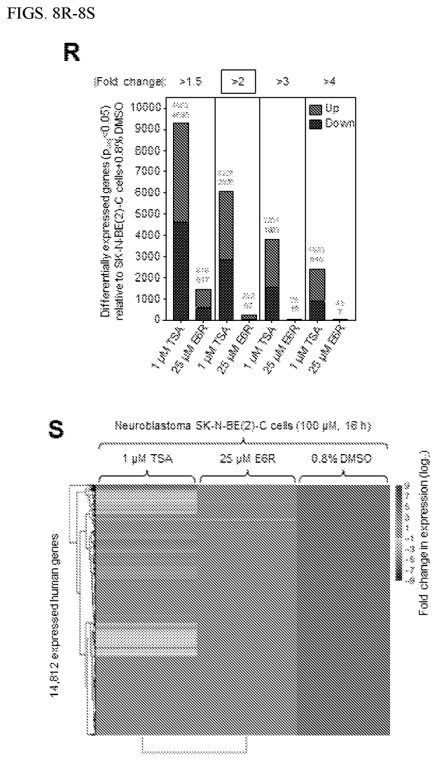

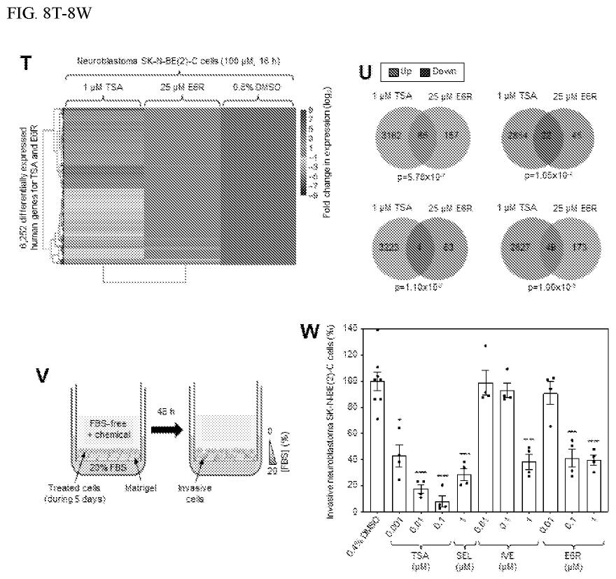

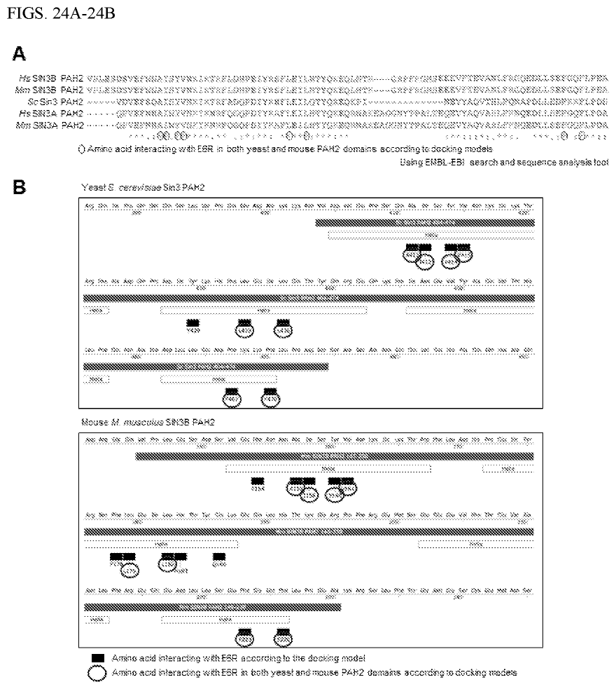

Since yeast Sin3 has two orthologs in mammalian cells, SIN3A and SIN3B, and the PAH2 domains of these three proteins are highly conserved all the way to their 3D structures (FIG. 24A), it was tested if E6R could bind the PAH2 domains of human SIN3A (FIGS. 8A-D) and SIN3B (FIGS. 8E-H), as well as the PAH2 domain of mouse M. musculus SIN3B (FIGS. 8I-L), by protein NMR. As displayed in FIGS. 8A-L, the binding results were virtually identical to those of yeast Sin3 PAH2 (FIGS. 6I-L). Interestingly, after docking E6R against all 30 NMR conformations published for the M. musculus apo SIN3B PAH2 domain [van Ingen et al 2006], an identical binding pose as for the yeast model (FIG. 6M) was identified. Indeed, the compound is predicted to bind the same pocket, in the same orientation and by involving conserved amino acid positions (FIGS. 8M-N and 24A-B). As for yeast, interactions are mostly hydrophobic but one hydrogen bond is also predicted to form between the hydroxy moiety of E6R and G1n186 of SIN3B PAH2 (FIG. 8N). The fact that the same E6R binding pose was unbiasedly obtained for PAH2 domains from two different species increases confidence in the virtually determined models and strongly suggests E6R might experimentally bind accordingly. These results also indicated that E6R could potentially disrupt mammalian HDAC complexes by binding the PAH2 domains of SIN3A and SIN3B, and inhibiting interactions in which those domains are engaged.

To assess whether E6R could affect transcription of known HDAC target genes in mammalian cells, expression changes were measured, by RT-qPCR, after treating SK-N-BE(2)-C neuroblastoma cells with this compound, along with three established HDAC enzymatic inhibitors: TSA, TRX, and SAHA. Indeed, HDAC enzymatic inhibitors have shown promising effects in this disease cell line [Frumm et al 2013], which makes it a robust model to benchmark new HDAC complex perturbants (FIG. 8O). In addition, to get an idea of the general translatability of these findings from yeast to human cells, also included were the six other verified hits from the screen, as well as E6 and E6S. Following the different treatments (100 μM for 16 h), expression levels of three genes directly regulated by HDAC complexes, CDKN1A [Ji et al 2014], SOCS3 [Icardi et al 2012], and EGR1 [Frumm et al 2013], were compared relative to RPS11, a commonly used housekeeping gene [Zhan et al 2014]. SNRPD3, a gene not know to be regulated by HDAC complexes, was employed as control for specificity (FIG. 8P). As expected, TSA, TRX, and SAHA derepressed CDKN1A, SOCS3, and EGR1. The verified hits, originally identified in yeast, all induced different derepression effects on the tested human genes. In particular, E6R and E6S, only perturbed expression of two of the tested downstream genes. Altogether, these results demonstrate that E6R not only binds to the PAH2 domains of mammalian SIN3A and SIN3B (FIGS. 8A-L), but also specifically perturbs expression of known HDAC target genes in human cells.

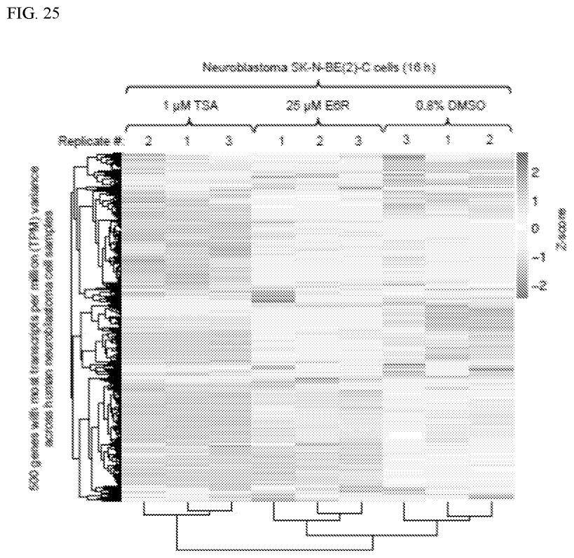

To further evaluate if E6R could globally be more specific than enzymatic HDAC inhibitors such as TSA, the transcriptomic perturbations induced by these compounds were measured in human SK-N-BE(2)-C neuroblastoma cells. Based on the titration experiment presented in FIG. 8Q, 1 μM and 25 μM were selected to conduct RNA-Seq with TSA and E6R, respectively. As for yeast data, each treatment was tested in triplicate with replicates clustering together for every individual condition (FIG. 25). After testing increasing thresholds of fold change (FC) in expression for the different conditions, it was decided to focus on genes for which expression was altered by at least two fold compared to the DMSO control, similar to the yeast analyses (FIG. 8R). FIG. 8R-S shows that E6R affected the transcriptome much more specifically than TSA since it perturbed an order of magnitude fewer genes compared to the HDAC enzymatic inhibitor: 289 versus 6,103, respectively. This trend was observed for all tested cutoffs of fold changes in expression (FIG. 8R). In addition, significant overlaps were observed between genes differentially expressed upon TSA or E6R treatments, which suggests E6R is targeting a subset of the HDAC complexes perturbed by TSA (FIGS. 8T-U).

In order to check if the specificity of E6R could lead to similar biological effects as well-established HDAC enzymatic inhibitors like TSA, or known SIN3A PAH2 binders such as ivermectin or selamectin, capabilities of these compounds to inhibit human SK-N-BE(2)-C neuroblastoma cell invasion were tested the. Indeed, recent studies have repositioned ivermectin and selamectin, two FDA-approved avermectin derivatives, as mammalian SIN3A PAH2 binders with inhibition of cancer cell invasion activity [Kwon et al 2015]. Therefore, as E6R also targets the PAH2 domain of SIN3A (and SIN3B), it was hypothesized that this new compound might act similarly. A well-established cell invasion assay was used where untreated starved cells can migrate through a matrigel to find a nutrient-rich medium, which recapitulates the in vivo property of tumor cells to invade tissues (FIG. 8V). As shown in FIG. 8W, E6R did indeed phenocopy the effect of TSA, ivermectin (IVE), or selamectin (SEL), as they all reduced the migration of neuroblastoma cells by 2.5 to 10-fold. Combined with the gene expression profiles from FIGS. 8P and 8S, these findings highlight the effective specificity of E6R since this new compound leads to similar phenotypic outcomes as a classical HDAC enzymatic inhibitor, while perturbing an order of magnitude fewer genes in mammalian cells.

### Example 9. Inhibiting Specific HDAC Complexes in a Psychosis Mouse Model

To evaluate if the HDAC complex PPI inhibitor E6R could be active in mouse, a psychosis model was selected where the conditional knockout of HDAC2 (HDAC2 cKO) in the pyramidal neurons of the frontal cortex (FC) has revealed promising therapeutic avenues (FIGS. 9A-B) [Kurita et al 2012, Hanks & González-Maeso 2013, Ibi et al 2017, de la Fuente Revenga et al 2018]. Indeed, it was shown that this conditional depletion of HDAC2 leads to a protective phenotype against psychotic behaviors induced by psychedelic drugs [Nichols 2016], such as substituted amphetamine 2,5-dimethoxy-4-iodoamphetamine (DOI). This positioned HDAC2 as an important target to treat positive symptoms such as hallucinations and delusions in patients with psychiatric conditions including schizophrenia and bipolar disorder [Kurita et al 2012]. For example, chronic treatment with the enzymatic HDAC inhibitor SAHA was shown to prevent, by targeting HDAC2, the disruptive effects of psychedelic drugs [de la Fuente Revenga et al 2018]. It follows that perturbing HDAC2-containing complexes, such as the SIN3 complex, with PPI inhibitors like E6R might also lead to similar results. In particular, it was shown by protein NMR that E6R binds to the PAH2 domains of mammalian SIN3A and SIN3B proteins (FIGS. 8A-N).

To assess whether E6R may be suitable for testing the effect of HDAC inhibition via new mechanisms in mouse, the half-life (Tim) and intrinsic clearance (Clᵢₙₜ) of this compound was first measured using a standard microsomal metabolic assay where sunitinib was used as a control [Lamberto et al 2017]. In this setting, compound degradation is induced by concentrated microsomes isolated from mouse liver. In particular, these hepatic extracts contain the cytochrome P450 system that is primarily responsible for first pass metabolism in vivo. As shown in FIG. 9C, in conditions where sunitinib had a stability of ˜10 min, the resulting stability of E6R was 17 minutes, indicating potential for exposure in animals.

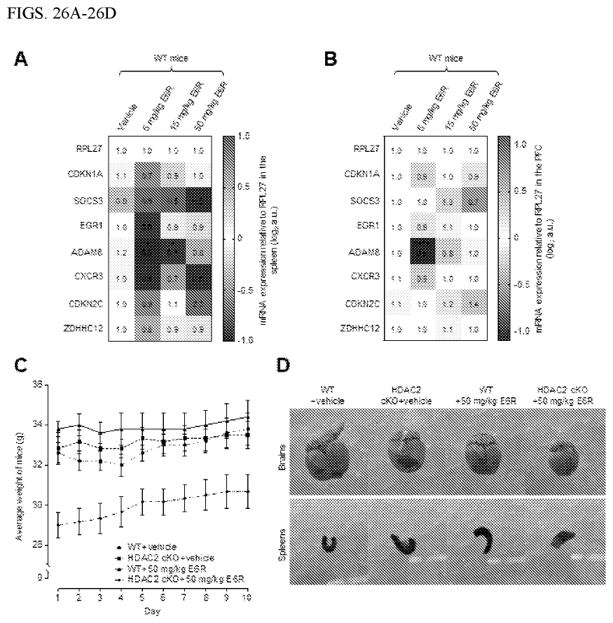

To check if E6R could affect transcriptional regulation in the mouse frontal cortex, expression changes of seven genes were first measured, by RT-qPCR, after chronically treating the animals with different doses of the compound, i.e. 5, 15, or 50 mg/kg (three to four mice per group), according to the protocol described in FIG. 9D. As a control, transcriptional perturbations were also examined in the spleen. In both cases, expression of the HDAC target genes (calculated relative to the RPL27 housekeeping gene [Beliakova-Bethell et al 2019]) used in the neuroblastoma experiments was checked (FIG. 8P), along with four genes known to be upregulated in the mouse frontal cortex upon SAHA treatment [de la Fuente Revenga et al 2018]. As shown in FIGS. 26A-B for the tested genes, E6R was indeed able to change expression levels in the spleen as well as in the frontal cortex of the treated animals.

To evaluate the global effect of E6R on transcriptional regulation in the frontal cortex, the highest tested dose from the pilot experiment was selected, i.e. 50 mg/kg, to conduct RNA-Seq. Based on the average body weight of the mice (˜30 g), this dose corresponded to an injection of ˜150 μM E6R. Importantly, this concentration was comparable to that used in published studies [Guan et al 2009, Nishikawa et al 2016, de la Fuente Revenga et al 2018], and was well-tolerated by the animals since they all behaved normally and followed a standard weight increase throughout the entire experiment (FIG. 26C). In addition, the morphologies of the brains and spleens remained highly similar between the various treatments (FIG. 26D). After chronically treating the different animals (five or six per group; FIGS. 9D-E) over ten days, and scarifying them 24 hours later, RNA from FC samples of WT or HDAC2 cKO mice treated or not with 50 mg/kg E6R were sequenced. As highlighted in FIG. 9F, E6R perturbed the transcriptome in the mouse FC much more specifically than the conditional depletion of HDAC2: 39 versus ˜3,600 genes with altered expression (relative to WT+vehicle), respectively. In addition, it was observed a significant overlap between genes differentially expressed upon E6R treatment in WT mice, and those altered in the HDAC2 cKO condition, which indicates E6R is targeting a subset of the HDAC2-containing complexes (FIG. 9G). These results are in good correlation with those obtained for the neuroblastoma cells (FIGS. 8S-U).

Remarkably, among the 39 differentially expressed genes upon treatment with 50 mg/kg E6R, Homer1 was downregulated by the compound compared to the WT+vehicle condition (FIG. 9H). The downregulation of Homer1 was also observed in the FC of HDAC2 cKO+vehicle as compared to the WT+vehicle group, indicating E6R phenocopied this condition (FIG. 9I). Moreover, the smaller fraction of differentially expressed genes in HDAC2 cKO mice treated or not with E6R supported that the effect of the compound is HDAC2 dependent (FIG. 9J). These observations were particularly interesting since Homer1 is a glutamate receptor whose function is altered in many neuropsychiatric disorders [Szumlinski et al 2005, Datko et al 2017]. In particular, Homer1 was reported to be upregulated in schizophrenia patients and rat models [Matosin et al 2016, Elfving et al 2019], suggesting that compounds decreasing Homer1 expression could potentially lead to beneficial therapeutic outcomes for this disease. In addition, Homer1 transcription was also shown to be under control of a SIN3A-containing HDAC complex in the mouse brain [Bridi et al 2020], which suggests that the E6R-mediated downregulation of Homer1 might originate from the binding of the compound to the PAH2 domain of SIN3A.

To check if E6R was indeed affecting recruitment of the SIN3A-containing HDAC complex at Homer1 in the mouse FC, alterations of SIN3A occupancy at the promoter of that gene were measured, by ChIP-qPCR. Pull-downs using anti-histone 3 (H3), and anti-IgG antibodies served as positive and negative controls, respectively, while SIN3A, the target of E6R, was pull-down using a well-established anti-SIN3A antibody [Dai et al 2017]. As indicated in FIG. 9K, compared to the vehicle condition, E6R significantly reduced recruitment of the SIN3A-containing HDAC complex in three different regions of Homer1's promoter, which validated the PPI inhibition mechanism of action of this compound in the mouse FC. This effect was not observed in HDAC2 cKO mice, indicating that a functional HDAC2-containing complex is required to detect the effect of E6R (FIG. 9L).

Finally, on the basis of these findings, it was decided to check if chronic E6R treatment could revert psychosis-like behaviors induced by psychedelic drugs in mice. To achieve this, the exacerbated head-twitch behavior of animals in response to DOI administration was examined (FIG. 9M) using an automated detection system [de la Fuente Revenga et al 2019, de la Fuente Revenga et al 2020]. Indeed, head twitching response (HTR), i.e. the side-to-side movement of the head, is the behavioral signature of psychedelic drugs upon stimulation of the serotonin 5-HT_{2A} receptor (5-HT_{2A}R), and has been widely used, among other assays in rodents, to model psychosis-like behavior [González-Maeso et al 2007, Halberstadt et al 2020]. In particular, it was reported that chronic SAHA treatment reduced DOI-induced head-twitch behavior in WT, but not in HDAC2 cKO mice [Kurita et al 2012, de la Fuente Revenga et al 2018]. This suggests that disrupting HDAC complexes with small molecules in the brain might potentially lead to protective phenotypes against psychotic behaviors. Following the protocol presented in FIG. 9N, HTR for WT or HDAC2 cKO mice treated or not with 50 mg/kg E6R were monitored. As shown in FIG. 9O, the DOI-induced HTR was significantly reduced upon treatment with E6R (p=0.012 for pre-versus post-DOI administration). Remarkably, this E6R-induced HTR decrease phenocopied the HDAC2 cKO condition before chronic treatment (FIG. 9P). Moreover, in agreement with FIGS. 9J and 9L, when the same group of HDAC2 cKO animals were then chronically treated with 50 mg/kg E6R, no additive effect was observed compared to untreated HDAC2 cKO mice, which indicates that E6R requires functional HDAC2-containing complexes to be active in vivo. Altogether, these in vivo results confirm the initial trends observed in yeast and mammalian cells, and highlight a higher level of effective specificity for our new HDAC complex PPI inhibitor, E6R.

### Example 10. E6R Reduces Neuroblastoma Cell Invasion and Tumor Growth in Mouse with Fewer Transcriptomic Changes

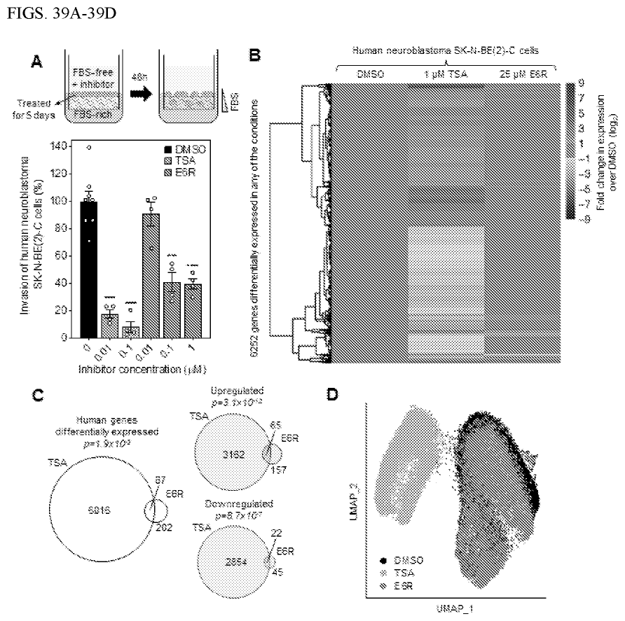

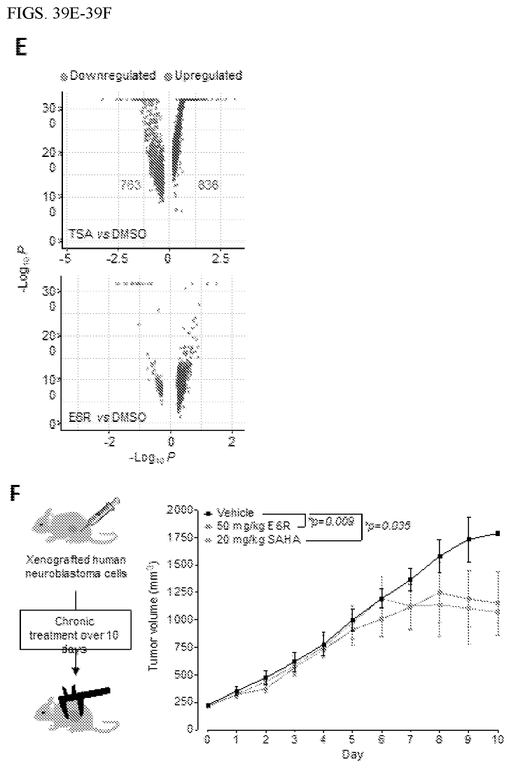

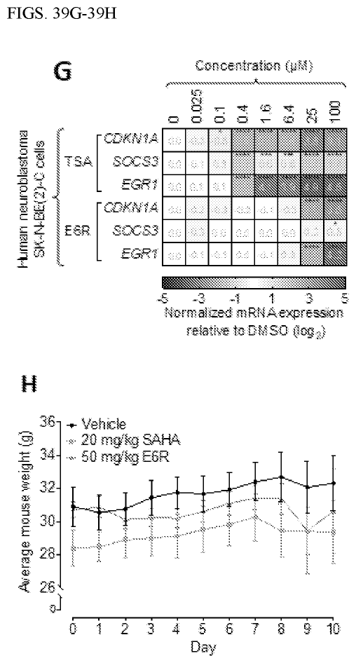

In order to check if E6R could produce similar biological effects as TSA, ability to inhibit human neuroblastoma SK-N-BE(2)-C cell invasion was tested. This phenotype widely used to test HDAC inhibitors [Y.-J Kwon et al 2015, Phimmachanh et al 2020]. We indeed observed that, similarly to TSA, E6R could significantly reduce the migration of neuroblastoma cells in the invasion assay (FIG. 39A).

To evaluate if E6R would confer more specific transcriptomic changes than enzymatic HDAC inhibitors such as TSA, perturbations induced by these compounds in the same human neuroblastoma SK-N-BE(2)-C cells were measured. Based on titration experiments using three SIN3 HDAC complex-regulated genes (FIG. 39G), cells were treated with 1 μM TSA or 25 μM E6R to conduct RNA-seq. The replicates of each condition clustered together, showing high reproducibility of the treatments (FIG. 25). Remarkably, E6R perturbed expression of approximately 20-fold fewer human genes compared to TSA (FIG. 8R and FIG. 39B). Significant overlaps between genes differentially expressed upon TSA or E6R treatments were observed, suggesting that E6R targets only a subset of the TSA-sensitive genes (FIG. 4C).

To further assess the transcriptomic states of human neuroblastoma SK-N-BE(2)-C cells treated with DMSO control, TSA or E6R, single-cell (sc) RNA-seq analyses were conducted. By performing dimensionality reduction and unbiased clustering on the resulting data, it was confirmed that E6R yielded more subtle transcriptomic alterations than TSA since TSA-treated cells clustered distinctly and E6R-treated cells admixed with the DMSO control (FIG. 39D) while perturbing expression of fewer genes (FIG. 39E). Interestingly, among the 65 upregulated genes by both TSA and E6R in the bulk RNA-seq approach (FIG. 39C), 43 and 22 (21 common) were also upregulated by these compounds in the scRNA-seq experiments, respectively. In addition, among the 22 downregulated genes (FIG. 39C), 13 and 5 (3 common) were also downregulated by TSA and E6R by scRNA-seq, respectively.

Finally, to examine the effect of E6R in vivo, a three-arm efficacy study was performed on immunocompromised mice xenografted with human neuroblastoma SK-N-BE(2)-C cells. To compare the effect of an established HDAC enzymatic inhibitor to that of E6R, suberoylanilide hydroxamic acid (SAHA) was used, which is a structural analog of TSA reported to decrease neuroblastoma growth in vivo [Rettig et al 2015] and tested in clinical trials (www.ClinicalTrials.gov identifiers: NCT01208454, NCT03332667). Both compounds were well-tolerated by the animals and did not affect their weight over time (FIG. 39H). In this assay, E6R and SAHA both significantly reduced the growth of neuroblastoma tumors compared to the vehicle in the xenografted mice (FIG. 39F), which illustrated the potential for medical applications of this compound.

### Example 11. E6R Decreases a Psychosis Behavior with Fewer Transcriptomic Changes in the Mouse Frontal Cortex

Previous findings also reported that repeated administration of SAHA affects transcription of numerous plasticity-related genes in the frontal cortex (FC) and reduces head-twitch response (HTR) behavior [Kurita et al 2012, de la Fuente Revenga et al 2018], a mouse behavioral proxy of human hallucinogen effects. These SAHA-dependent transcriptomic and behavioral effects were not observed in Hdac2 conditional knockout mice (Hdac2 cKO) with selected deletion of Hdac2 in the pyramidal neurons [de la Fuente Revenga 2018]. Based on these findings, it was tested whether chronic treatment with 50 mg/kg E6R could revert the psychedelic-induced HTR behavior. To achieve this, we examined HTR in mice exposed to the psychedelic drug 2,5-dimethoxy-4-iodoamphetamine (DOI) [de la Fuente Revenga et al 2020], following repeated treatment with E6R (FIG. 9M). We observed that E6R significantly reduced DOI-induced HTR in WT mice (FIG. 9O), to the same extent than in untreated Hdac2 cKO animals (FIG. 9O-P). Moreover, when the same group of Hdac2 cKO mice was chronically treated with E6R, no additive effect was observed (FIG. 5C), indicating that E6R is epistatic to Hdac2 cKO in the FC and thus requires functional HDAC2-containing complexes to reduce the HTR behavior.

To measure the impact of E6R on HDAC2-dependent transcriptional regulation in the FC, we conducted RNA-seq on FC tissue samples from WT or Hdac2 cKO mice chronically treated with vehicle or 50 mg/kg E6R for ten days. Compared to conditional Hdac2 deletion, treatment with E6R led to approximately 80-fold fewer transcriptomic changes: only 48 genes were significantly affected in the FC of E6R-treated mice compared to 3,995 genes in the Hdac2 cKO condition (FIG. 9Q). Importantly, the majority of the differentially expressed genes following treatment with E6R were also altered in Hdac2 cKO mice, indicating that this compound affects only a subset of the HDAC2-containing complexes in the FC (FIG. 9R). In addition, gene expression changes induced by E6R treatment were markedly muted in the Hdac2 cKO background as compared to the WT background, suggesting that the E6R mechanism of action requires functional HDAC2 complexes (FIG. 9S). Together, these results are in agreement with those obtained in human neuroblastoma cells and confirm that E6R can achieve HDAC inhibitor-like effects in vivo while inducing only a few specific transcriptional changes.

Finally, to confirm that the E6R-mediated perturbations of gene regulatory functions in vivo results from inhibition of the SIN3 HDAC complex recruitment, we analyzed, by ChIP-qPCR, SIN3A occupancy at the Homer1 locus in FC tissues. Homer1 is an established SIN3A-containing HDAC complex-regulated gene [Bridi et al 2020], whose function is altered in many neuropsychiatric disorders such as schizophrenia where it is upregulated in brain tissues [Matosin et al 2016, Elfving et al 2019]. Thus, direct downregulation of this gene following treatment with an inhibitor might potentially have beneficial therapeutic effects. SIN3A ChIP-qPCR analyses confirmed that E6R significantly reduces the presence of SIN3A at the promoter of Homer1 (FIG. 5T). This disruption was not observed in Hdac2 cKO mice, indicating that a functional HDAC2-containing complex is required to detect the effect of the compound (FIG. 5U). Together, those results strongly suggest that the biological activity and the highly selective transcriptomic effects of E6R originate from its ability to bind to the PAH2 domains of SIN3A/B proteins, which, in turn, only perturbs a subset of the SIN3 HDAC sub-complexes traditionally affected via HDAC enzymatic inhibition.

### Discussion

So far, enzymatic complexes such as those involving HDACs have mostly been disrupted by small molecule perturbants that target their catalytic component(s), rather than by affecting other functionalities. Such enzymatic inhibitors do not usually differentiate between distinct HDAC complexes, and their uses have thus possibly impaired our understanding of the normal, and disease biology of specific HDAC-related processes. Analysis of the reported subunits of a model HDAC complex, the yeast S. cerevisiae Rpd3L HDAC, suggests that, not only the HDAC enzyme, but also other subunits (often less well characterized) play key roles in transcriptional regulation at different loci. In particular, interrogating all functionalities of the Rpd3L HDAC complex with a focused, yet agnostic chemical screening approach has revealed that the most potent functional small molecule perturbants do not act as enzymatic inhibitors.

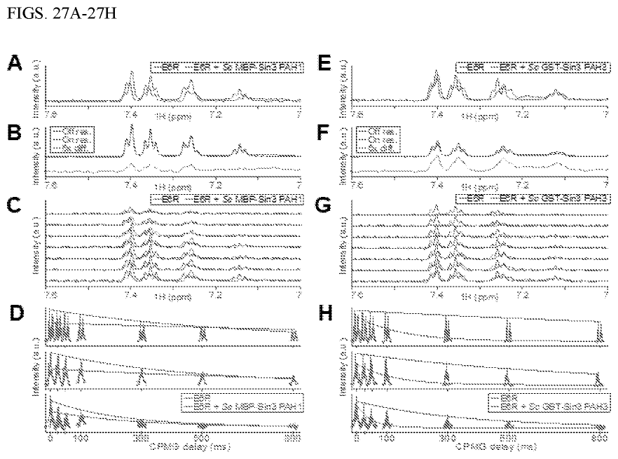

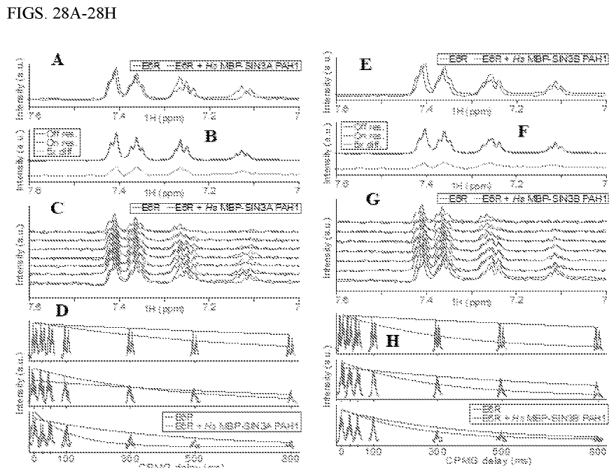

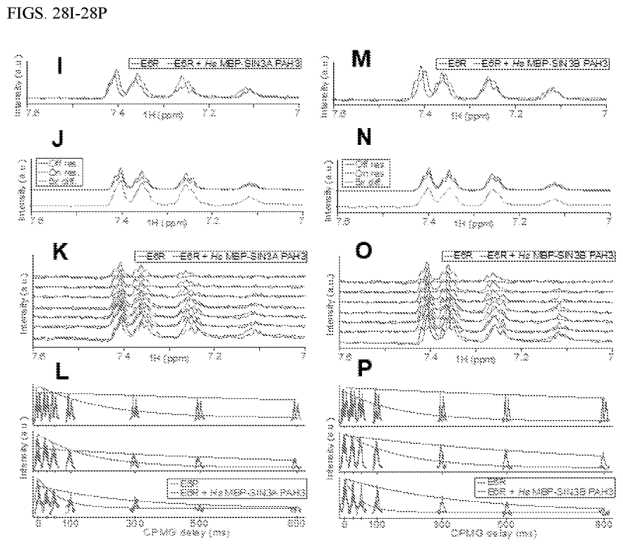

However, our approach also includes testing the resulting hits from screening against binary PPIs, an under-explored class of targets. In contrast, the number of PPIs identified in various organisms has constantly been growing over the last years, and so have the interaction maps of their cellular complexes and pathways. Information can now easily be extracted from online databases and potential PPI targets can thus be quickly prioritized. In addition, high-throughput binary interaction assays allow reconstituting PPIs in various experimental settings, where candidate compounds can directly be tested for their PPI inhibition activity. Such activity can then be confirmed using well-established techniques such as ChIP-Seq, XRD, etc. This has allowed us to identify E6, and its enantiomer E6R. These molecules can bind to the PAH2 domain of Sin3, a scaffold protein of the Rpd3L HDAC complex, and break its interactions with transcription factors such as Ume6. These new small molecule perturbants should thus expand the current panel of chemical probes for HDAC complexes, and potentially transform our understanding of the normal and disease biology of specific HDAC processes, as opposed to broadly affecting enzymatic activity by binding the catalytic pocket of HDACs. On the basis of the small SAR study conducted here, analogs of E6R should also be synthesized and tested in the future, to increase the potency and specificity of its chemical scaffold. In particular, it was observed, by protein NMR, that E6R can bind other PAH domains of yeast S. cerevisiae Sin3 (FIG. 27), and human SIN3A and SIN3B (FIG. 28). This means a new classes of SIN3 inhibitors could be developed that are more selective towards one PAH domain, engaged in specific PPIs, while leaving other PAH domains, engaged in other PPIs, unperturbed.

Although E6 was originally identified in a yeast-based system, this compound is also functional in mammalian cells and mouse models, with increased specificity compared to known enzymatic inhibitors, which, again, establishes yeast as a suitable model organism to discover new mechanisms and chemical probes in high-throughput settings. Many blockbuster drugs used in cancer treatments (e.g. rapamycin or cis-platin) or other diseases (e.g. statins for treatment of cholesterol) hit orthologous targets in yeast and human. Importantly, even though all efforts were focused on characterizing E6 in the present study, six other verified hits also had interesting effects in both, yeast and mammalian cells. Completing mapping of the complex with orthogonal binary interaction assays will provide a more comprehensive set of PPI targets to test against compounds [Choi et al 2019]. In addition, considering subunits involved in transcriptional activation mechanisms (e.g. complexes involving histone acetyl transferase enzymes) will likely release valuable information, since some of our small molecules appear to have opposite effects on transcription, and directly decrease the basal expression levels of HDAC target genes at particular loci (e.g. effect of E6R at Homer1 in the mouse FC).

Finally, the new SIN3 inhibitor identified here, E6R, led to similar biological effects as classical enzymatic inhibitors, while inducing fewer transcriptomic perturbations in mammalian cells and mice. In short, E6R has an “oligogenic” effect on the transcriptome, resulting in reduced toxicity and fewer side effects during treatments of disorders in which HDAC complexes play a role. One of the major drawbacks in using currently available HDAC enzymatic inhibitors is that they confer “multigenic” effects on the transcriptome since they affect the transcriptional levels of hundreds to thousands of genes, which can hinder their use in treating most human pathologies, with the exception of a few blood cancers. In short, as exemplified here for E6R in a psychosis mouse model, there might be some advantages in using “oligogenic” compounds instead of “multigenic” compounds, such as classical enzymatic HDAC inhibitors, to better dissect and understand biological processes and expand the clinical use of HDAC complex inhibitors.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

> Armache K-J, et al. Structures of Complete RNA Polymerase II and Its Subcomplex, Rpb4/7. J Biol Chem. 280(8), 7131 (2005)
> Balzi E, Goffeau A. Yeast multidrug resistance: The PDR network. J Bioenerg Biomembr. 27, 71 (1995)
> Banks CAS, et al. Integrative modeling of a Sin3/HDAC complex sub-structure. Cell Rep. 31(2), 107516 (2020)
> Beliakova-Bethell N, et al. Histone deacetylase inhibitors induce complex host responses that contribute to differential potencies of these compounds in HIV reactivation. J Biol Chem. 294(14), 5576 (2019)
> Braun P, et al. An experimentally derived confidence score for binary protein-protein interactions. Nat Methods. 6, 91 (2009)
> Bridi M, et al. Transcriptional corepressor SIN3A regulates hippocampal synaptic plasticity via Homer1/mGluR5 signaling. JCI Insight. 5(5), e92385 (2020)
> Brown L E, et al. Discovery of new antimalarial chemotypes through chemical methodology and library development. Proc Natl Acad Sci USA. 108, 6775 (2011)
> Brubaker K, et al. Solution structure of the interacting domains of the Mad-Sin3 complex: implications for recruitment of a chromatin-modifying complex. Cell. 103, 655 (2000)
> Calderone A, et al. mentha: a resource for browsing integrated protein-interaction networks. Nat Methods. 10(8), 690 (2013)
> Cassonnet P, et al. Benchmarking a luciferase complementation assay for detecting protein complexes. Nat Methods. 8(12), 990 (2011)
> Cer R Z, et al. IC₅₀-to-Kᵢ: a web-based tool for converting IC₅₀ to Kᵢ values for inhibitors of enzyme activity and ligand binding. Nucleic Acids Res. 37, W441 (2009)
> Choi S G, et al. Maximizing binary interactome mapping with a minimal number of assays. Nat Commun. 10, 3907 (2019)
> Dai Q, et al. Striking a balance: regulation of transposable elements by Zfp281 and M112 in mouse embryonic stem cells. Nucleic Acids Res. 45(21), 12301 (2017)
> Daily K, et al. MotifMap: integrative genome-wide maps of regulatory motif sites for model species. BMC Bioinformatics. 12, 495 (2011)
> Datko M C, et al. Behavioral and neurochemical phenotyping of mice incapable of Homer1a induction. Front Behav Neurosci. 11, 208 (2017)
> de la Fuente Revenga M, et al. Automated quantification of head-twitch response in mice via ear tag reporter coupled with biphasic detection. J Neurosci Methods. 334, 108595 (2020)
> de la Fuente Revenga M, et al. HDAC2-dependent Antipsychotic-like Effects of Chronic Treatment with the HDAC Inhibitor SAHA in Mice. Neurosci. 388, 102 (2018)
> Dubois E, et al. Specific induction of catabolism and its relation to repression of biosynthesis in arginine metabolism of Saccharomyces cerevisiae. J Mol Biol. 122(4), 383 (1978)
> Elfving B, et al. Differential expression of synaptic markers regulated during neurodevelopment in a rat model of schizophrenia-like behavior. Prog Neuropsychopharmacol Biol Psychiatry. 95, 109669 (2019)
> Fischle W, et al. Enzymatic activity associated with class II HDACs is dependent on a multiprotein complex containing HDAC3 and SMRT/N-CoR. Mol Cell. 9(1), 45 (2002)
> Frumm S M, et al. Selective HDAC1/HDAC2 Inhibitors Induce Neuroblastoma Differentiation. Cell Chem Biol. 20, 713 (2013)
> Glaser K B, et al. Gene expression profiling of multiple histone deacetylase (HDAC) inhibitors: defining a common gene set produced by HDAC inhibition in T24 and MDA carcinoma cell lines. Mol Cancer Ther. 2, 151 (2003)
> González-Maeso J, et al. Hallucinogens recruit specific cortical 5-HT(2A) receptor-mediated signaling pathways to affect behavior. Neuron. 53(3), 439 (2007)
> Grigat M, et al. Multiple histone deacetylases are recruited by corepressor Sin3 and contribute to gene repression mediated by Opi1 regulator of phospholipid biosynthesis in the yeast Saccharomyces cerevisiae. Mol Genet Genomics. 287(6), 461 (2012)
> Guan J-S, et al. HDAC2 negatively regulates memory formation and synaptic plasticity. Nature. 459(7243), 55 (2009)
> Halberstadt A L, et al. Correlation between the potency of hallucinogens in the mouse head-twitch response assay and their behavioral and subjective effects in other species. Neuropharmacology. 167, 107933 (2020)
> Hamon V, et al. 2P2Ichem: focused chemical libraries dedicated to orthosteric modulation of protein-protein interactions. MedChemComm. 4, 797 (2013)
> Hanks J B, González-Maeso J. Animal Models of Serotonergic Psychedelics. ACS Chem Neurosci. 4(1), 33 (2013)
> Hein M Y, et al. A human interactome in three quantitative dimensions organized by stoichiometries and abundances. Cell. 163, 712 (2015)
> Hsu C-W, et al. Identification of HDAC inhibitors using a cell-based HDAC I/II assay. J Biomol Screen. 21(6), 643 (2016)
> Huttlin E L, et al. The BioPlex Network: A Systematic Exploration of the Human Interactome. Cell. 162,425 (2015)
> Ibi D, et al. Antipsychotic-induced Hdac2 transcription via NF-κB leads to synaptic and cognitive side effects. Nat Neurosci. 20(9), 1247 (2017)
> Icardi L, et al. The Sin3a repressor complex is a master regulator of STAT transcriptional activity. Proc Natl Acad Sci USA. 109(30), 12058 (2012)
> Ji Q, et al. CRL4B interacts with and coordinates the SIN3A-HDAC complex to repress CDKN1A and drive cell cycle progression. J Cell Sci. 127, 4679 (2014)
> Kadosh D, Struhl K. Repression by Ume6 Involves Recruitment of a Complex Containing Sin3 Corepressor and Rpd3 Histone Deacetylase to Target Promoters. Cell. 89(3), 365 (1997)
> Kemmeren P, et al. Large-scale genetic perturbations reveal regulatory networks and an abundance of gene-specific repressors. Cell. 157(3), 740 (2014)
> Kumar, G S et al. Solution structure of the mSin3A PAH2-Pf1 SID1 complex: A Mad1/Mxd1-like interaction disrupted by MRG15 in the Rpd3S/Sin3S complex. J Mol. Biol. 408, 987-1000 (2011).
> Kurita M, et al. HDAC2 regulates atypical antipsychotic responses through the modulation of mGlu2 promoter activity. Nat Neurosci. 15(9), 1245 (2012)
> Kwon Y-J, et al. Selective Inhibition of SIN3 Corepressor with Avermectins as a Novel Therapeutic Strategy in Triple-Negative Breast Cancer. Mol Cancer Ther. 14(8), 1824 (2015)
> Lamberto I, et al. Structure-Guided Development of a Potent and Selective Non-covalent Active-Site Inhibitor of USP7. Cell Chem Biol. 24, 1490 (2017)
> Le Guezennec X, et al. Molecular characterization of Sin3 PAH-domain interactor specificity and identification of PAH partners. Nucleic Acids Res. 34(14), 3929 (2006)
> Li Y, et al. Zinc-dependent Deacetylase (HDAC) Inhibitors with Different Zinc Binding Groups. Curr Top Med Chem. 19(3), 223 (2019)
> Lievens S, et al. Kinase Substrate Sensor (KISS), a Mammalian In Situ Protein Interaction Sensor. Mol Cell Proteom. 13(12), 3332 (2014)
> Luck K, et al. A reference map of the human binary protein interactome. Nature. 580, 402 (2020)
> Matosin N, et al. Molecular evidence of synaptic pathology in the CA1 region in schizophrenia. NPJ Schizophr. 2, 16022 (2016)
> Millard C J, et al. Targeting class I histone deacetylases in a “complex” environment. Trends Pharmacol Sci. 38(4), 363 (2017)
> Nichols D E. Psychedelics. Pharmacol Rev. 68(2), 264 (2016)
> Nishikawa J L, et al. Inhibiting fungal multidrug resistance by disrupting an activator-mediator interaction. Nature. 530(7591), 485 (2016)
> Pal S, et al. Impaired cohesion and homologous recombination during replicative aging in budding yeast. Sci Adv. 4, eaaq0236 (2018)
> Phimmachanh, M et al. Histone Deacetylases and Histone Deacetylase Inhibitors in Neuroblastoma. Front. Cell. Dev. Biol. 8, (2020)
> Piecuch A, Oblak E. Yeast ABC proteins involved in multidrug resistance. Cell Mol Biol Lett. 19, 1 (2014)
> Rettig, I et al. Selective inhibition of HDAC8 decreases neuroblastoma growth in vitro and in vivo and enhances retinoic acid-mediated differentiation. Cell Death Dis. 6, e1657 (2015)
> Robinson J T, et al. Integrative Genomics Viewer. Nat Biotechnol. 29, 24 (2011)
> Rossi M J, et al. Simplified ChIP-exo assays. Nat Commun. 9, 2842 (2018)
> Sardiu M E, et al. Determining Protein Complex Connectivity Using a Probabilistic Deletion Network Derived from Quantitative Proteomics. PLoS ONE. 4(10), e7310 (2009)
> Sardiu M E, et al. Suberoylanilide Hydroxamic Acid (SAHA)-Induced Dynamics of a Human Histone Deacetylase Protein Interaction Network. Mol Cell Proteom. 13, 3114 (2014)
> Frumm, S M et al. Selective HDAC1/HDAC2 inhibitors induce neuroblastoma differentiation. Chem. Biol. 20, 713-725 (2013)
> Strich R, et al. UME6 is a key regulator of nitrogen repression and meiotic development. Genes Dev. 8, 796 (1994)
> Szumlinski K K, et al. Behavioral and neurochemical phenotyping of Homer1 mutant mice: possible relevance to schizophrenia. Genes Brain Behav. 4(5), 273 (2005)
> Teste M-A, et al. Validation of reference genes for quantitative expression analysis by real-time RT-PCR in Saccharomyces cerevisiae. BMC Mol Biol. 10, 99 (2009)
> Trepte P, et al. A quantitative mapping approach to identify direct interactions within complexomes. bioRxiv, 26 Aug. 2021.
> van Ingen H, et al. Role of Structural and Dynamical Plasticity in Sin3: The Free PAH2 Domain is a Folded Module in mSin3B. J Mol Biol. 358, 485 (2006)
> Vidal M, Gaber R F. RPD3 encodes a second factor required to achieve maximum positive and negative transcriptional states in Saccharomyces cerevisiae. Mol Cell Biol. 11(12), 6317 (1991)
> Vidal M, et al. Direct selection for mutants with increased K⁺ transport in Saccharomyces cerevisiae. Genetics. 125(2), 313 (1990)
> Vidal M, et al. RPD1 (SIN3/UME4) is required for maximal activation and repression of diverse yeast genes. Mol Cell Biol. 11(12), 6306 (1991)
> Vidal M, et al. Reverse two-hybrid and one-hybrid systems to detect dissociation of protein-protein and DNA-protein interactions. Proc Natl Acad Sci USA. 93, 10315 (1996)
> Vidal M. Playing hide-and-seek with yeast. Cell. 166(5), 1069 (2016)
> Vogelauer M, et al. Global histone acetylation and deacetylation in yeast. Nature. 408(6811), 495 (2000)
> Wan C, et al. Panorama of ancient metazoan macromolecular complexes. Nature. 525, 339 (2015)
> Washburn B K, Esposito R E. Identification of the Sin3-binding site in Ume6 defines a two-step process for conversion of Ume6 from a transcriptional repressor to an activator in yeast. Mol Cell Biol. 21(6), 2057 (2001)
> YeRI project. The Yeast Protein Interactome Mapping Project: http://yeri.baderlab.org/. May 2020
> Yoshida, M et al. Trichostatin A and trapoxin: novel chemical probes for the role of histone acetylation in chromatin structure and function. Bioessays 17, 423-430 (1995)
> Zhan C, et al. Identification of reference genes for qRT-PCR in human lung squamous-cell carcinoma by RNA-Seq. Acta Biochim Biophys Sin. 46(4), 330 (2014)

### Materials and Methods:

> Adams P D, et al. PHENIX: a comprehensive Python-based system for macromolecular structure solution. Acta Cryst. D 66, 213 (2010)
> Andrews S. FastQC: A quality control tool for high throughput sequence data. Available online: https://www.bioinformatics.babraham.ac.uk/projects/fastqc/(2010)
> Baell J B, Holloway G A. New Substructure Filters for Removal of Pan Assay Interference Compounds (PAINS) from Screening Libraries and for Their Exclusion in Bioassays. J Med Chem. 53(7), 2719 (2010)
> Baganz, F. et al. Suitability of replacement markers for functional analysis studies in Saccharomyces cerevisiae. Yeast 13, 1563-1573 (1997)
> Barski A, et al. High-resolution profiling of histone methylations in the human genome. Cell. 129(4), 823 (2007)
> Benjamini Y, Hochberg Y. Controlling the false discovery rate: A practical and powerful approach to multiple testing. J R Statist Soc B. 57, 289 (1995)
> Bolger A M, et al. Trimmomatic: A flexible trimmer for illumina sequence data. Bioinformatics. 30, 2114 (2014)
> Calderone, A, et al. Mentha: a resource for browsing integrated protein-interaction networks. Nat Methods 10, 690-691 (2013)> Chen E Y, et al. Enrichr: Interactive and collaborative HTML5 gene list enrichment analysis tool. BMC Bioinformatics. 14, 128 (2013)
> Choi S G, et al. G proteins and autocrine signaling differentially regulate gonadotropin subunit expression in pituitary gonadotrope. J Biol Chem. 287(25), 21550 (2012)
> Choi, S G, et al. Maximizing binary interactome mapping with a minimal number of assays. Nat. Commun. 10, 3907 (2019)> de la Fuente Revenga M, et al. Fully automated head-twitch detection system for the study of 5-HT_{2A} receptor pharmacology in vivo. Sci Rep. 9, 14247 (2019)
> Emsley P, Cowtan K. Coot: model-building tools for molecular graphics. Acta Cryst. D 60, 2126 (2004)
> Gardner J M, Jaspersen S L. Manipulating the yeast genome: deletion, mutation, and tagging by PCR. Methods Mol Biol. 1205, 45 (2014)
> Gierahn, T M, et al. Seq-Well: portable, low-cost RNA sequencing of single cells at high throughput. Nat Methods 14, 395-398 (2017)
> Guldener, U, et. al. A new efficient gene disruption cassette for repeated use in budding yeast. Nucleic Acids Res 24, 2519-2524 (1996)
> Hajduk P J, et al. One-Dimensional Relaxation- and Diffusion-Edited NMR Methods for Screening Compounds That Bind to Macromolecules. J Am Chem Soc. 119(50), 12257 (1997)
> Hyberts, S G, et al. Application of iterative soft thresholding for fast reconstruction of NMR data non-uniformly sampled with multidimensional Poisson Gap scheduling. J Biomol NMR 52, 315-327 (2012)
> Hughes, T K, et al. Second-Strand Synthesis-Based Massively Parallel scRNA-Seq Reveals Cellular States and Molecular Features of Human Inflammatory Skin Pathologies. Immunity 53, 878-894.e877 (2020)> Jezek M, et al. Chromatin Immunoprecipitation (ChIP) of Histone Modifications from Saccharomyces cerevisiae. J Vis Exp. 130, 57080 (2017)
> Kabsch W. Integration, scaling, space-group assignment and post-refinement. Acta Cryst. D66, 133 (2010)
> Keller, R L J, E T H Zurich (2005)> Kumar G S, et al. Solution Structure of the mSin3A PAH2-Pf1 SID1 Complex: a Mad1/Mxd1-like Interaction Disrupted by MRG15 in the Rpd3S/Sin3S Complex. J MolBiol. 408(5), 987 (2011)
> Liao Y, et al. The subread aligner: Fast, accurate and scalable read mapping by seed-and-vote. Nucleic Acids Res. 41(10), e108 (2013)
> Liao Y, et al. featureCounts: An efficient general purpose program for assigning sequence reads to genomic features. Bioinformatics. 30, 923 (2014)
> Loewith R, et al. Pho23 is associated with the Rpd3 histone deacetylase and is required for its normal function in regulation of gene expression and silencing in Saccharomyces cerevisiae. J Biol Chem. 276(26), 24068 (2001)
> Love M I, et al. Moderated estimation of fold change and dispersion for RNA-seq data with DESeq2. Genome Biol. 15, 550 (2014)
> Mayer M, Meyer B. Group Epitope Mapping by Saturation Transfer Difference NMR To Identify Segments of a Ligand in Direct Contact with a Protein Receptor. J Am Chem Soc. 123(25), 6108 (2001)
> McCoy A J, et al. Phaser crystallographic software. J Appl Cryst. 40, 658 (2007)
> Morris G M, et al. Autodock4 and AutoDockTools4: automated docking with selective receptor flexiblity. J Comput Chem. 16, 2785 (2009)
> Patro R, et al. Salmon provides fast and bias-aware quantification of transcript expression. Nat Methods. 14, 417 (2017)
> Robinson M D, et al. edgeR: A bioconductor package for differential expression analysis of digital gene expression data. Bioinformatics. 26, 139 (2010)
> Rossi, M J, et al. Simplified ChIP-exo assays. Nat. Commun. 9, 2842 (2018)
> Sanner M F. Python: A Programming Language for Software Integration and Development. J Mol Graphics Mod. 17, 57 (1999)
> Sertil O, et al. Direct role for the Rpd3 complex in transcriptional induction of the anaerobic DAN/TIR genes in yeast. Mol Cell Biol. 27(6), 2037 (2007)
> Shen, Y, et al. Protein backbone and sidechain torsion angles predicted from NMR chemical shifts using artificial neural networks. J Biomol NMR 56, 227-241 (2013)
> Soneson C, et al. Differential analyses for RNA-seq: transcript-level estimates improve gene-level inferences. F1000Res. 4, 1521 (2015)
> To E, et al. Isolation and characterization of acid phosphatase mutants in Saccharomyces cerevisiae. J Bacteriol. 113, 727 (1973)
> Teste, M A, et al. Validation of reference genes for quantitative expression analysis by real-time RT-PCR in Saccharomyces cerevisiae. BMC Mol Biol 10, 99 (2009)> Trott O, Olson A J. AutoDock Vina: improving the speed and accuracy of docking with a new scoring function, efficient optimization, and multithreading. J Comput Chem. 31(2), 455 (2010)
> Wendland, J. CR-based methods facilitate targeted gene manipulations and cloning procedures. Curr Genet 44, 115-123 (2003)
> Yu G, et al. clusterProfiler: An r package for comparing biological themes among gene clusters. OMICS. 16(5), 284 (2012)
> Yu H, et al. High-quality binary protein interaction map of the yeast interactome network. Science. 322, 104 (2008)
> Zhang Y, et al. Model-based Analysis of ChIP-Seq(MACS). Genome Biol, 9(9), R137 (2008)

We claim:

1. A method of disrupting protein interactions in a histone deacetylase (HDAC) complex in a cell of subject comprising administering to the subject an effective amount of a compound having the structure of Formula (I): 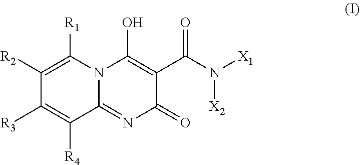
wherein
R₁, R₂, R₃, and R₄ are each independently selected from —H, halo, alkyl, alkenyl, alkynyl, alkoxy, and alkylamino;
X₁ is selected from optionally substituted alkyl, hydoxyalkyl, cycloalkyl, cycloheteroalkyl, aryl, heteroaryl, arylalkyl, and heteroarylalkyl; and
X₂ is selected from —H or alkyl,
or a pharmaceutically acceptable salt thereof.

2. The method claim 1, wherein the compound disrupts protein interactions in the HDAC complex without substantially inhibiting enzyme activity in the HDAC.

3. The method claim 1 or 2, wherein the compound inhibits the interactions between a scaffold protein and a transcription factor in the HDAC complex.

4. The method of any one of claims 1-3, wherein the HDAC complex comprises a SIN3 scaffold protein.

5. The method of claim 5, wherein the compound interacts by binding to a PAH domain of the SIN3 scaffold protein.

6. The method of any one of claims 1-5, wherein the HDAC complex comprises a MAD transcription factor.

7. The method of claim of any one of claims 1-5, wherein the HDAC complex comprises HDAC1 or HDAC2.

8. The method of any one of claims 1-7, wherein the HDAC complex is a Sin3-HDAC complex.

9. A method of treating a disease mediated by a histone deacetylase (HDAC) complex in a subject in need thereof comprising administering to the subject an effective amount of a compound having the structure of Formula (I): 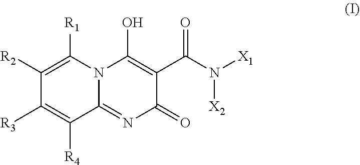
wherein
R₁, R₂, R₃, and R₄ are each independently selected from —H, halo, alkyl, alkenyl, alkynyl, alkoxy, and alkylamino;
X₁ is selected from optionally substituted alkyl, hydoxyalkyl, cycloalkyl, cycloheteroalkyl, aryl, heteroaryl, arylalkyl, and heteroarylalkyl; and
X₂ is selected from —H or alkyl,
or a pharmaceutically acceptable salt thereof.

10. The method claim 9, wherein the compound disrupts protein interactions in the HDAC complex.

11. The method claim 9, wherein the compound disrupts protein interactions in the HDAC complex without substantially inhibiting enzyme activity in the HDAC.

12. The method claim 10 or 11, wherein the compound inhibits the interactions between a scaffold protein and a transcription factor in the HDAC complex.

13. The method of any one of claims 9-12, wherein the HDAC complex comprises a SIN3 scaffold protein.

14. The method of claim 13, wherein the compound interacts by binding to a PAH domain of the SIN3 scaffold protein.

15. The method of any one of claims 9-14, wherein the HDAC complex comprises a MAD transcription factor.

16. The method of claim of any one of claims 9-15, wherein the HDAC complex comprises HDAC1 or HDAC2.

17. The method of any one of claims 9-16, wherein the HDAC complex is a Sin3-HDAC complex.

18. The method of any one of claims 9-17, wherein the disease mediated by a histone deacetylase (HDAC) complex is selected from a cancer, a neurodegenerative disease, and a mental disorder.

19. The method of claim of claim 18, wherein the cancer is selected from triple negative breast cancer, T-cell lymphoma, multiple myeloma, a neuroblastoma, and a medulloblastoma.

20. The method of claim of claim 18, wherein the neurodegenerative disease is Huntington's disease.

21. The method of claim 18, wherein the mental disorder is selected from psychosis, schizophrenia, and bipolar disorder.

22. The method of any one of claims 1-21, wherein the compound has the structure: 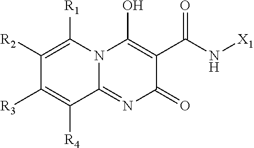
wherein
R₁, R₂, R₃, and R₄ are each independently selected from —H, halo, alkyl, alkenyl, alkynyl, alkoxy, and alkylamino;
X₁ is selected from optionally substituted alkyl, hydoxyalkyl, cycloalkyl, cycloheterocyclyl, aryl, heteroaryl, arylalkyl, and heteroarylalkyl,
or a pharmaceutically acceptable salt thereof.

23. The method of claim 22, wherein R₁, R₂, R₃, and R₄ are each independently selected from —H and alkyl.

24. The method of claim 23, wherein R₁, R₂, R₃, and R₄ are each independently selected from —H and —CH₃.

25. The method of claim 24, wherein one of R₁, R₂, R₃, and R₄ is —CH₃.

26. The method of claim 24, wherein each of R₁, R₂, R₃, and R₄ is —H.

27. The method of any one of claims 22-26, wherein X₁ is optionally substituted alkyl or hydoxyalkyl.

28. The method of claim 27, wherein X₁ is (C₁-C₆) alkyl.

29. The method of claim 28, wherein X₁ is a branched (C₁-C₆) alkyl.

30. The method of claim 27, wherein X₁ is (C₁-C₆) hydoxyalkyl.

31. The method of claim 30, wherein X₁ is a branched (C₁-C₆) hydoxyalkyl.

32. The method of any one of claims 22-26, wherein X₁ is optionally substituted cycloalkyl.

33. The method of claim 32, wherein X₁ is selected from optionally substituted cyclopentyl, cyclohexyl, and cycloheptyl.

34. The method of claim 33, wherein the cyclopentyl, cyclohexyl, or cycloheptyl is unsubstituted.

35. The method of claim 33, wherein the cyclopentyl, cyclohexyl, or cycloheptyl is substituted with at least one alkyl group.

36. The method of claim 35, wherein the alkyl group is a methyl group.

37. The method of any one of claims 27-36, wherein X₁ is selected from

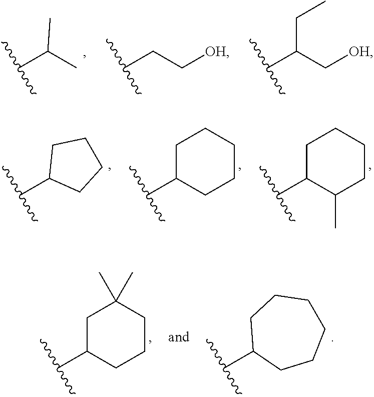

38. The method of any one of claims 22-26 wherein X₁ is an optionally substituted arylalkyl.

39. The method of claim 38, wherein X₁ has the structure: 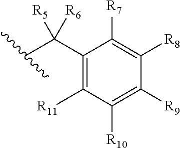
wherein
R₅ and R₆ are each independently selected from —H and alkyl; and
R₇, R₈, R₉, R₁₀, and R₁₁ are each independently selected from —H, halo, alkyl, alkenyl, alkynyl, alkoxy, and alkylamino.

40. The method of claim 39, wherein R₅ and R₆ are each —H.

41. The method of claim 39, wherein one of R₅ and R₆ is H and the other is —CH₃.

42. The method of claim 40 or 41, wherein X₁ is selected from

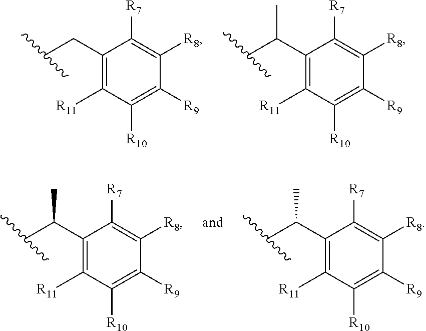

43. The method of claim 42, wherein R₇, R₈, R₉, R₁₀, and R₁₁ are each independently selected from —H, halo, or alkoxy.

44. The method of claim 43, wherein R₇, R₈, R₉, R₁₀, and R₁₁ are each independently selected from —H, —Cl, or —OCH₃.

45. The method of claim 42, wherein R₇, R₈, R₉, R₁₀, and R₁₁ are each —H.

46. The method of any one of claims 22-26, wherein X₁ is an optionally substituted heteroarylalkyl.

47. The method of claim 48, wherein X₁ has the structure: 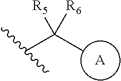
wherein
R₅ and R₆ are each independently selected from —H and alkyl; and
A is an optionally substituted heteroaryl.

48. The method of claim 47, wherein A has the structure:

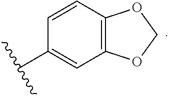 [SMILES: *c1ccc2c(c1)OCO2]

49. The method of claim 1 or 9, wherein the compound of Formula (I) has the structure:

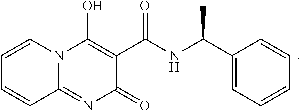 [SMILES: C[C@H](NC(=O)c1c(O)n2ccccc2nc1=O)c1ccccc1]

50. The method of claim 1 or 9, wherein the compound of Formula (I) has the structure:

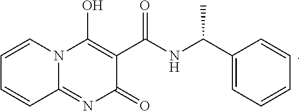 [SMILES: C[C@@H](NC(=O)c1c(O)n2ccccc2nc1=O)c1ccccc1]

51. The method of claim 1 or 9, wherein the compound of Formula (I) is selected from

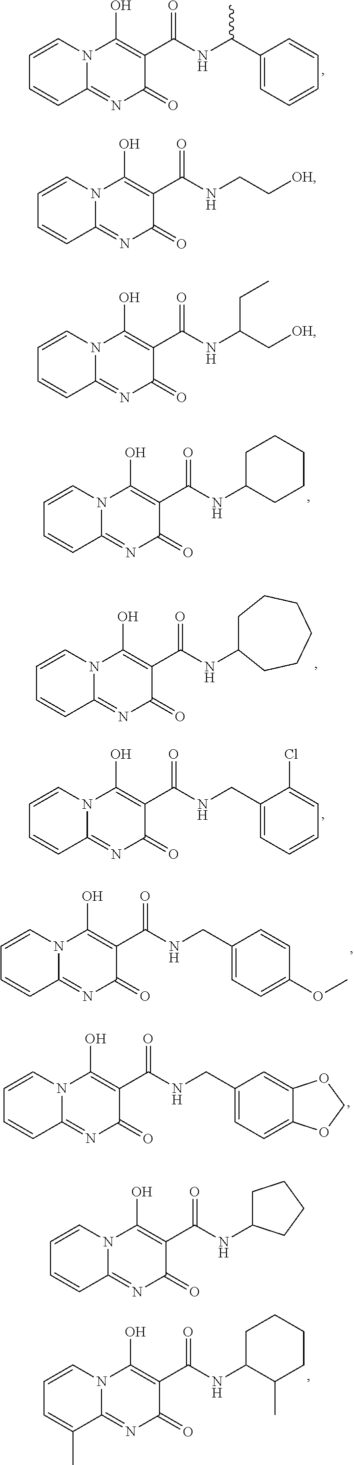

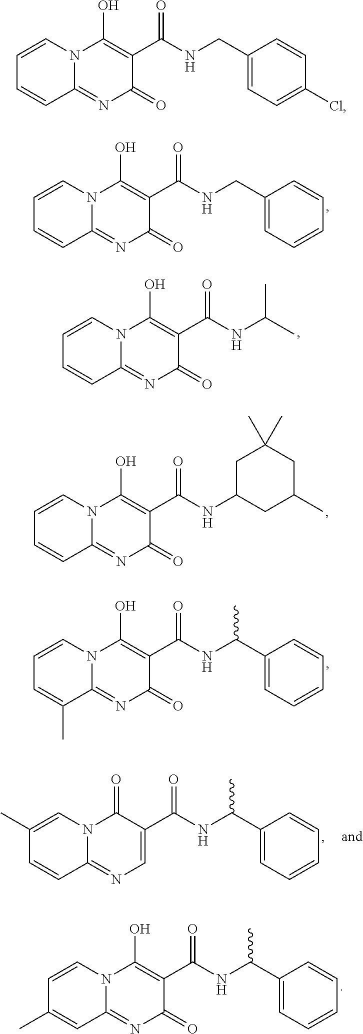

52. The method of any one of claims 1-51, wherein a pharmaceutical composition comprising the compound and a pharmaceutical acceptable excipient is administered to the subject.
